(19) United States
(12) Patent Application Publication
(10) Pub. No.: US 2026/0308620 A1
(43) Pub. Date: Oct. 8, 2026

(54) COMPOSITIONS AND METHODS FOR RECOMBINANT AAV PRODUCTION

(71) Applicant: REGENXBIO Inc., Rockville, MD (US)

(72) Inventors: Jie LI, Rockville, MD (US); Ping LIU, Rockville, MD (US)

(21) Appl. No.: 19/481,341

(22) PCT Filed: May 7, 2024
(86) PCT No.: PCT/US2024/028118

§ 371 Date: Nov. 4, 2025

Related U.S. Application Data
(60) Provisional application No. 63/464,619, filed on May 7, 2023.

(51) Int. Cl. C12N 15/86; C07K 14/005; C07K 14/47; C12N 15/113

(52) U.S. Cl. CPC C12N 15/86; C07K 14/005; C07K 14/4716; C12N 15/113; C12N 2310/531; C12N 2750/14122; C12N 2750/14143; C12N 2750/14152

(57) ABSTRACT

Provided herein are host cells suitable for use in the production of recombinant AAV particles and polypeptides. Also provided herein are methods for producing rAAV particles and polypeptides.

### CROSS-REFRENCE TO RELATED APPLICATIONS

This application claims the benefit of U.S. application No. 63/464,619, filed May 7, 2023, which is incorporated herein by reference in its entirety.

### TECHNICAL FIELD

The present disclosure relates to host cells and their use in a method of producing recombinant adeno-associated virus (rAAV) particles and polypeptides.

### BACKGROUND

Recombinant adeno-associated virus (AAV)-based vectors are currently the most widely used and safest gene therapy products in development. Naso et al., BioDrugs 31:317-334 (2017). The preferred use of rAAV vector systems is due, in part, to the lack of disease associated with the wild-type virus, the ability of AAV to transduce non-dividing as well as dividing cells, and the resulting long-term robust transgene expression observed in clinical trials and that indicate great potential for delivery in gene therapy indications. Additionally, different naturally occurring and recombinant rAAV vector serotypes specifically target different tissues, organs, and cells, and help evade any pre-existing immunity to the vector, thus expanding the therapeutic applications of AAV-based gene therapies. However, the AAV yield limitation with relative low process efficiency as well as high dosing demand on clinical trial restricts AAV's broader application, such as in common diseases including cancers. Before gene therapies based on a replication defective virus, for example, AAV can be more widely adopted for late clinical stage and commercial use, new methods for large scale production of recombinant virus particles need to be developed.

HEK293 cells have been the most widely used cells for recombinant AAV production. The most common platform involves transfecting HEK293 with three plasmids. One plasmid, often referred to as the trans plasmid, carries Rep and Cap genes and encodes proteins for virus replication and capsid formation. A second plasmid, often referred to as the helper plasmid, encodes the essential adenovirus helper genes (E4, E2A, and viral associated (VA) RNAs). And a third plasmid, often referred to as the cis plasmid, contains an expression cassette encoding a gene of interest flanked by two inverted terminal repeats (ITRs), which is incorporated into the rAAV as its genome. In other platforms, HEK293 cells are transfected with two plasmids: one encoding the Rep and Cap genes and helper functions and another encoding the expression cassette flanked by two ITRs.

The prevalent use of HEK293 cells for recombinant AAV production is attributed, in part, to the following advantages: HEK293 cells can be grown in suspension cultures in serum-free media, they are human-originated, and the helper genes E1A and E1B required for AAV production are expressed endogenously by HEK293 cells. Laura Abaandou et al., Cells. 10(7):1667 (2021).

Many factors impact recombinant AAV production, including host cell density, culture medium, whether suspension or adherent cells are used, whether shake flask or bioreactor is used, the harvest time, total amount of DNA used in transfection, and optimal ratios of the three plasmids. See. e.g., Grieger et al., Mol Ther. 24(2): 287-297 (2016); Zhao et al., Mol Ther Methods Clin Dev. 18:312-320 (2020); Joiner et al., Current Opinion in Chemical Engineering 36(11):100823 (2022). Decrease in viability of post-transfected host cells, e.g., HEK293 cells, has been identified as one of the important factors limiting recombinant AAV yield. Several factors contribute to the drop in viability of post-transfected HEK293 cells: the occurrence of apoptosis after plasmid DNA uptake (Li et al, Exp Cell Res. 253(2):541-50 (1999)); ER stress and immune defense induced by the viruses (Li et al., Crit Rev Microbiol. 41(2):150-64 (2015)), and mitochondrial dysfunction and oxidative damage induced by reactive oxygen species (ROS) at high cell density condition (Koo et al, Cell Metab. 28(2):196-206 (2018)).

The effect of genetic modification of host cells on recombinant AAV and polypeptide production remains unknown. Thus, there is a need in the art to improve the productivity and yield of methods for the large-scale production of rAAV particles and recombinant polypeptides by providing improved host cells.

### BRIEF SUMMARY

In one aspect, the disclosure provides a recombinant cell suitable for producing a recombinant virus or polypeptide, wherein the cell comprises at least one modification that reduces or eliminates the activity of at least one endogenous gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway is selected from the group consisting of the ARHGEF7, ARHGEF17, BNIP1, C19orf2, CHST11, DYNLL1, IGFBP3, MAX, and VEGFB gene or gene product. In some embodiments, the at least one endogenous gene or gene product in the apoptosis signaling pathway comprises the DYNLL1 and/or MAX gene or gene product. In some embodiments, the recombinant virus is a recombinant adeno associated virus (AAV). In some embodiments, the recombinant polypeptide is an antibody. In some embodiments, the cell is a HEK293 cell, HEK293 derived cell, CHO cell, CHO derived cell, HeLa cell, SF-9 cell, BHK cell, Vero cell, CAP cell or PerC6 cell. In some embodiments, the cell is a HEK293 cell. In some embodiments, the cell is a HEK293 cell derived cell. In some embodiments, the cell is a recombinant HEK293 cell capable of producing rAAV, wherein the cell comprises at least one modification that reduces or eliminates the activity of at least one endogenous gene or gene selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the cell is a recombinant HEK293 cell capable of producing rAAV, wherein the cell comprises at least one modification that reduces or eliminates the activity of at least one endogenous gene or gene selected from the group consisting of the ARHGEF7, ARHGEF17, BNIP1, C19orf2, CHST11, DYNLL1, IGFBP3, MAX, and VEGFB gene or gene product. In some embodiments, the cell is a recombinant HEK293 cell capable of producing rAAV, wherein the cell comprises at least one modification that reduces or eliminates the activity of at least one endogenous gene or gene selected from the group consisting of the DYNLL1 and MAX gene or gene product. In some embodiments, the cell is a recombinant HEK293 cell capable of producing a recombinant polypeptide (e.g., antibody), wherein the cell comprises at least one modification that reduces or eliminates the activity of at least one endogenous gene or gene selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the cell is a recombinant HEK293 cell capable of producing a recombinant polypeptide (e.g., antibody), wherein the cell comprises at least one modification that reduces or eliminates the activity of at least one endogenous gene or gene selected from the group consisting of the ARHGEF7, ARHGEF17, BNIP1, C19orf2, CHST11, DYNLL1, IGFBP3, MAX, and VEGFB gene or gene product. In some embodiments, the cell is a suspension culture adapted HEK293 cell.

In one aspect, the disclosure provides a recombinant cell capable of producing recombinant virus (e.g., AAV) or polypeptide (e.g., antibody), wherein the cell comprises at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cell is a HEK293 cell. In some embodiments, the cell is a HEK293 cell derived cell.

In one aspect, the disclosure provides a cell bank comprising a plurality of cells, wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1 DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In one aspect, the disclosure provides a cell culture comprising a plurality of cells capable of producing recombinant virus (e.g., AAV) or polypeptide (e.g., antibody), wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells. In some embodiments, the cells are HEK293 cells. In some embodiments, the cells are HEK293 derived cells.

In one aspect, the disclosure provides a method of producing recombinant virus (e.g., AAV) or polypeptide (e.g., antibody), comprising a) providing a cell culture comprising a plurality of cells capable of producing recombinant virus (e.g., AAV) or polypeptide (e.g., antibody); and b) maintaining the cell culture under conditions that allow production of the recombinant virus (e.g., AAV) or polypeptide (e.g., antibody), wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1. ARHGEF7, ARHGEF17, BID, BNIP1, C19orf2, CHST11, CUL1 DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In one aspect, the disclosure provides a method for producing recombinant virus (e.g., AAV) or polypeptide (e.g., antibody), comprising culturing a cell capable of producing recombinant virus (e.g., AAV) or polypeptide (e.g., antibody) under conditions that allow the production of the recombinant virus (e.g., AAV) or polypeptide (e.g., antibody), wherein the cell comprises at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cell is a HEK293 cell. In some embodiments, the cell is a HEK293 cell derived cell.

In one aspect, the disclosure provides a method of increasing the production of recombinant virus (e.g., AAV) or polypeptide (e.g., antibody), comprising a) providing a cell culture comprising a plurality of cells capable of producing recombinant virus (e.g., AAV) or polypeptide (e.g., antibody); and b) maintaining the cell culture under conditions that allow production of the recombinant virus (e.g., AAV) or polypeptide (e.g., antibody), wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1 DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In one aspect, the disclosure provides a method of increasing the production of recombinant virus (e.g., AAV) or polypeptide (e.g., antibody), comprising culturing a cell capable of producing recombinant virus (e.g., AAV) or polypeptide (e.g., antibody) under conditions that allow the production of the recombinant virus (e.g., AAV) or polypeptide (e.g., antibody), wherein the cell comprises at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cell is a HEK293 cell. In some embodiments, the cell is a HEK293 cell derived cell.

In one aspect, the disclosure provides a method of producing rAAV particles, comprising a) providing a cell culture comprising a plurality of cells; b) introducing into the cells one or more polynucleotides encoding at least one of (i) an rAAV genome to be packaged, (ii) adenovirus helper functions necessary for packaging, (iii) an AAV rep protein sufficient for packaging, and (iv) an AAV cap protein sufficient for packaging; and c) maintaining the cell culture under conditions that allow production of the rAAV particles, wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In one aspect, the disclosure provides a method of increasing the production of rAAV particles, comprising a) providing a cell culture comprising a plurality of cells; b) introducing into the cells one or more polynucleotides encoding at least one of (i) an rAAV genome to be packaged, (ii) adenovirus helper functions necessary for packaging, (iii) an AAV rep protein sufficient for packaging, and (iv) an AAV cap protein sufficient for packaging; and c) maintaining the cell culture under conditions that allow production of the rAAV particles, wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In one aspect, the disclosure provides a method of producing a recombinant polypeptide (e.g., antibody), comprising a) providing a cell culture comprising a plurality of cells; b) introducing into the cells one or more polynucleotides encoding the recombinant polypeptide (e.g., antibody); and c) maintaining the cell culture under conditions that allow production of the recombinant polypeptide (e.g., antibody), wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In one aspect, the disclosure provides a method of increasing the production of a recombinant polypeptide (e.g., antibody), comprising a) providing a cell culture comprising a plurality of cells: b) introducing into the cells one or more polynucleotides encoding the recombinant polypeptide (e.g., antibody); and c) maintaining the cell culture under conditions that allow production of the recombinant polypeptide (e.g., antibody), wherein the cells comprise at least one modification that reduces or eliminates the activity of at least one gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway comprises the DYNLL1 and/or MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cells are HEK293 cells. In some embodiments, the cells are HEK293 derived cells.

In some embodiments, the disclosure provides:
[1.] A recombinant cell capable of producing rAAV, wherein the cell comprises at least one modification that reduces or eliminates the activity of at least one endogenous gene or gene product in the apoptosis signaling pathway.
[2.] The cell of [1], wherein the cell comprises at least two modifications that reduce or eliminate the activity of at least two genes or gene products in the apoptosis signaling pathway.
[3.] A cell bank comprising a plurality of cells, wherein the cells comprise at least one modification that reduces or eliminates the activity of at least one endogenous gene or gene product in the apoptosis signaling pathway.
[4.] The cell bank of [3], wherein the cell comprises at least two modifications that reduce or eliminate the activity of at least two genes or gene products in the apoptosis signaling pathway.
[5.] A cell culture comprising a plurality of cells capable of producing rAAV, wherein the cells comprise at least one modification that reduces or eliminates the activity of at least one endogenous gene or gene product in the apoptosis signaling pathway.
[6.] The cell culture of [5], wherein the cells comprise at least two modifications that reduce or eliminate the activity of at least two genes or gene products in the apoptosis signaling pathway.
[7.] A method of producing rAAV particles, comprising
   a) providing a cell culture comprising a plurality of cells capable of producing rAAV; and
   b) maintaining the cell culture under conditions that allow production of the rAAV particles,
   wherein the cells comprise at least one modification that reduces or eliminates the activity of at least one endogenous gene or gene product in the apoptosis signaling pathway.
[8.] A method for producing rAAV particles, comprising culturing a cell capable of producing rAAV particles under conditions that allow the production of the rAAV particles, wherein the cell comprises at least one modification that reduces or eliminates the activity of at least one endogenous gene or gene product in the apoptosis signaling pathway.
[9.] A method of increasing the production of rAAV particles, comprising
   a) providing a cell culture comprising a plurality of cells capable of producing rAAV; and
   b) maintaining the cell culture under conditions that allow production of the rAAV particles,
   wherein the cell comprises at least one modification that reduces or eliminates the activity of at least one endogenous gene or gene product in the apoptosis signaling pathway.
[10.] A method of increasing the production of rAAV particles, comprising culturing a cell capable of producing rAAV particles under conditions that allow the production of the rAAV particles, wherein the cell comprises at least one modification that reduces or eliminates the activity of at least one endogenous gene or gene product in the apoptosis signaling pathway.
[11.] The method of any one of [7] to [10], wherein the cell or cells comprises at least two modifications that reduce or eliminate the activity of at least two genes or gene products in the apoptosis signaling pathway.
[12.] The method of any one of [1] to [11], wherein the cell or cells capable of producing rAAV have been transfected with one or more polynucleotides encoding at least one of
   a) an rAAV genome to be packaged,
   b) adenovirus helper functions necessary for packaging,
   c) an AAV rep protein sufficient for packaging, and
   d) an AAV cap protein sufficient for packaging.
[13.] The method of any one of [1] to [12], wherein the cell or cells capable of producing rAAV have been transfected with one or more polynucleotides encoding
   a) an rAAV genome to be packaged,
   b) adenovirus helper functions necessary for packaging,
   c) an AAV rep protein sufficient for packaging, and
   d) an AAV cap protein sufficient for packaging.
[14.] A method of producing rAAV particles, comprising
   a) providing a cell culture comprising a plurality of cells;
   b) introducing into the cells one or more polynucleotides encoding at least one of
   i. an rAAV genome to be packaged,
   ii. adenovirus helper functions necessary for packaging.
   iii. an AAV rep protein sufficient for packaging, and
   iv. an AAV cap protein sufficient for packaging; and
   c) maintaining the cell culture under conditions that allow production of the rAAV particles,
   wherein the cells comprise at least one modification that reduces or eliminates the activity of at least one endogenous gene or gene product in the apoptosis signaling pathway.
[15.] A method of increasing the production of rAAV particles, comprising
   a) providing a cell culture comprising a plurality of cells;
   b) introducing into the cells one or more polynucleotides encoding at least one of
   i. an rAAV genome to be packaged,
   ii. adenovirus helper functions necessary for packaging,
   iii. an AAV rep protein sufficient for packaging, and
   iv. an AAV cap protein sufficient for packaging; and
   c) maintaining the cell culture under conditions that allow production of the rAAV particles
   wherein the cells comprise at least one modification that reduces or eliminates the activity of at least one endogenous gene or gene product in the apoptosis signaling pathway.
[16.] The method of [14] or [15], wherein the cells comprise at least two modifications that reduce or eliminate the activity of at least two genes or gene products in the apoptosis signaling pathway.
[17.] The method of any one of [14] to [16], comprising introducing into the cells one or more polynucleotides encoding
   i. an rAAV genome to be packaged,
   ii. adenovirus helper functions necessary for packaging.
   iii. an AAV rep protein sufficient for packaging, and
   iv. an AAV cap protein sufficient for packaging.
[18.] The method of any one of [14] to [17], wherein the introducing one or more polynucleotides into the cells is by transfection.
[19.] The cell, cell bank, cell culture or method of any one of [1] to [18], wherein the modification comprises a mutation in the gene.
[20.] The cell, cell bank, cell culture or method of [19], wherein the genetic modification comprises a missense mutation, nonsense mutation, or frameshift mutation.
[21.] The cell, cell bank, cell culture or method of [19], wherein the genetic modification comprises a deletion.
[22.] The cell, cell bank, cell culture or method of any one of [19] to [21], wherein the mutation is a heterozygous mutation.
[23.] The cell, cell bank, cell culture or method of [22], wherein the heterozygous mutation affects one copy of the gene.
[24.] The cell, cell bank, cell culture or method of [22], wherein the heterozygous mutation affects more than on copy of the gene.
[25.] The cell, cell bank, cell culture or method of [22], wherein the heterozygous mutation affects 1, 2 or 3 copies of the gene.
[26.] The cell, cell bank, cell culture or method of any one of [19] to [21], wherein the mutation is a homozygous mutation.
[27.] The cell, cell bank, cell culture or method of any one of [1] to [22], wherein the modification comprises an inhibitory nucleic acid molecule capable of reducing or eliminating the activity of the gene or gene product.
[28.] The cell, cell bank, cell culture or method of [27], wherein the modification comprises an anti-sense RNA.
[29.] The cell, cell bank, cell culture or method of [27], wherein the modification comprises a small interfering RNA (siRNA), microRNA (miRNA), or short hairpin RNA (shRNA).
[30.] The cell, cell bank, cell culture or method of any one of [1] to [29], wherein the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product.
[31.] The cell, cell bank, cell culture or method of [30], wherein the at least one endogenous gene or gene product comprises any two genes or gene products selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product.
[32.] The cell, cell bank, cell culture or method of any one of [1] to [29], wherein the at least one endogenous gene or gene product is selected from the group consisting of the ARHGEF7, ARHGEF17, BNIP1, C19orf2, CHST11, DYNLL1, IGFBP3, MAX, and VEGFB gene or gene product.
[33.] The cell, cell bank, cell culture or method of [32], wherein the at least one endogenous gene or gene product comprises any two genes or gene products selected from the group consisting of the ARHGEF7, ARHGEF17, BNIP1, C19orf2, CHST11, DYNLL1. IGFBP3, MAX, and VEGFB gene or gene product.
[34.] The cell, cell bank, cell culture or method of any one of [1] to [29], wherein the at least one endogenous gene or gene product comprises DYNLL1, IGFBP3, and/or MAX gene or gene product.
[35.] The cell, cell bank, cell culture or method of [34], wherein the at least one endogenous gene or gene product comprises DYNLL1 gene or gene product.
[36.] The cell, cell bank, cell culture or method of [35], wherein the modification comprises a heterozygous DYNLL1 mutation.
[37.] The cell, cell bank, cell culture or method of [34], wherein the at least one endogenous gene or gene product comprises DYNLL1 and MAX gene or gene product.
[38.] The cell, cell bank, cell culture or method of [37], wherein the modification comprises a heterozygous DYNLL1 mutation and a MAX mutation.
[39.] The cell, cell bank, cell culture or method of any one of [1] to [29], wherein the at least one endogenous gene or gene product comprises DYNLL1 and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1. TNFRSF10C, TNFSF12, MAL, MAX, and VEGFB.
[40.] The cell, cell bank, cell culture or method of any one of [1] to [29], wherein the at least one endogenous gene or gene product comprises DYNLL1 and a second gene or gene product selected from the group consisting of ARHGEF7, ARHGEF17, BNIP1, C19orf2, CHST11, IGFBP3, MAX, and VEGFB.
[41.] The cell, cell bank, cell culture or method of any one of [1] to [29], wherein the at least one endogenous gene or gene product comprises DYNLL1 and ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB.
[42.] The cell, cell bank, cell culture or method of any one of [1] to [29], wherein the at least one endogenous gene or gene product comprises DYNLL1 and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB.
[43.] The cell, cell bank, cell culture or method of any one of [1] to [29], wherein the at least one endogenous gene or gene product comprises DYNLL1 and one or more genes or gene products selected from the group consisting of ARHGEF7, ARHGEF17, BNIP1, C19orf2, CHST11, IGFBP3, MAX, and VEGFB.
[44.] The cell, cell bank, cell culture or method of any one of [1] to [29], wherein the at least one endogenous gene or gene product comprises DYNLL1 and ARHGEF7.
[45.] The cell, cell bank, cell culture or method of any one of [1] to [29], wherein the at least one endogenous gene or gene product comprises DYNLL1 and ARHGEF17.
[46.] The cell, cell bank, cell culture or method of any one of [1] to [29], wherein the at least one endogenous gene or gene product comprises DYNLL1 and BNIP1.
[47.] The cell, cell bank, cell culture or method of any one of [1] to [29], wherein the at least one endogenous gene or gene product comprises DYNLL1 and C19orf2.
[48.] The cell, cell bank, cell culture or method of any one of [1] to [29], wherein the at least one endogenous gene or gene product comprises DYNLL1 and CHST11.
[49.] The cell, cell bank, cell culture or method of any one of [1] to [29], wherein the at least one endogenous gene or gene product comprises DYNLL1 and IGFBP3.
[50.] The cell, cell bank, cell culture or method of any one of [1] to [29], wherein the at least one endogenous gene or gene product comprises DYNLL1 and MAX.
[51.] The cell, cell bank, cell culture or method of any one of [1] to [29], wherein the at least one endogenous gene or gene product comprises DYNLL1 and VEGFB.
[52.] The cell, cell bank, cell culture or method of any one of [1] to [51], wherein the cell is a mammalian cell.
[53.] The cell, cell bank, cell culture or method of any one of [1] to [51], wherein the cell is an insect cell.
[54.] The cell, cell bank, cell culture or method of any one of [1] to [51], wherein the cell is a HEK293 cell, HEK293 derived cell, CHO cell, CHO derived cell, HeLa cell, SF-9 cell, BHK cell, Vero cell, CAP cell or PerC6 cell.
[55.] The cell, cell bank, cell culture or method of any one of [1] to [51], wherein the cell is a HEK293 cell.
[56.] The cell, cell bank, cell culture or method of any one of [1] to [55], wherein the cell culture is a suspension culture.
[57.] The cell, cell bank, cell culture or method of any one of [12] to [51], wherein the adenovirus helper functions comprise at least one of an adenovirus E4 gene, E2a gene, and VA gene.
[58.] The cell, cell bank, cell culture or method of any one of [12] to [51], wherein the adenovirus helper functions comprise an adenovirus E4 gene, E2a gene, and VA gene.
[59.] The cell, cell bank, cell culture or method of [58], wherein the polynucleotide encoding the adenovirus helper functions comprises pAD Delta F6 or Helper #5.
[60.] The cell, cell bank, cell culture or method of [58,] wherein the polynucleotide encoding the adenovirus helper functions comprises pHRC #7 or pHRC #8.
[61.] The method of any one of [7] to [60], wherein the maintaining the cell culture or culturing the cell under conditions that allow production of the rAAV particles is for between about 2 days and about 10 days, between about 2 days and about 15 days, or between about 5 days and 14 days.
[62.] The method of any one of [7] to [60], wherein the maintaining the cell culture or culturing the cell under conditions that allow production of the rAAV particles is for about 2 days, about 3 days, about 4 days, about 5 days, about 6 days, or about 7 days.
[63.] The method of any one of [7] to [60], wherein the maintaining the cell culture or culturing the cell under conditions that allow production of the rAAV particles is for about 2 days, about 3 days, about 4 days, about 5 days, about 6 days, or about 7 days.
[64.] The method of [63], wherein the maintaining the cell culture or culturing the cell under conditions that allow production of the rAAV particles is for about 5 days.
[65.] The method of any one of [7] to [64], further comprising recovering the rAAV particles.
[66.] The method of any one of [7] to [65], wherein the method produces more rAAV particles measured as GC/ml than a reference method using a cell that does not comprise the modification.
[67.] The method of any one of [7] to [65], wherein the cell culture produces at least 10%, at least 20%, at least 30%, at least 50%, at least 75%, or at least 100% more rAAV particles measured as GC/ml than a reference method using a cell that does not comprise the modification.
[68.] The cell culture or method of any one of [5] to [67], wherein the cell culture has a volume between about 50 liters and about 20,000 liters.
[69.] The cell culture or method of any one of [5] to [67], wherein the cell culture has a volume between about 200 liters and about 2,000 liters.
[70.] The cell, cell bank, cell culture or method of any one of [1] to [69], wherein the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 serotype.
[71.] The cell, cell bank, cell culture or method of any one of [1] to [69], wherein the rAAV comprises a capsid protein of the AAV8, AAV9, AAV.rh10, AAV.rh20, AAV.rh39, AAV.Rh74, AAV.RHM4-1, AAV.hu32, and AAV.hu37 serotype.
[72.] The cell, cell bank, cell culture or method of any one of [1] to [69], wherein the rAAV s comprises a capsid protein of the AAV8 or AAV9 serotype.
[73.] The cell, cell bank, cell culture or method of any one of [1] to [72], wherein the rAAV comprises a genome encoding a polypeptide or a double stranded RNA molecule.
[74.] The cell, cell bank, cell culture or method of [73], wherein the genome encodes a polypeptide.
[75.] The cell, cell bank, cell culture or method of [73], wherein the genome encodes an anti-VEGF Fab, anti-kallikrein antibody, anti-TNF antibody, microdystrophin, minidystrophin, iduronidase (IDUA), iduronate 2-sulfatase (IDS), low-density lipoprotein receptor (LDLR), tripeptidyl peptidase 1 (TPP1), or non-membrane associated splice variant of VEGF receptor 1 (sFlt-1).
[76.] The cell, cell bank, cell culture or method of [73], wherein the genome encodes an gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-anino acid decarboxylase (AADC), lysosome-associated membrane protein 2 isoform B (LAMP2B), Factor VIII. Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), omithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.
[77.] The cell, cell bank, cell culture or method of [73], wherein the genome encodes a dystrophin or a microdystrophin.
[78.] The cell, cell bank, cell culture or method of [73], wherein the genome to be packaged encodes a microRNA.

Still other features and advantages of the compositions and methods described herein will become more apparent from the following detailed description when read in conjunction with the accompanying drawings.

### BRIEF DESCRIPTION OF THE DRAWINGS

FIG. 1. Functional associations of candidate genes obtained from primary screen.

FIG. 2. Secondary titer verification following siRNA transfection to reduce target gene activity. Helper #5 with appropriate cis and trans plasmids were used to determine titer of AAV8 TG-A, AAV8 TG-B or AAV9 TG-D particles produced by HEK293 cells in 24-DWPs following siRNA transfection.

FIG. 3. AAV titers following siRNA transfection to reduce the activity of 2 target genes. Helper #5 with appropriate cis and trans plasmids were used to determine titer of AAV9 TG-D, AAV8 TG-A and AAV8 TG-B particles produced by HEK293 cells in 24-well plates following siRNA transfection. Relative titer is represented by gray scale.

FIG. 4. Host cell engineering through knocking-out IGFBP3. A) Workflow for single cell cloning of IGFBP3-KO cells. B) AAV titer produced by IGFBP3-KO candidate clones following transfection with the helper #5/TG-A/AAV8 or helper #5/TG-D/AAV9 helper/cis/trans plasmid combinations.

FIG. 5. Host cell engineering through knocking-out IGFBP3. A) Titer Validation of IGFBP3 protein levels in higher AAV producing clones. Results from anti-Vinculin and anti-IGFBP3 Western blots are shown. Ctrl is control cell sample. B-C) AAV titer produced by select IGFBP3-KO candidate clones following transfection with (B) the helper #5/TG-A/AAV8 or (C) helper #5/TG-D/AAV9 helper/cis/trans plasmid combinations.

FIG. 6. Host cell engineering through knocking-out DYNLL1. A) Western blot validation of protein expression level by parental cells after CRISPER-Cas9 DYNLL1-sgRNA-C1/C2 treatment. B) AAV titer produced by DYNLL1-KO candidate clones. AAV titers following transfection with the helper #5/TG-A/AAV8 helper/cis/trans plasmid combination is shown. C) AAV titer produced by select IGFBP3-KO candidate clones following transfection with the helper #5/TG-D/AAV9 helper/cis/trans plasmid combinations. Wt—wild type control parental cells.

FIG. 7. Host cell engineering through knocking-out DYNLL1. Titer validation of selected clones following transfection with A) the pHRC #7 (helper+AAV8-trans) plasmid and TG-A cis plasmid, B) the helper #5, TG-D cis and AAV9 trans plasmids, or C) pHRC #7 (helper+AAV8-trans) plasmid and TG-B cis plasmid. In each panel, from left to right, the clones tested are: Control, 1C1, 4D9. 2C6, 4A8, 3B7, 4D5, and 4A2.

FIG. 8. Host cell engineering through knocking-out DYNLL1 and MAX. AAV titer produced by DYNLL1/MAX KO candidate clones. Titers obtained following transfection with the pHRC #7 (helper+AAV8-trans) plasmid and TG-A cis plasmid is shown.

FIG. 9. Host cell engineering through knocking-out DYNLL1 and MAX. Titer produced by selected clones following transfection with A) the pHRC #7 (helper+AAV8-trans) plasmid and TG-A cis plasmid, B) the pHRC #8 (helper+AAV9-trans) plasmid and TG-C cis plasmid. C) the helper #5, TG-D cis and AAV9 trans plasmids, and D) the pHRC #7 (helper+AAV8-trans) plasmid and TG-B cis plasmid are shown. In each panel, from left to right, the clones tested are: HEK293 parent control, 4A2 (DYNLL1+/−) control, 4A2-3D3, 4A2-3B11, 4A2-3E6, 4A2-1H7, 4A2-4F12, 4A2-2C12, 4A2-3C3, and 4A2-2H7.

### DETAILED DESCRIPTION

In one aspect, the disclosure provides a recombinant cell suitable for producing a recombinant virus (e.g., AAV) or polypeptide (e.g., antibody), wherein the cell comprises at least one modification that reduces or eliminates the activity of at least one endogenous gene or gene product in the apoptosis signaling pathway. In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway is selected from the group consisting of the ARHGEF7, ARHGEF17, BNIP1, C19orf2, CHST11, DYNLL1, IGFBP3. MAX, and VEGFB gene or gene product. In some embodiments, the at least one endogenous gene or gene product in the apoptosis signaling pathway is selected from the group consisting of the DYNLL1 and MAX gene or gene product. In some embodiments, the at least one endogenous gene or gene product in the apoptosis signaling pathway comprises the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product in the apoptosis signaling pathway comprises the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the cell is a HEK293 cell, HEK293 derived cell, CHO cell, CHO derived cell, HeLa cell, SF-9 cell, BHK cell, Vero cell, CAP cell or PerC6 cell. In some embodiments, the cell is a HEK293 cell. In some embodiments, the cell is a HEK293 cell derived cell. In some embodiments, the cell is a recombinant HEK293 cell capable of producing rAAV. In some embodiments, the cell is a recombinant HEK293 cell capable of producing a recombinant polypeptide (e.g., antibody). In some embodiments, the cell is a suspension culture adapted HEK293 cell.

In further aspects, provided herein are cell banks comprising a plurality of cells described herein and cell cultures comprising a plurality of cells described herein. Also provided are methods of producing a recombinant virus (e.g., AAV) or polypeptide (e.g., antibody) using cells described herein and methods of increasing the production of a recombinant virus (e.g., AAV) or polypeptide (e.g., antibody) using cells described herein.

### Definitions

Unless defined otherwise, all technical and scientific terms used herein have the same meaning as commonly understood by one of ordinary skill in the art to which this disclosure is related. To facilitate an understanding of the disclosed methods, a number of terms and phrases are defined below.

The term “HEK293 cell” refers to any cell whose lineage can ultimately be traced back to the original HEK293 cell line represented by ATCC catalog number CRL-1573™ and/or generated upon transforming human embryonic kidney cells with fragments of adenovirus type 5 DNA as described (Graham et al. (1977) J. Gen. Virol. 36:59-74). In some embodiments, the HEK293 cell comprises a modification that reduces or eliminates the activity of at least one gene or gene product selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the HEK293 cell comprises a mutation that reduces or eliminates the activity of the DYNLL1 gene or gene product. In some embodiments, the HEK293 cell comprises a mutation that reduces or eliminates the activity of the IGFBP3 gene or gene product. In some embodiments, the HEK293 cell comprises a mutation that reduces or eliminates the activity of the MAX gene or gene product. In some embodiments, the HEK293 cell comprises mutations that reduce or eliminate the activity of the DYNLL1 and MAX gene or gene product. In some embodiments, the HEK293 cell comprises a mutation that reduces or eliminates the activity of the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the HEK293 cell comprises a mutation that reduces or eliminates the activity of the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the HEK293 cell is a HEK293 derived cell that was derived from a starting population of HEK293 cells by single cell cloning to select a cell with a desirable property, for example, high level of recombinant AAV production or high level of recombinant polypeptide (e.g., antibody) production. In some embodiments, the HEK293 cell is a HEK293 derived cell that was derived from a starting population of HEK293 cells by single cell cloning to select a cell capable of producing high recombinant AAV titer.

In some embodiments, the HEK293 cell is a cell that has been genetically modified, e.g., by introducing at least one modification that reduces or eliminates the activity of at least one gene or gene product selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the HEK293 cell is a cell that has been genetically modified, e.g., by introducing at least one modification that reduces or eliminates the activity of at least one gene or gene product selected from the group consisting of the DYNLL1, IGFBP3, and MAX gene or gene product. In some embodiments, the HEK293 cell is a cell that has been genetically modified to reduce or eliminate the activity of the DYNLL1 and/or MAX gene or gene product. In some embodiments, the HEK293 cell is a cell that has been genetically modified to reduce or eliminate the activity of the DYNLL1 gene or gene product and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the HEK293 cell is a cell that has been genetically modified to reduce or eliminate the activity of the DYNLL1 gene or gene product and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the HEK293 cell is a cell that has been genetically modified (1) by introducing at least one modification that reduces or eliminates the activity of at least one gene or gene product selected from the group consisting of the ANGPT1, ARHGEF7, ARHGEF17, BID, BNIP1, C19orf2, CHST11, CULT, DAP3, DYNLL1, FIS1, HMGB2, IGFBP3, NRG1, TNFRSF10C, TNFSF12, MAL, MAX, and VEGFB gene or gene product and (2) by transfecting with one or more polynucleotides encoding at least one of an rAAV genome to be packaged, adenovirus helper functions necessary for packaging, an AAV rep protein sufficient for packaging, and an AAV cap protein sufficient for packaging. In some embodiments, the HEK293 cell is a cell that has been genetically modified (1) by introducing at least one modification that reduces or eliminates the activity of at least one gene or gene product selected from the group consisting of the ANGPT1, ARHGEF7, ARHGEF17, BID, BNIP1. C19orf2, CHST11, CUL1 DAP3, DYNLL1, FIS1, HMGB2, IGFBP3, NRG1, TNFRSF10C, TNFSF12, MAL, MAX, and VEGFB gene or gene product and (2) by transfecting with a recombinant polynucleotide encoding a recombinant polypeptide of interest (e.g., an antibody).

“AAV” is an abbreviation for adeno-associated virus and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), avian AAV, bovine AAV, canine AAV, equine AAV, primate AAV, non-primate AAV, and ovine AAV, and modifications, derivatives, or pseudotypes thereof. “Primate AAV” refers to AAV that infects primates, “non-primate AAV” refers to AAV that infects non-primate mammals, “bovine AAV” refers to AAV that infects bovine mammals, etc.

“Recombinant”, as applied to an AAV particle means that the AAV particle is the product of one or more procedures that result in an AAV particle construct that is distinct from an AAV particle in nature.

A recombinant adeno-associated virus particle “rAAV particle” refers to a viral particle composed of at least one AAV capsid protein and an encapsidated polynucleotide rAAV vector genome comprising a heterologous polynucleotide (i.e., a polynucleotide other than a wild-type AAV genome such as a transgene to be delivered to a mammalian cell). The rAAV particle may be of any AAV serotype, including any modification, derivative or pseudotype (e.g., AAV1, AAV2, AAV3, AAV4, AAV5, AAV6, AAV7, AAV8, AAV9, or AAV10, or derivatives/modifications/pseudotypes thereof). Such AAV serotypes and derivatives/modifications/pseudotypes, and methods of producing such serotypes/derivatives/modifications/pseudotypes are known in the art (see, e.g., Asokan et al., Mol. Ther. 20(4):699-708 (2012).

The rAAV particles of the disclosure may be of any serotype, or any combination of serotypes, (e.g., a population of rAAV particles that comprises two or more serotypes, e.g., comprising two or more of rAAV2, rAAV8, and rAAV9 particles). In some embodiments, the rAAV particles are rAAV1, rAAV2, rAAV3, rAAV4, rAAV5, rAAV6, rAAV7, rAAV8, rAAV9, rAAV10, or other rAAV particles, or combinations of two or more thereof). In some embodiments, the rAAV particles are rAAV8 or rAAV9 particles.

In some embodiments, the rAAV particles have an AAV capsid protein of a serotype selected from the group consisting of AAV1, AAV2, AAV3, AAV4, AAV5, AAV6, AAV7, AAV8, AAV9, AAV10, AAV11, AAV12, AAV13, AAV14, AAV15 and AAV16 or a derivative, modification, or pseudotype thereof. In some embodiments, the rAAV particles have an AAV capsid protein of a serotype of AAV8, AAV9, or a derivative, modification, or pseudotype thereof.

The term “antibody” means an immunoglobulin molecule (or a group of immunoglobulin molecules) that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the terms “antibody” and “antibodies” are terms of art and can be used interchangeably herein and refer to a molecule with an antigen-binding site that specifically binds an antigen.

Antibodies can include, for example, monoclonal antibodies, recombinantly produced antibodies, human antibodies, humanized antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multi-specific antibodies. Antibodies can be of any type (e.g., IgG, IgE, IgM, IgD, IgA, or IgY), any class (e.g., IgG₁, IgG₂, IgG₃, IgG₄, IgA₁, or IgA₂), or any subclasses (isotypes) thereof (e.g. IgG1, IgG2, IgG3, IgG4, IgA1 and IgA2), of immunoglobulin molecule, based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated or fused to other molecules such as toxins, radioisotopes, other polypeptides etc.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure. In some embodiments, the term “cell culture” refers to cells grown in suspension. In some embodiments, the term “cell culture” refers to adherent cells grown attached to microcarriers or macrocarriers in stirred bioreactors. In some embodiments, the term “cell culture” refers to cells grown in a perfusion culture. In some embodiments, the term “cell culture” refers to cells grown in an alternating tangential flow (ATF) supported high-density perfusion culture.

The terms “purifying”, “purification”, “separate”, “separating”, “separation”, “isolate”, “isolating”, or “isolation”, as used herein, refer to increasing the degree of purity of a target product, e.g., rAAV particles and rAAV genome from a sample comprising the target product and one or more impurities. Typically, the degree of purity of the target product is increased by removing (completely or partially) at least one impurity from the sample. In some embodiments, the degree of purity of the rAAV in a sample is increased by removing (completely or partially) one or more impurities from the sample by using a method described herein.

“About” modifying, for example, the quantity of an ingredient in the compositions, concentration of an ingredient in the compositions, flow rate, rAAV particle yield, feed volume, salt concentration, and like values, and ranges thereof, employed in the methods provided herein, refers to variation in the numerical quantity that can occur, for example, through typical measuring and handling procedures used for making concentrates or use solutions; through inadvertent error in these procedures; through differences in the manufacture, source, or purity of the ingredients employed to make the compositions or carry out the methods; and like considerations. The term “about” also encompasses amounts that differ due to aging of a composition with a particular initial concentration or mixture. The term “about” also encompasses amounts that differ due to mixing or processing a composition with a particular initial concentration or mixture. Whether or not modified by the term “about” the claims include equivalents to the quantities. In some embodiments, the term “about” refers to ranges of approximately 10-20% greater than or less than the indicated number or range. In further embodiments, “about” refers to plus or minus 10% of the indicated number or range. For example, “about 10%” indicates a range of 9% to 1100.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Where embodiments of the disclosure are described in terms of a Markush group or other grouping of alternatives, the disclosed method encompasses not only the entire group listed as a whole, but also each member of the group individually and all possible subgroups of the main group, and also the main group absent one or more of the group members. The disclosed methods also envisage the explicit exclusion of one or more of any of the group members in the disclosed methods.

### Recombinant Cells

In one aspect, the disclosure provides a recombinant cell suitable for producing a recombinant virus (e.g., AAV) or polypeptide (e.g., antibody), wherein the cell comprises at least one modification that reduces or eliminates the activity of at least one endogenous gene or gene product in the apoptosis signaling pathway. In some embodiments, the cell is an immortalized cell. In some embodiments, the cell is a mammalian cell. In some embodiments, the cell is an immortalized mammalian cell. In some embodiments, the cell is a HEK293 cell, HEK293 derived cell, CHO cell, CHO derived cell, HeLa cell, SF-9 cell, BHK cell, Vero cell, CAP cell or PerC6 cell. In some embodiments, the cell is a HEK293 cell. In some embodiments, the cell is a HEK293 derived cell.

In some embodiments, the cell is an adherent cell. In some embodiments, the cell is a suspension cell. In some embodiments, the cell is a suspension adapted HEK293 cell. In some embodiments, the cell is a suspension adapted HEK293 derived cell.

In some embodiments, the at least one endogenous gene or gene product in the apoptosis signaling pathway is selected from the group consisting of the ANGPT1, ARHGEF7, ARHGEF17, BID, BNIP1, C19orf2, CHST11, CUL1 DAP3, DYNLL1, FIS1, HMGB2, IGFBP3, NRG1, TNFRSF10C, TNFSF12, MAL, MAX, and VEGFB gene or gene product. In some embodiments, the at least one endogenous gene or gene product in the apoptosis signaling pathway is selected from the group consisting of the ARHGEF7, ARHGEF17, BNIP1, C19orf2, CHST11, DYNLL1, IGFBP3, MAX, and VEGFB gene or gene product. In some embodiments, the at least one endogenous gene or gene product in the apoptosis signaling pathway is selected from the group consisting of the DYNLL1, IGFBP3, and MAX gene or gene product. In some embodiments, the at least one endogenous gene or gene product comprises the DYNLL1 and/or MAX gene or gene product. In some embodiments, the at least one endogenous gene or gene product comprises the DYNLL1 gene or gene product and a second gene or gene product selected from the group consisting of the ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB. In some embodiments, the at least one endogenous gene or gene product comprises the DYNLL1 gene or gene product and one or more genes or gene products selected from the group consisting of the ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB.

The human angiopoietin 1 gene (also known as ANGPT1, AGP1, AGPT, AGPT-1, ANGl, and HAE5) encodes a secreted glycoprotein that belongs to the angiopoietin family. The angiopoietin 1 protein binds and activates TEK/TIE2 receptor by inducing its dimerization and tyrosine phosphorylation. It plays an important role in the regulation of angiogenesis, endothelial cell survival, proliferation, migration, adhesion and cell spreading, reorganization of the actin cytoskeleton, but also maintenance of vascular quiescence. In quiescent vessels, ANGPT1 oligomers recruit TEK to cell-cell contacts, forming complexes with TEK molecules from adjoining cells, and this leads to preferential activation of phosphatidylinositol 3-kinase and the AKT1 signaling cascades. In migrating endothelial cells that lack cell-cell adhesions, ANGT1 recruits TEK to contacts with the extracellular matrix, leading to the formation of focal adhesion complexes, activation of PTK2/FAK and of the downstream kinases MAPK1/ERK2 and MAPK3/ERK1, and ultimately to the stimulation of sprouting angiogenesis. In some embodiments, the human ANGPT1 gene refers to the gene described by NCBI Gene ID No. 284. In some embodiments, the human ANGPT1 gene encodes the human ANGPT1 mRNA of NCBI Accession No. NM_001146. In some embodiments, the human ANGPT1 gene encodes one or more of the following human ANGPT1 mRNA variants: NCBI Accession No. NM_001146.5, NM_001199859.3, NM 001314051.1, and XR 928319.2. In some embodiments, the human ARHGEF7 gene encodes one or more of the following human ARHGEF7 polypeptide isoforms: UniProt Accession No. Q15389-1, and Q15389-2.

The human Rho guanine nucleotide exchange factor 7 gene (also known as ARHGEF7, P85SPR, COOL1, PIXB, P85, PAK-Interacting Exchange Factor Beta, Nbla10314, KIAA0142, BETA-PIX, P85COOL1, P50BP, PAK3, P50, Rho Guanine Nucleotide Exchange Factor (GEF) 7, SH3 Domain-Containing Proline-Rich Protein, DKFZp686C12170, DKFZp761K1021, COOL-1, Beta-Pix, PAK3BP) encodes a protein that belongs to a family of cytoplasmic proteins that activate the Ras-like family of Rho proteins by exchanging bound GDP for GTP. The ARHGEF7 protein forms a complex with the small GTP binding protein Rac1 and recruits Rac1 to membrane ruffles and to focal adhesions. Multiple alternatively spliced transcript variants encoding different isoforms have been observed for this gene. In some embodiments, the human ARHGEF7 gene refers to the gene described by NCBI Gene ID No. 8874. In some embodiments, the human ARHGEF7 gene encodes the human ARHGEF7 mRNA of NCBI Accession No. NM_003899.5. In some embodiments, the human ARHGEF7 gene encodes one or more of the following human ARHGEF7 mRNA variants: NCBI Accession No. NM_001113511.2, NM_001113512.2, NM_001113513.2, NM_001320851.2, NM_001320852.1. NM_001320853.2, NM_001320854.2, NM_001330597.2, NM_001330598.2, NM_001354046.1, NM_001354047.1, NM_001354048.1, NM_001354049.2, NM_001354050.2, NM_001354051.2, NM_001354052.2, NM_001354053.2, NM_001354054.2, NM_001354055.2, NM_001354056.1, NM_001354057.2, NM_001354058.2, NM_001354059.2, NM_001354060.2, NM_001354061.2, NM_003899.5, NM_145735.3, XM 006719956.3, XM 011521133.2, XM_017020814.1, XM 017020815.1, XM_017020816.1, XM_017020819.1, XM_017020820.1. XM_017020821.2, XM 017020822.1, XM 017020823.2, XM 017020828.1, XM 017020829.2, XM 024449428.1, XR_001749712.1, and XR_002957481.1. In some embodiments, the human ARHGEF7 gene encodes one or more of the following human ARHGEF7 polypeptide isoforms: UniProt Accession No. Q14155-1, Q14155-2, Q14155-3, Q14155-4. Q14155-5, and Q14155-6.

The human Rho guanine nucleotide exchange factor (GEF) 17 gene (also known as ARHGEF17, TEM4, RHOGEF17, P164RHOGEF, p164-RhoGEF) encodes a protein that belongs to a family of cytoplasmic proteins that activate the Ras-like family of Rho proteins by exchanging bound GDP for GTP. The ARHGEF7 protein acts as guanine nucleotide exchange factor (GEF) for RhoA GTPases. Multiple alternatively spliced transcript variants encoding different isoforms have been observed for this gene. In some embodiments, the human ARHGEF17 gene refers to the gene described by NCBI Gene ID No. 9828. In some embodiments, the human ARHGEF17 gene encodes the human ARHGEF17 mRNA of NCBI Accession No. NM_014786. In some embodiments, the human ARHGEF17 gene encodes one or more of the following human ARHGEF17 mRNA variants: NCBI Accession No. NM_014786.4, XM 017018623.1, XM_017018624.1, XR_001748051.1, XR_001748052.1, and XR_950116.2. In some embodiments, the human ARHGEF17 gene encodes the human ARHGEF7 polypeptide of UniProt Accession No. Q96PE2.

The human BH3 interacting domain death agonist gene (also known as BID, and FP497) encodes a death agonist that heterodimerizes with either agonist BAX or antagonist BCL2, and thus regulate apoptosis. BID is a member of the BCL-2 family of cell death regulators. It is a mediator of mitochondrial damage induced by caspase-8 (CASP8). CASP8 cleaves BID, and the COOH-terminal part translocates to mitochondria where it triggers cytochrome c release. Multiple alternatively spliced transcript variants have been found. In some embodiments, the human BID gene refers to the gene described by NCBI Gene ID No. 637. In some embodiments, the human BID gene encodes the human BID mRNA of NCBI Accession No. NM_001196. In some embodiments, the human BID gene encodes one or more of the following human BID mRNA variants: NCBI Accession No. NM_001196.3, NM_001244567.1, NM_001244569.1, NM_001244570.1, NM_001244572.1, NM 197966.2, and NM 197967.2. In some embodiments, the human BID gene encodes one or more of the human BID polypeptide isoforms: UniProt Accession No. P55957-1. P55957-2, P55957-3, and P55957-4.

The human BCL2/adenovirus E1B 19 kDa interacting protein 1 gene (also known as BNIP1, BCL2 Interacting Protein 1, SEC20, TRG-8 and Nip1) is a member of the BCL2/adenovirus E1B 19 kd-interacting protein (BNIP) family. BNIP1 interacts with the E1B 19 kDa protein, which protects cells from virally induced cell death. BNIP1 also interacts with E1B 19 kDa-like sequences of BCL2, another apoptotic protector. In addition, BNIP1 is involved in vesicle transport into the endoplasmic reticulum. In some embodiments, the human BNIP1 gene refers to the gene described by NCBI Gene ID No. 662. In some embodiments, the human BNIP1 gene encodes the human BNIP1 mRNA of NCBI Accession No. NM_001205. In some embodiments, the human BNIP1 gene encodes one or more of the following human BNIP1 mRNA variants: NCBI Accession No. NM_001205.3, NM 013978.3, NM 013979.3, NM 013980.3, XM_011534638.1 and XM_011534639.1. In some embodiments, the human BNIP1 gene encodes one or more of the human BNIP1 polypeptide isoforms: UniProt Accession No. Q12981-1, Q12981-2, Q12981-3 and Q12981-4.

The human chromosome 19 open reading frame 2 gene (also known as C19orf2, URI1, RMP, URI, NNX3 and PPP1R19) is a member of the prefoldin family of molecular chaperones. The C19orf2 protein functions as a scaffolding protein and plays roles in ubiquitination and transcription, in part though interactions with the RNA polymerase II subunit RPB5. C19orf2 may play a role in multiple malignancies including ovarian cancer and hepatocellular carcinoma. In some embodiments, the human C19orf2 gene refers to the gene described by NCBI Gene ID No. 8725. In some embodiments, the human C19orf2 gene encodes the human C19orf2 mRNA of NCBI Accession No. NM_003796. In some embodiments, the human C19orf2 gene encodes one or more of the following human C19orf2 mRNA variants: NCBI Accession No. NM_001252641.2, NM_003796.3, NR_045557.1, XM 005259362.2, XM_005259363.4, XM_011527435.2 and XM_024451751.1. In some embodiments, the human C19orf2 gene encodes one or more of the human C19orf2 polypeptide isoforms: UniProt Accession No. O94763-1, O94763-2. O94763-3 and O94763-4.

The human carbohydrate (chondroitin 4) sulfotransferase 11 gene (also known as CHST11, C4ST, C4ST1, OCBMD, C4ST-1 and HSA269537) encodes a protein that belongs to the sulfotransferase 2 family. CHST11 is localized to the Golgi membrane and catalyzes the transfer of sulfate to position 4 of the N-acetylgalactosamine (GalNAc) residue of chondroitin. Chondroitin sulfate constitutes the predominant proteoglycan present in cartilage and is distributed on the surfaces of many cells and extracellular matrices. A chromosomal translocation involving CHST11 and IgH, t(12; 14)(q23:q32), has been reported in a patient with B-cell chronic lymphocytic leukemia. In some embodiments, the human CHST11 gene refers to the gene described by NCBI Gene ID No. 50515. In some embodiments, the human CHST11 gene encodes the human CHST11 mRNA of NCBI Accession No. NM_018413. In some embodiments, the human CHST11 gene encodes one or more of the following human CHST11 mRNA variants: NCBI Accession No. NM_001173982.2, NM_018413.6 and XM_017019369.1. In some embodiments, the human CHST11 gene encodes one or more of the human CHST11 polypeptide isoforms: UniProt Accession No. Q9NPF2-1 and Q9NPF2-2.

The human cullin 1 gene (also known as CUL1) encodes a protein that is predicted to enable ubiquitin protein ligase binding activity and ubiquitin-protein transferase activity. CUL1 is involved in SCF-dependent proteasomal ubiquitin-dependent protein catabolic process and protein ubiquitination. CUL1 is located in plasma membrane, is part of Parkin-FBXW7-Cull ubiquitin ligase complex and SCF ubiquitin ligase complex. In some embodiments, the human CUL1 gene refers to the gene described by NCBI Gene ID No. 8454. In some embodiments, the human CUL1 gene encodes the human CUL1 mRNA of NCBI Accession No. NM_003592. In some embodiments, the human CUL1 gene encodes one or more of the following human CUL1 mRNA variants: NCBI Accession No. NM_001370660.1, NM_001370661.1, NM 001370662.1, NM_001370663.1, NM_001370664.1 and NM_003592.3. In some embodiments, the human CUL1 gene encodes the human CUL1 polypeptide of UniProt Accession No. Q13616.

The human death associated protein 3 gene (also known as DAP3, DAP-3, S29mt, MRPS29, MRP-S29 and bMRP-10) encodes a mitoribosomal 28S subunit protein that also participates in apoptotic pathways which are initiated by tumor necrosis factor-alpha, Fas ligand and gamma interferon. DAP3 potentially binds ATP/GTP and might be a functional partner of the mitoribosomal protein S27. In some embodiments, the human DAP3 gene refers to the gene described by NCBI Gene ID No. 7818. In some embodiments, the human DAP3 gene encodes the human DAP3 mRNA of NCBI Accession No. NM_004632. In some embodiments, the human DAP3 gene encodes one or more of the following human DAP3 mRNA variants: NCBI Accession No. NM_001199849.1, NM_001199850.1, NM_001199851.1, NM 004632.4, NM 033657.2, XM_005245480.2, XM_005245481.2, XM 017002289.1, XM_017002290.1, XM_017002291.1, XM 017002292.1, XM_017002293.1, XM_017002294.1. XM_017002295.1, XM 024449697.1, XM_024449698.1 and XM_024449700.1. In some embodiments, the human DAP3 gene encodes one or more of the human DAP3 polypeptide isoforms: UniProt Accession No. P51398-1, P51398-2 and P51398-3.

The human dynein, light chain, LC8-type 1 gene (also known as DYNLL1, LC8, PIN, DLC1, DLC8, LC8a, DNCL1, hdlc1 and DNCLC1) encodes a cytoplasmic dynein component. Cytoplasmic dyneins are large enzyme complexes with a molecular mass of about 1,200 kD. They contain two force-producing heads formed primarily from dynein heavy chains and stalks linking the heads to a basal domain, which contains a varying number of accessory intermediate chains. The complex is involved in intracellular transport and motility. DYNLL1 is a light chain and exists as part of this complex but also physically interacts with and inhibits the activity of neuronal nitric oxide synthase. Binding of DYNLL1 destabilizes the neuronal nitric oxide synthase dimer, a conformation necessary for activity and it may regulate numerous biologic processes through its effects on nitric oxide synthase activity. In some embodiments, the human DYNLL1 gene refers to the gene described by NCBI Gene ID No. 8655. In some embodiments, the human DYNLL1 gene encodes the human DYNLL1 mRNA of NCBI Accession No. NM_003746. In some embodiments, the human DYNLL1 gene encodes one or more of the following human DYNLL1 mRNA variants: NCBI Accession No. NM_001037494.2. NM_001037495.2 and NM_003746.3. In some embodiments, the human DYNLL1 gene encodes the human DYNLL1 polypeptide of UniProt Accession No. P63167.

The human fission 1 (mitochondrial outer membrane) homolog (S. cerevisiae) gene (also known as FIS1, TTC11 and CGI-135) encodes a protein involved in several processes, including calcium-mediated signaling using intracellular calcium source; cellular calcium ion homeostasis; and mitochondrion organization. FIS1 acts upstream of or within mitochondrion morphogenesis. FIS1 is located in mitochondrion and peroxisome. FIS1 is an integral component of mitochondrial outer membrane and integral component of peroxisomal membrane. In some embodiments, the human FIS1 gene refers to the gene described by NCBI Gene ID No. 51024. In some embodiments, the human FIS1 gene encodes the human FIS1 mRNA of NCBI Accession No. NM_016068. In some embodiments, the human FIS1 gene encodes the human FIS1 polypeptide of UniProt Accession No. Q9Y3D6.

The human high-mobility group box 2 gene (also known as HMGB2 and HMG2) encodes a member of the non-histone chromosomal high mobility group protein family. The proteins of this family are chromatin-associated and ubiquitously distributed in the nucleus of higher eukaryotic cells. In vitro studies have demonstrated that HMGB2 is able to efficiently bend DNA and form DNA circles. These studies suggest a role in facilitating cooperative interactions between cis-acting proteins by promoting DNA flexibility. HMGB2 was also reported to be involved in the final ligation step in DNA end-joining processes of DNA double-strand breaks repair and V(D)J recombination. In some embodiments, the human HMGB2 gene refers to the gene described by NCBI Gene ID No. 3148. In some embodiments, the human HMGB2 gene encodes the human HMGB2 mRNA of NCBI Accession No. NM_002129. In some embodiments, the human HMGB2 gene encodes one or more of the following human HMGB2 mRNA variants: NCBI Accession No. NM_001130688.1, NM_001130689.1 and NM_002129.4. In some embodiments, the human HMGB2 gene encodes the human HMGB2 polypeptide of UniProt Accession No. P26583.

The human insulin-like growth factor binding protein 3 gene (also known as IGFBP3, IBP3 and BP-53) is a member of the insulin-like growth factor binding protein (IGFBP) family and encodes a protein with an IGFBP domain and a thyroglobulin type-I domain. IGFBP3 forms a temary complex with insulin-like growth factor acid-labile subunit (IGFALS) and either insulin-like growth factor (IGF) I or II. IGFBP3 also exhibits IGF-independent antiproliferative and apoptotic effects mediated by its receptor TMEM219/IGFBP-3R. In some embodiments, the human IGFBP3 gene refers to the gene described by NCBI Gene ID No. 3486. In some embodiments, the human IGFBP3 gene encodes the human IGFBP3 mRNA of NCBI Accession No. NM_001013398. In some embodiments, the human IGFBP3 gene encodes one or more of the following human IGFBP3 mRNA variants: NCBI Accession No. NM_000598.5 and NM_001013398.2. In some embodiments, the human IGFBP3 gene encodes one or more of the human IGFBP3 polypeptide isoforms: UniProt Accession No. P17936-1 and P17936-2.

The human neuregulin 1 gene (also known as NRG1. GGF, HGL, HRG, NDF, ARIA, GGF2, HRG1, HRGA, SMDF, MST131, MSTP131 and NRGT-IT2) encodes a membrane glycoprotein that mediates cell-cell signaling and plays a critical role in the growth and development of multiple organ systems. A variety of different isoforms are produced from this gene through alternative promoter usage and splicing. These isoforms are expressed in a tissue-specific manner and differ significantly in their structure and are classified as types I, II, III. IV. V and VI. Dysregulation of NRG1 has been linked to diseases such as cancer, schizophrenia and bipolar disorder (BPD). NRG1 consists of NH2-terminal ECD (extracellular structural domains), transmembrane structural domains and highly conserved COOH-terminal ICD (intracellular structural domains). Ectopic expression of NRG1 leads to NRG1-ICD dependent apoptosis. In some embodiments, the human NRG1 gene refers to the gene described by NCBI Gene ID No. 3084. In some embodiments, the human NRG1 gene encodes the human NRG1 mRNA of NCBI Accession No. NM_004495. In some embodiments, the human NRG1 gene encodes one or more of the following human NRG1 mRNA variants: NCBI Accession No. NM_001159995.3, NM_001159996.2, NM_001159999.3, NM_001160001.3, NM_001160002.2, NM_001160004.3, NM_001160005.1, NM_001160007.2, NM_001160008.2, NM_001322197.2, NM_001322201.2, NM_001322202.2, NM_001322205.2, NM_001322206.2, NM_001322207.2, NM_004495.4, NM 013956.5, NM_013957.5, NM 013958.3, NM 013959.3, NM_013960.5, NM_013962.2, NM 013964.5, XM_005273486.3, XM_005273487.3, XM 006716335.3, XM_011544512.2, XM 017013365.2, XM 017013366.2, XM 017013367.1, XM 017013368.2, XM 017013369.2, XM 017013370.1, XM 017013371.2, XM_017013372.2 and XM_024447143.1. In some embodiments, the human NRG1 gene encodes one or more of the human NRG1 polypeptide isoforms: UniProt Accession No. Q02297-1, Q02297-2, Q02297-3, Q02297-4, Q02297-5, Q02297-6, Q02297-7. Q02297-8, Q02297-9, Q02297-10 and Q02297-11.

The human tumor necrosis factor receptor superfamily, member 10c, decoy without an intracellular domain gene (also known as TNFRSF10C, LIT, DCR1, TRID, CD263, TRAILR3, TRAIL-R3 and DCR1-TNFR) encodes a member of the TNF-receptor superfamily. TNFRSF10C contains an extracellular TRAIL-binding domain and a transmembrane domain, but no cytoplasmic death domain. TNFRSF10C is not capable of inducing apoptosis and is thought to function as an antagonistic receptor that protects cells from TRAIL-induced apoptosis. TNFRSF10C was found to be a p53-regulated DNA damage-inducible gene. The expression of TNFRSF10C was detected in many normal tissues but not in most cancer cell lines, which may explain the specific sensitivity of cancer cells to the apoptosis-inducing activity of TRAIL. In some embodiments, the human TNFRSF10C gene refers to the gene described by NCBI Gene ID No. 8794. In some embodiments, the human TNFRSF10C gene encodes the human TNFRSF10C mRNA of NCBI Accession No. NM_003841. In some embodiments, the human TNFRSF10C gene encodes the human TNFRSF10C polypeptide of UniProt Accession No. 014798.

The human tumor necrosis factor (ligand) superfamily, member 12 gene (also known as TNFSF12, APO3L, DR3LG, TWEAK and TNLG4A) encodes a cytokine that belongs to the tumor necrosis factor (TNF) ligand family. TNFSF12 is a ligand for the FN14/TWEAKR receptor. TNFSF12 has overlapping signaling functions with TNF but displays a much wider tissue distribution. TNFSF12, which exists in both membrane-bound and secreted forms, can induce apoptosis via multiple pathways of cell death in a cell type-specific manner. TNFSF12 is also found to promote proliferation and migration of endothelial cells and thus acts as a regulator of angiogenesis. In some embodiments, the human TNFSF12 gene refers to the gene described by NCBI Gene ID No. 8742. In some embodiments, the human TNFSF12 gene encodes the human TNFSF12 mRNA of NCBI Accession No. NM 003809. In some embodiments, the human TNFSF12 gene encodes one or more of the following human TNFSF12 mRNA variants: NCBI Accession No. NM_003809.3 and NR_037146.2. In some embodiments, the human TNFSF12 gene encodes one or more of the human TNFSF12 polypeptide isoforms: UniProt Accession No. 043508-1 and 043508-2.

The human mal, T-cell differentiation protein gene (also known as MAL, MVP17 and VIP17) encodes a highly hydrophobic integral membrane protein belonging to the MAL family of proteolipids. MAL has been localized to the endoplasmic reticulum of T-cells and is a candidate linker protein in T-cell signal transduction. In addition, this proteolipid is localized in compact myelin of cells in the nervous system and has been implicated in myelin biogenesis and/or function. MAL plays a role in the formation, stabilization and maintenance of glycosphingolipid-enriched membrane microdomains. Down-regulation of MAL gene has been associated with a variety of human epithelial malignancies. In some embodiments, the human MAL gene refers to the gene described by NCBI Gene ID No. 4118. In some embodiments, the human MAL gene encodes the human MAL mRNA of NCBI Accession No. NM_002371. In some embodiments, the human MAL gene encodes one or more of the following human MAL mRNA variants: NCBI Accession No. NM_002371.4, NM_022438.2, NM_022439.2 and NM_022440.2. In some embodiments, the human MAL gene encodes one or more of the human MAL polypeptide isoforms: UniProt Accession No. P21145-1, P21145-2, P21145-3 and P21145-4.

The human MYC associated factor X gene (also known as MAX and bHLHd4) encodes a member of the basic helix-loop-helix leucine zipper (bHLHZ) family of transcription factors. MAX is able to form homodimers and heterodimers with other family members, which include Mad, Mxi1 and Myc. Myc is an oncoprotein implicated in cell proliferation, differentiation and apoptosis. The homodimers and heterodimers compete for a common DNA target site (the E box) and rearrangement among these dimer forms provides a complex system of transcriptional regulation. Mutations of MAX have been reported to be associated with hereditary pheochromocytoma. In some embodiments, the human MAX gene refers to the gene described by NCBI Gene ID No. 4149. In some embodiments, the human MAX gene encodes the human MAX mRNA of NCBI Accession No. NM_002382. In some embodiments, the human MAX gene encodes one or more of the following human MAX mRNA variants: NCBI Accession No. NM_001271068.1, NM_001271069.1, NM_001320415.2, NM_002382.5, NM_145112.3, NM_145113.3, NM 145114.2, NM 197957.3, NR 073137.1, NR_073138.1, XM 011536773.3, XM_017021312.2, XM_017021313.1, XR_001750326.2, XR_001750327.2, XR_002957553.1, XR_943450.3, XR_943451.3 and XR_943452.3. In some embodiments, the human MAX gene encodes one or more of the human MAX polypeptide isoforms: UniProt Accession No. P61244-1, P61244-2, P61244-3, P61244-4, P61244-5 and P61244-6.

The human vascular endothelial growth factor B gene (also known as VEGFB, VRF and VEGFL) encodes a member of the PDGF (platelet-derived growth factor)/VEGF (vascular endothelial growth factor) family. The VEGF family members regulate the formation of blood vessels and are involved in endothelial cell physiology. VEGFB is a ligand for VEGFR-1 (vascular endothelial growth factor receptor 1) and NRP-1 (neuropilin-1). VEGF-B can regulate angiogenesis, redox and apoptosis by binding with VEGFR-1. VEGF-B is a potent survival factor for different types of cells by inhibiting apoptosis via suppressing the expression of BH3-only protein and other apoptotic/cell death-related genes. In some embodiments, the human VEGFB gene refers to the gene described by NCBI Gene ID No. 7423. In some embodiments, the human VEGFB gene encodes the human VEGFB mRNA of NCBI Accession No. NM_003377. In some embodiments, the human VEGFB gene encodes one or more of the following human VEGFB mRNA variants: NCBI Accession No. NM_001243733.2 and NM_003377.5. In some embodiments, the human VEGFB gene encodes one or more of the human VEGFB polypeptide isoforms: UniProt Accession No. P49765-1 and P49765-2.

In some embodiments, the at least one endogenous gene or gene product in the apoptosis signaling pathway is not Bax (NCBI Gene ID No. 581) or Bak (NCBI Gene ID No. 578)

In some embodiments, the cell comprises a modification that reduces or eliminates the activity of at least one endogenous gene or gene product selected from the list consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the cell comprises a modification that reduces or eliminates the activity of the ANGPT1 gene or gene product. In some embodiments, the cell comprises a modification that reduces or eliminates the activity of the ARHGEF7 gene or gene product. In some embodiments, the cell comprises a modification that reduces or eliminates the activity of the ARHGEF17 gene or gene product. In some embodiments, the cell comprises a modification that reduces or eliminates the activity of the BID gene or gene product. In some embodiments, the cell comprises a modification that reduces or eliminates the activity of the BNIP1 gene or gene product. In some embodiments, the cell comprises a modification that reduces or eliminates the activity of the C19orf2 gene or gene product. In some embodiments, the cell comprises a modification that reduces or eliminates the activity of the CHST11 gene or gene product. In some embodiments, the cell comprises a modification that reduces or eliminates the activity of the CUL1 gene or gene product. In some embodiments, the cell comprises a modification that reduces or eliminates the activity of the DAP3 gene or gene product. In some embodiments, the cell comprises a modification that reduces or eliminates the activity of the DYNLL1 gene or gene product. In some embodiments, the cell comprises a modification that reduces or eliminates the activity of the FIS1 gene or gene product. In some embodiments, the cell comprises a modification that reduces or eliminates the activity of the HMGB2 gene or gene product. In some embodiments, the cell comprises a modification that reduces or eliminates the activity of the IGFBP3 gene or gene product. In some embodiments, the cell comprises a modification that reduces or eliminates the activity of the NRG1 gene or gene product. In some embodiments, the cell comprises a modification that reduces or eliminates the activity of the TNFRSF10C gene or gene product. In some embodiments, the cell comprises a modification that reduces or eliminates the activity of the TNFSF12 gene or gene product. In some embodiments, the cell comprises a modification that reduces or eliminates the activity of the MAL gene or gene product. In some embodiments, the cell comprises a modification that reduces or eliminates the activity of the MAX gene or gene product. In some embodiments, the cell comprises a modification that reduces or eliminates the activity of the VEGFB gene or gene product. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell.

In some embodiments, the cell comprises modifications that reduce or eliminate the activity of at least two endogenous gene or gene product selected from the group consisting of the ANGPT1, ARHGEF7, ARHGEF17, BID, BNIP1. C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the cell comprises modifications that reduce or eliminate the activity of at least two endogenous gene or gene product selected from the group consisting of the ARHGEF7, ARHGEF17, BNIP1, C19orf2, CHST11, DYNLL1, IGFBP3, MAX, and VEGFB gene or gene product. In some embodiments, the cell comprises a first modification that reduces or eliminates the activity of the activity of the ARHGEF7 gene or gene product and a second modification that reduces or eliminates the activity of a second endogenous gene or gene product selected from the group consisting of the ARHGEF17, BNIP1, C19orf2, CHST11, DYNLL1, IGFBP3, MAX, and VEGFB gene or gene product. In some embodiments, the cell comprises a first modification that reduces or eliminates the activity of the activity of the ARHGEF17 gene or gene product and a second modification that reduces or eliminates the activity of a second endogenous gene or gene product selected from the group consisting of the ARHGEF7, BNIP1, C19orf2, CHST11, DYNLL1, IGFBP3, MAX, and VEGFB gene or gene product. In some embodiments, the cell comprises a first modification that reduces or eliminates the activity of the activity of the BNIP1 gene or gene product and a second modification that reduces or eliminates the activity of a second endogenous gene or gene product selected from the group consisting of the ARHGEF7, ARHGEF17, C19orf2, CHST11, DYNLL1, IGFBP3, MAX, and VEGFB gene or gene product. In some embodiments, the cell comprises a first modification that reduces or eliminates the activity of the activity of the C19orf2 gene or gene product and a second modification that reduces or eliminates the activity of a second endogenous gene or gene product selected from the group consisting of the ARHGEF7, ARHGEF17, BNIP1, CHST11, DYNLL1, IGFBP3, MAX, and VEGFB gene or gene product. In some embodiments, the cell comprises a first modification that reduces or eliminates the activity of the activity of the CHST11 gene or gene product and a second modification that reduces or eliminates the activity of a second endogenous gene or gene product selected from the group consisting of the ARHGEF7, ARHGEF17, BNIP1, C19orf2, DYNLL1, IGFBP3, MAX, and VEGFB gene or gene product. In some embodiments, the cell comprises a first modification that reduces or eliminates the activity of the DYNLL1 gene or gene product and a second modification that reduces or eliminates the activity of a second endogenous gene or gene product selected from the group consisting of the ARHGEF7, ARHGEF17, BNIP1, C19orf2, CHST11, IGFBP3, MAX, and VEGFB gene or gene product. In some embodiments, the cell comprises a first modification that reduces or eliminates the activity of the DYNLL1 gene or gene product and a second modification that reduces or eliminates the activity of the MAX gene or gene product. In some embodiments, the cell comprises a first modification that reduces or eliminates the activity of the DYNLL1 gene or gene product and a second modification that reduces or eliminates the activity of the ANGPT1 gene or gene product. In some embodiments, the cell comprises a first modification that reduces or eliminates the activity of the DYNLL1 gene or gene product and a second modification that reduces or eliminates the activity of the ARHGEF7 gene or gene product. In some embodiments, the cell comprises a first modification that reduces or eliminates the activity of the DYNLL1 gene or gene product and a second modification that reduces or eliminates the activity of the ARHGEF17 gene or gene product. In some embodiments, the cell comprises a first modification that reduces or eliminates the activity of the DYNLL1 gene or gene product and a second modification that reduces or eliminates the activity of the BID gene or gene product. In some embodiments, the cell comprises a first modification that reduces or eliminates the activity of the DYNLL1 gene or gene product and a second modification that reduces or eliminates the activity of the BNIP1 gene or gene product. In some embodiments, the cell comprises a first modification that reduces or eliminates the activity of the DYNLL1 gene or gene product and a second modification that reduces or eliminates the activity of the C19orf2 gene or gene product. In some embodiments, the cell comprises a first modification that reduces or eliminates the activity of the DYNLL1 gene or gene product and a second modification that reduces or eliminates the activity of the CHST11 gene or gene product. In some embodiments, the cell comprises a first modification that reduces or eliminates the activity of the DYNLL1 gene or gene product and a second modification that reduces or eliminates the activity of the CUL1 gene or gene product. In some embodiments, the cell comprises a first modification that reduces or eliminates the activity of the DYNLL1 gene or gene product and a second modification that reduces or eliminates the activity of the DAP3 gene or gene product. In some embodiments, the cell comprises a first modification that reduces or eliminates the activity of the DYNLL1 gene or gene product and a second modification that reduces or eliminates the activity of the FIS1 gene or gene product. In some embodiments, the cell comprises a first modification that reduces or eliminates the activity of the DYNLL1 gene or gene product and a second modification that reduces or eliminates the activity of the HMGB2 gene or gene product. In some embodiments, the cell comprises a first modification that reduces or eliminates the activity of the DYNLL1 gene or gene product and a second modification that reduces or eliminates the activity of the IGFBP3 gene or gene product. In some embodiments, the cell comprises a first modification that reduces or eliminates the activity of the DYNLL1 gene or gene product and a second modification that reduces or eliminates the activity of the NRG1 gene or gene product. In some embodiments, the cell comprises a first modification that reduces or eliminates the activity of the DYNLL1 gene or gene product and a second modification that reduces or eliminates the activity of the TNFRSF10C gene or gene product. In some embodiments, the cell comprises a first modification that reduces or eliminates the activity of the DYNLL1 gene or gene product and a second modification that reduces or eliminates the activity of the TNFSF12 gene or gene product. In some embodiments, the cell comprises a first modification that reduces or eliminates the activity of the DYNLL1 gene or gene product and a second modification that reduces or eliminates the activity of the MAL gene or gene product. In some embodiments, the cell comprises a first modification that reduces or eliminates the activity of the DYNLL1 gene or gene product and a second modification that reduces or eliminates the activity of the VEGFB gene or gene product. In some embodiments, the cell comprises a first modification that reduces or eliminates the activity of the activity of the IGFBP3 gene or gene product and a second modification that reduces or eliminates the activity of a second endogenous gene or gene product selected from the group consisting of the ARHGEF7, ARHGEF17, BNIP1, C19orf2, CHST11, DYNLL1, MAX, and VEGFB gene or gene product. In some embodiments, the cell comprises a first modification that reduces or eliminates the activity of the activity of the MAX gene or gene product and a second modification that reduces or eliminates the activity of a second endogenous gene or gene product selected from the group consisting of the ARHGEF7, ARHGEF17, BNIP1, C19orf2, CHST11, DYNLL1, IGFBP3, and VEGFB gene or gene product. In some embodiments, the cell comprises a first modification that reduces or eliminates the activity of the activity of the MAX gene or gene product and a second modification that reduces or eliminates the activity of the DYNLL1 gene or gene product. In some embodiments, the cell comprises a first modification that reduces or eliminates the activity of the activity of the VEGFB gene or gene product and a second modification that reduces or eliminates the activity of a second endogenous gene or gene product selected from the group consisting of the ARHGEF7, ARHGEF17, BNIP1, C19orf2, CHST11, DYNLL1, IGFBP3, and MAX gene or gene product. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell.

In some embodiments, the cell comprises modifications that reduce or eliminate the activity of at least three endogenous gene or gene product selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the cell comprises modifications that reduce or eliminate the activity of at least three endogenous gene or gene product selected from the group consisting of the ARHGEF7, ARHGEF17, BNIP1, C19orf2, CHST11, DYNLL1, IGFBP3, MAX, and VEGFB gene or gene product. In some embodiments, the cell comprises modifications that reduce or eliminate the activity of the DYNLL1 and MAX genes or gene products and a third gene or gene product selected from the group consisting of the ANGPT1, ARHGEF7, ARHGEF17, BID, BNIP1, C19orf2, CHST11, CUL1 DAP3, FIS1, HMGB2, IGFBP3, NRG1, TNFRSF10C, TNFSF12, MAL, and VEGFB gene or gene product. In some embodiments, the cell comprises modifications that reduce or eliminate the activity of the DYNLL1 and MAX genes or gene products and a third gene or gene product selected from the group consisting of the ARHGEF7, ARHGEF17, BNIP1, C19orf2, CHST11, IGFBP3, and VEGFB gene or gene product. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell.

In some embodiments, the cell comprises modifications that reduce or eliminate the activity of at least four, five or six endogenous gene or gene product selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product. In some embodiments, the cell comprises modifications that reduce or eliminate the activity of at least four, five or six endogenous gene or gene product selected from the group consisting of the ARHGEF7, ARHGEF17, BNIP1, C19orf2, CHST11, DYNLL1, IGFBP3, MAX, and VEGFB gene or gene product. In some embodiments, the cell comprises modifications that reduce or eliminate the activity of the DYNLL1 and MAX genes or gene products and at least a third and fourth gene or gene product selected from the group consisting of the ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, and VEGFB gene or gene product. In some embodiments, the cell comprises modifications that reduce or eliminate the activity of the DYNLL1 and MAX genes or gene products and at least a third and fourth gene or gene product selected from the group consisting of the ARHGEF7, ARHGEF17, BNIP1, C19orf2, CHST11, IGFBP3, and VEGFB gene or gene product. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell.

In some embodiments, the modifications that reduce or eliminate the activity of the endogenous gene or gene product is a mutation in the gene. In some embodiments, the mutation is a loss-of-function mutation. A “loss-of-function mutation” in a gene refers to a genetic manipulation or mutation (e.g., a substitution, deletion, insertion, duplication, frameshift, or translocation) in a gene that reduces or eliminates one or more functions of the corresponding gene product. In some embodiments, the loss-of-function mutation is a null mutation that eliminates one or more functions of the corresponding gene product, e.g., a deletion that removes some or all of the coding sequence. In some embodiments, the mutation is a missense mutation. In some embodiments, the mutation is a nonsense mutation. In some embodiments, the mutation is a frameshift mutation. In some embodiments, the mutation is a deletion. In some embodiments, the mutation that reduces or eliminates the activity of the endogenous gene or gene product is present in all copies of the genome. In some embodiments, the mutation that reduces or eliminates the activity of the endogenous gene or gene product is present in less than all copies of the genome.

A skilled artisan is aware that a eukaryotic cell (e.g., a mammalian cell, such as an HEK293 cell) comprises more than one copy of a gene. Consequently, a modification that reduces or eliminates the activity of the endogenous gene or gene product can be a mutation or mutations that affects one, more than one or all copies of the gene. In one embodiment, the mutation is a heterozygous mutation that does not affect all copies of the gene. In one embodiment, the mutation is a heterozygous mutation that affects one copy of the gene. In one embodiment, the mutation is a heterozygous mutation that affects two copies of the gene. In one embodiment, the mutation is a heterozygous mutation that affects all but one copy of the gene. In one embodiment, the mutation is a homozygous mutation that affects all copies of the gene. In some embodiments, the mutation is a heterozygous mutation (e.g., deletion) in an endogenous gene selected from the group consisting of the ARHGEF7, ARHGEF17, BNIP1, C19orf2, CHST11, DYNLL1, IGFBP3, MAX, and VEGFB. In some embodiments, the mutation is a heterozygous mutation (e.g., deletion) in DYNLL1. In some embodiments, the mutation is a heterozygous mutation (e.g., deletion) in MAX, In some embodiments, the mutation is a heterozygous mutation in MAX, In some embodiments, the mutation is a homozygous mutation (e.g., deletion) in an endogenous gene selected from the group consisting of the ARHGEF7, ARHGEF17, BNIP1, C19orf2, CHST11, DYNLL1, IGFBP3, MAX, and VEGFB. In some embodiments, the mutation is a homozygous mutation (e.g., deletion) in DYNLL1. In some embodiments, the mutation is a homozygous mutation (e.g., deletion) in MAX,

A skilled artisan is aware of technologies that can be used to introduce mutations (e.g., loss-of-function mutations) into the genetic material of a cell (e.g., HEK293 cell). These include, without limitation, zinc finger nuclease technology, CRISPR/Cas9 technology, and TALEN technology to introduce targeted mutations and chemical and insertional mutagenesis with selection to identify random mutations. Any technology known to a skilled artisan can be used to introduce a modification (e.g., loss-of function mutation) that reduces or eliminates the activity of an endogenous gene or gene product selected from the group consisting of the ARHGEF7, ARHGEF17, BNIP1, C19orf2, CHST11, DYNLL1, IGFBP3, MAX, and VEGFB gene or gene product.

In some embodiments, the modifications that reduce or eliminate the activity of the endogenous gene or gene product is an inhibitory nucleic acid capable of reducing or eliminating the activity of a gene or gene product. A skilled artisan is aware of inhibitory nucleic acid-based technologies that can be used to reduce or eliminate the activity of an endogenous gene or gene product in a cell (e.g., HEK293 cell). In some embodiments, the inhibitory nucleic acid comprises small interfering RNA (siRNA), microRNA (miRNA) or short hairpin RNA (shRNA). In some embodiments, the inhibitory nucleic acid comprises an antisense RNA. In some embodiments, the inhibitory nucleic acid comprises a morpholino oligomer. Tools for designing an inhibitory nucleic acid that can target a specific gene are well known in the art. The sequence of inhibitory nucleic acid molecules for targeting a gene of interest are also readily available to the skilled artisan, for example, at The Genetic Perturbation Platform, formerly known as the RNA interference (RNAi) Platform accessible at portals.broadinstitute.org/gpp/public/. Additional sources of information and material include commercial services, such as Invitrogen. A skilled artisan understands that inhibitory nucleic acid molecules can be introduced into a cell by direct transfection or by expression from a vector. Any technology known to a skilled artisan can be used to introduce a modification (e.g., inhibitory nucleic acid) that reduces or eliminates the activity of an endogenous gene or gene product selected from the group consisting of the ARHGEF7, ARHGEF17, BNIP1, C19orf2, CHST11, DYNLL1, IGFBP3. MAX, and VEGFB gene or gene product.

In some embodiments, the modification that reduces or eliminates the activity of an endogenous gene or gene product eliminates an mRNA transcript encoded by the gene. In some embodiments, the modification that reduces or eliminates the activity of an endogenous gene or gene product reduces the level of an mRNA encoded by the gene by at least about 50%, 60%, 70%, 80%, 90% or 95%.

In some embodiments, the modification that reduces or eliminates the activity of an endogenous gene or gene product eliminates a polypeptide encoded the gene. In some embodiments, the modification that reduces or eliminates the activity of an endogenous gene or gene product reduces the level of a protein encoded by the gene by at least about 50%, 60%, 70%, 80%, 90% or 95%.

In some embodiments, the modification that reduces or eliminates the activity of an endogenous gene or gene product is a missense mutation that reduces the activity of a protein encoded by the gene by at least about 50%, 60%, 70%, 80%, 90% or 95%.

In some embodiments, a cell described herein further comprises one or more polynucleotides encoding a recombinant virus. Transfection based recombinant virus particle production systems are known to the skilled artisan. See, e.g., Reiser et al., Gene Ther 7(11):910-3 (2000); Dull et al., J Virol. 72(11): 8463-8471 (1998): Hoffmann et al., PNAS 97 (11) 6108-6113 (2000); Milian et al., Vaccine 35(26): 3423-3430 (2017), each of which is incorporated herein by reference in its entirety. In some embodiments, the recombinant viral particle is a recombinant Dengue virus, a recombinant Ebola virus, a recombinant human papillomavirus (HPV), a recombinant human immunodeficiency virus (HIV), a recombinant adeno-associated virus (AAV), a recombinant lentivirus, a recombinant influenza virus, a recombinant vesicular stomatitis virus (VSV), a recombinant poliovirus, a recombinant adenovirus, a recombinant retrovirus, a recombinant vaccinia, a recombinant reovirus, a recombinant measles, a recombinant Newcastle disease virus (NDV), a recombinant herpes zoster virus (HZV), a recombinant herpes simplex virus (HSV), or a recombinant baculovirus. In some embodiments, the recombinant viral particle is a recombinant adeno-associated virus (AAV), a recombinant lentivirus, or a recombinant influenza virus. In some embodiments, the recombinant viral particle is a recombinant lentivirus. In some embodiments, the recombinant viral particle is a recombinant influenza virus. In some embodiments, the recombinant viral particle is a recombinant baculovirus. In some embodiments, the recombinant viral particle is a recombinant adeno-associated virus (AAV).

In some embodiments, a cell described herein further comprises one or more polynucleotides encoding a recombinant AAV. In some embodiments, a cell described herein further comprises one or more polynucleotides encoding at least one of: an rAAV genome to be packaged, adenovirus helper functions necessary for packaging, an AAV rep protein sufficient for packaging, and an AAV cap protein sufficient for packaging. In some embodiments, a cell described herein further comprises one or more polynucleotides encoding: an rAAV genome to be packaged, adenovirus helper functions necessary for packaging, an AAV rep protein sufficient for packaging, and an AAV cap protein sufficient for packaging.

In some embodiments, a cell described herein further comprises one or more polynucleotides encoding a recombinant polypeptide. In some embodiment, the recombinant polypeptide is an antibody or antibody fragment. In some embodiments, the antibody is a chimeric, human or humanized antibody. In some embodiments, the antibody is an IgG antibody. In some embodiments, the recombinant polypeptide is a fusion protein. In some embodiments, the fusion protein comprises an antibody or fragment thereof. In some embodiments, the fusion protein comprises an Fc region. In some embodiments, the fusion protein comprises an antigen-binding antibody fragment.

### Cell Banks

In one aspect, the disclosure provides a cell bank comprising a plurality of cells described herein. In some embodiments, the cell bank comprises a plurality of cryopreserved cells. In some embodiments, the cells are suitable for recombinant virus (e.g., recombinant AAV) production. In some embodiments, the cells are suitable for recombinant polypeptide production. In some embodiments, the cells are HEK293 cells. In some embodiments, the cells are HEK293 derived cells. In some embodiments, the cells are suspension adapted HEK293 cells. In some embodiments, the cells are suspension adapted HEK293 derived cells.

In one aspect, the disclosure provides a method of constructing a cell bank comprising cryopreserving a composition comprising a plurality of cells described herein. In some embodiments, the cells are suitable for recombinant virus (e.g., recombinant AAV) production. In some embodiments, the cells are suitable for recombinant polypeptide production. In some embodiments, the cells are HEK293 cells. In some embodiments, the cells are HEK293 derived cells. In some embodiments, the cells are suspension adapted HEK293 cells. In some embodiments, the cells are suspension adapted HEK293 derived cells.

### Cell Cultures

In one aspect, the disclosure provides a cell culture comprising a plurality of cells described herein. In some embodiments, the cell culture comprises a plurality of cells described herein and a suitable medium. In some embodiments, the cell culture is a suspension culture. In some embodiments, the cells are capable of producing a recombinant virus (e.g., recombinant AAV). In some embodiments, the cells comprise one or more polynucleotides encoding at least one of: an rAAV genome to be packaged, adenovirus helper functions necessary for packaging, an AAV rep protein sufficient for packaging, and an AAV cap protein sufficient for packaging. In some embodiments, the cells comprise one or more polynucleotides encoding: an rAAV genome to be packaged, adenovirus helper functions necessary for packaging, an AAV rep protein sufficient for packaging, and an AAV cap protein sufficient for packaging. In some embodiments, the cells are capable of producing a recombinant polypeptide (e/g/, antibody). In some embodiments, the cells comprise one or more polynucleotides encoding an antibody or antigen-binding antibody fragment. In some embodiments, the cells are HEK293 cells. In some embodiments, the cells are HEK293 derived cells. In some embodiments, the cells are suspension adapted HEK293 cells. In some embodiments, the cells are suspension adapted HEK293 derived cells.

### Methods of Producing a Recombinant Viral Particle

In one aspect, the disclosure provides a method of producing a recombinant virus (e.g., recombinant adeno-associated virus (rAAV)) particles in a host cell described herein. In some embodiments, the method further comprises recovering the virus (e.g., rAAV) particles.

In some embodiments, the disclosure provides a method of producing a recombinant virus particle (e.g., rAAV), comprising (a) providing a cell culture comprising a plurality of cells described herein suitable for producing the recombinant virus particle; (b) transfecting the cells with one or more polynucleotides containing genes necessary for producing the recombinant virus particle; and (c) maintaining the cell culture comprising the transfected cells under conditions that allow the production of the recombinant virus particle. Transfection based recombinant virus particle production systems are known to the skilled artisan. See, e.g., Reiser et al., Gene Ther 7(11):910-3 (2000); Dull et al., J Virol. 72(11): 8463-8471 (1998); Hoffnann et al., PNAS 97 (11) 6108-6113 (2000); Milian et al., Vaccine 35(26): 3423-3430 (2017), each of which is incorporated herein by reference in its entirety. In some embodiments, the recombinant viral particle is a recombinant Dengue virus, a recombinant Ebola virus, a recombinant human papillomavirus (HPV), a recombinant human immunodeficiency virus (HIV), a recombinant adeno-associated virus (AAV), a recombinant lentivirus, a recombinant influenza virus, a recombinant vesicular stomatitis virus (VSV), a recombinant poliovirus, a recombinant adenovirus, a recombinant retrovirus, a recombinant vaccinia, a recombinant reovirus, a recombinant measles, a recombinant Newcastle disease virus (NDV), a recombinant herpes zoster virus (HZV), a recombinant herpes simplex virus (HSV), or a recombinant baculovirus. In some embodiments, the recombinant viral particle is a recombinant adeno-associated virus (AAV), a recombinant lentivirus, or a recombinant influenza virus. In some embodiments, the recombinant viral particle is a recombinant lentivirus. In some embodiments, the recombinant viral particle is a recombinant influenza virus. In some embodiments, the recombinant viral particle is a recombinant baculovirus. In some embodiments, the recombinant viral particle is a recombinant adeno-associated virus (AAV).

In some embodiments, the disclosure provides a method of producing rAAV particles, comprising (a) providing a cell culture comprising a plurality of cells described herein capable of producing rAAV; and (b) maintaining the cell culture under conditions that allow production of the rAAV particles. In some embodiments, the cell capable of producing rAAV has been transfected with one or more polynucleotides encoding at least one of an rAAV genome to be packaged, adenovirus helper functions necessary for packaging, an AAV rep protein sufficient for packaging, and an AAV cap protein sufficient for packaging. In some embodiments, the cell capable of producing rAAV has been transfected with one or more polynucleotides encoding an rAAV genome to be packaged, adenovirus helper functions necessary for packaging, an AAV rep protein sufficient for packaging, and an AAV cap protein sufficient for packaging. In some embodiments, the adenovirus helper functions comprise at least one of an adenovirus E4 gene, E2a gene, and VA gene. In some embodiments, the adenovirus helper functions comprise an adenovirus E4 gene, E2a gene, and VA gene. In some embodiments, the polynucleotide encoding the adenovirus helper functions comprises pAD Delta F6. In some embodiments, the polynucleotide encoding the adenovirus helper functions comprises a helper plasmid disclosed in WO 2023/060113, such as Helper #5, which is incorporated herein by reference in its entirety. In some embodiments, the polynucleotide encoding the adenovirus helper functions also encodes the AAV rep protein and the AAV cap protein. In some embodiments, the polynucleotide encoding the adenovirus helper functions, AAV rep protein and AAV cap protein comprises a helper/rep/cap plasmid, such as pHRC #7 and pHRD #8, disclosed in PCT/US2024/023368, filed Apr. 5, 2024, which is incorporated herein by reference in its entirety.

In some embodiments, the disclosure provides a method for producing rAAV particles, comprising culturing a cell described herein capable of producing rAAV particles under conditions that allow the production of the rAAV particles. In some embodiments, the cell capable of producing rAAV has been transfected with one or more polynucleotides encoding at least one of an rAAV genome to be packaged, adenovirus helper functions necessary for packaging, an AAV rep protein sufficient for packaging, and an AAV cap protein sufficient for packaging. In some embodiments, the cell capable of producing rAAV has been transfected with one or more polynucleotides encoding an rAAV genome to be packaged, adenovirus helper functions necessary for packaging, an AAV rep protein sufficient for packaging, and an AAV cap protein sufficient for packaging. In some embodiments, the adenovirus helper functions comprise at least one of an adenovirus E4 gene, E2a gene, and VA gene. In some embodiments, the adenovirus helper functions comprise an adenovirus E4 gene, E2a gene, and VA gene. In some embodiments, the polynucleotide encoding the adenovirus helper functions comprises pAD Delta F6. In some embodiments, the polynucleotide encoding the adenovirus helper functions comprises a helper plasmid disclosed in WO 2023/060113, such as Helper #5, which is incorporated herein by reference in its entirety. In some embodiments, the polynucleotide encoding the adenovirus helper functions also encodes the AAV rep protein and the AAV cap protein. In some embodiments, the polynucleotide encoding the adenovirus helper functions, AAV rep protein and AAV cap protein comprises a helper/rep/cap plasmid, such as pHRC #7 and pHRD #8, disclosed in PCT/US2024/023368, filed Apr. 5, 2024, which is incorporated herein by reference in its entirety.

In some embodiments, the disclosure provides a method of increasing the production of rAAV particles, comprising (a) providing a cell culture comprising a plurality of cells described herein capable of producing rAAV; and (b) maintaining the cell culture under conditions that allow production of the rAAV particles. In some embodiments, the cell capable of producing rAAV has been transfected with one or more polynucleotides encoding at least one of an rAAV genome to be packaged, adenovirus helper functions necessary for packaging, an AAV rep protein sufficient for packaging, and an AAV cap protein sufficient for packaging. In some embodiments, the cell capable of producing rAAV has been transfected with one or more polynucleotides encoding an rAAV genome to be packaged, adenovirus helper functions necessary for packaging, an AAV rep protein sufficient for packaging, and an AAV cap protein sufficient for packaging. In some embodiments, the adenovirus helper functions comprise at least one of an adenovirus E4 gene, E2a gene, and VA gene. In some embodiments, the adenovirus helper functions comprise an adenovirus E4 gene, E2a gene, and VA gene. In some embodiments, the polynucleotide encoding the adenovirus helper functions comprises pAD Delta F6. In some embodiments, the polynucleotide encoding the adenovirus helper functions comprises a helper plasmid disclosed in WO 2023/060113, such as Helper #5, which is incorporated herein by reference in its entirety. In some embodiments, the polynucleotide encoding the adenovirus helper functions also encodes the AAV rep protein and the AAV cap protein. In some embodiments, the polynucleotide encoding the adenovirus helper functions, AAV rep protein and AAV cap protein comprises a helper/rep/cap plasmid, such as pHRC #7 and pHRD #8, disclosed in PCT/US2024/023368, filed Apr. 5, 2024, which is incorporated herein by reference in its entirety.

In some embodiments, the disclosure provides a method of increasing the production of rAAV particles, comprising culturing a plurality of cells described herein capable of producing rAAV particles under conditions that allow the production of the rAAV particles. In some embodiments, the cell capable of producing rAAV has been transfected with one or more polynucleotides encoding at least one of an rAAV genome to be packaged, adenovirus helper functions necessary for packaging, an AAV rep protein sufficient for packaging, and an AAV cap protein sufficient for packaging. In some embodiments, the cell capable of producing rAAV has been transfected with one or more polynucleotides encoding an rAAV genome to be packaged, adenovirus helper functions necessary for packaging, an AAV rep protein sufficient for packaging, and an AAV cap protein sufficient for packaging. In some embodiments, the adenovirus helper functions comprise at least one of an adenovirus E4 gene, E2a gene, and VA gene. In some embodiments, the adenovirus helper functions comprise an adenovirus E4 gene, E2a gene, and VA gene. In some embodiments, the polynucleotide encoding the adenovirus helper functions comprises pAD Delta F6. In some embodiments, the polynucleotide encoding the adenovirus helper functions comprises a helper plasmid disclosed in WO 2023/060113, such as Helper #5, which is incorporated herein by reference in its entirety. In some embodiments, the polynucleotide encoding the adenovirus helper functions also encodes the AAV rep protein and the AAV cap protein. In some embodiments, the polynucleotide encoding the adenovirus helper functions, AAV rep protein and AAV cap protein comprises a helper/rep/cap plasmid, such as pHRC #7 and pHRD #8, disclosed in PCT/US2024/023368, filed Apr. 5, 2024, which is incorporated herein by reference in its entirety.

In some embodiments, the disclosure provides a method of producing rAAV particles, comprising (a) providing a cell culture comprising a plurality of cells; (b) introducing into the cells one or more polynucleotides encoding at least one of (i) an rAAV genome to be packaged, (ii) adenovirus helper functions necessary for packaging, (iii) an AAV rep protein sufficient for packaging, and (iv) an AAV cap protein sufficient for packaging; and (c) maintaining the cell culture under conditions that allow production of the rAAV particles. In some embodiments, the method comprises introducing into the cell one or more polynucleotides encoding (i) an rAAV genome to be packaged, (ii) adenovirus helper functions necessary for packaging, (iii) an AAV rep protein sufficient for packaging, and (iv) an AAV cap protein sufficient for packaging. In some embodiments, the introducing one or more polynucleotides into the cell is by transfection. In some embodiments, the adenovirus helper functions comprise at least one of an adenovirus E4 gene, E2a gene, and VA gene. In some embodiments, the adenovirus helper functions comprise an adenovirus E4 gene, E2a gene, and VA gene. In some embodiments, the polynucleotide encoding the adenovirus helper functions comprises pAD Delta F6. In some embodiments, the polynucleotide encoding the adenovirus helper functions comprises a helper plasmid disclosed in WO 2023/060113, such as Helper #5, which is incorporated herein by reference in its entirety. In some embodiments, the polynucleotide encoding the adenovirus helper functions also encodes the AAV rep protein and the AAV cap protein. In some embodiments, the polynucleotide encoding the adenovirus helper functions, AAV rep protein and AAV cap protein comprises a helper/rep/cap plasmid, such as pHRC #7 and pHRD #8, disclosed in PCT/US2024/023368, filed Apr. 5, 2024, which is incorporated herein by reference in its entirety.

In some embodiments, the disclosure provides a method of increasing the production of rAAV particles, comprising (a) providing a cell culture comprising a plurality of cells: (b) introducing into the cells one or more polynucleotides encoding at least one of (i) an rAAV genome to be packaged, (ii) adenovirus helper functions necessary for packaging, (iii) an AAV rep protein sufficient for packaging, and (iv) an AAV cap protein sufficient for packaging; and (c) maintaining the cell culture under conditions that allow production of the rAAV particles. In some embodiments, the method comprises introducing into the cell one or more polynucleotides encoding (i) an rAAV genome to be packaged, (ii) adenovirus helper functions necessary for packaging, (iii) an AAV rep protein sufficient for packaging, and (iv) an AAV cap protein sufficient for packaging. In some embodiments, the introducing one or more polynucleotides into the cell is by transfection. In some embodiments, the adenovirus helper functions comprise at least one of an adenovirus E4 gene, E2a gene, and VA gene. In some embodiments, the adenovirus helper functions comprise an adenovirus E4 gene, E2a gene, and VA gene. In some embodiments, the polynucleotide encoding the adenovirus helper functions comprises pAD Delta F6. In some embodiments, the polynucleotide encoding the adenovirus helper functions comprises a helper plasmid disclosed in WO 2023/060113, such as Helper #5, which is incorporated herein by reference in its entirety. In some embodiments, the polynucleotide encoding the adenovirus helper functions also encodes the AAV rep protein and the AAV cap protein. In some embodiments, the polynucleotide encoding the adenovirus helper functions, AAV rep protein and AAV cap protein comprises a helper/rep/cap plasmid, such as pHRC #7 and pHRD #8, disclosed in PCT/US2024/023368, filed Apr. 5, 2024, which is incorporated herein by reference in its entirety.

In some embodiments, the maintaining the cell culture or culturing the cell under conditions that allow production of the rAAV particles is for between about 2 days and about 10 days, between about 2 days and about 15 days, or between about 5 days and 14 days. In some embodiments, the maintaining the cell culture or culturing the cell under conditions that allow production of the rAAV particles is for about 2 days, about 3 days, about 4 days, about 5 days, about 6 days, or about 7 days. In some embodiments, the maintaining the cell culture or culturing the cell under conditions that allow production of the rAAV particles is for about 2 days, about 3 days, about 4 days, about 5 days, about 6 days, or about 7 days. In some embodiments, the maintaining the cell culture or culturing the cell under conditions that allow production of the rAAV particles is for about 5 days.

In some embodiments, the cell is a mammalian cell. In some embodiments, the cell is an insect cell. In some embodiments, the cell is a HEK293 cell, HEK derived cell, CHO cell, CHO derived cell, HeLa cell, SF-9 cell, BHK cell, Vero cell, or PerC6 cell. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell.

In some embodiments, the cell culture is a suspension culture or an adherent culture. In some embodiments, the cell culture is a suspension culture.

In some embodiments, the cell culture has a volume between about 50 liters and about 20,000 liters. In some embodiments, the cell culture has a volume between about 100 liters and 5,000 liters. In some embodiments, the cell culture has a volume between about 100 liters and 2,500 liters. In some embodiments, the cell culture has a volume between about 100 liters and 1,500 liters. In some embodiments, the cell culture has a volume between about 200 liters and 5,000 liters. In some embodiments, the cell culture has a volume between about 200 liters and 2,500 liters. In some embodiments, the cell culture has a volume between about 200 liters and 1,500 liters. In some embodiments, the cell culture has a volume between about 250 liters and 5,000 liters. In some embodiments, the cell culture has a volume between about 250 liters and 2.500 liters. In some embodiments, the cell culture has a volume between about 250 liters and 1,500 liters.

In some embodiments, the method further comprises recovering the rAAV particles.

In some embodiments, a method described herein produces more rAAV particles measured as GC/ml than a reference method. In some embodiments, the reference method uses a host cell that does not comprise at least one modification that reduces or eliminates the activity of at least one endogenous gene or gene product in the apoptosis signaling pathway. In some embodiments, the reference method uses a HEK293 host cell that does not comprise at least one modification that reduces or eliminates the activity of at least one endogenous gene or gene product in the apoptosis signaling pathway. In some embodiments, the method described herein produces at least about 10% more rAAV particles measured as GC/ml than the reference method. In some embodiments, the method described herein produces at least about 20% more rAAV particles measured as GC/ml than the reference method. In some embodiments, the method described herein produces at least about 30% more rAAV particles measured as GC/ml than the reference method. In some embodiments, the method described herein produces at least about 40% more rAAV particles measured as GC/ml than the reference method. In some embodiments, the method described herein produces at least about 50% more rAAV particles measured as GC/ml than the reference method. In some embodiments, the method described herein produces at least about 70% more rAAV particles measured as GC/ml than the reference method. In some embodiments, the method described herein produces at least about 90% more rAAV particles measured as GC/ml than the reference method. In some embodiments, the method described herein produces at least about twice as many rAAV particles measured as GC/ml than the reference method. In some embodiments, the method produces at least about three times as many rAAV particles measured as GC/ml than the reference method. In some embodiments, the method produces at least about four times as many rAAV particles measured as GC/ml than the reference method.

In some embodiments, the method produces a population of rAAV particles comprising more full capsids than a reference method. In some embodiments, the reference method uses a host cell that does not comprise at least one modification that reduces or eliminates the activity of at least one endogenous gene or gene product in the apoptosis signaling pathway. In some embodiments, the reference method uses a HEK293 host cell that does not comprise at least one modification that reduces or eliminates the activity of at least one endogenous gene or gene product in the apoptosis signaling pathway.

In some embodiments, the rAAV particles comprise a capsid protein of the AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 serotype. In some embodiments, the rAAV particles comprise a capsid protein of the AAV8, AAV9, AAV.rh10, AAV.rh20, AAV.rh39, AAV.Rh74, AAV.RHM4-1, AAV.hu32, or AAV.hu37 serotype. In some embodiments, the rAAV particles comprise a capsid protein of the AAV8 serotype. In some embodiments, the rAAV particles comprise a capsid protein of the AAV9 serotype.

In some embodiments, the rAAV particle comprises a transgene encoding a gene product. In some embodiments, the gene product is a polypeptide or a double stranded RNA molecule. In some embodiments, the gene product is a polypeptide. In some embodiments, the transgene encodes an antibody or antigen-binding fragment thereof, fusion protein, Fc-fusion polypeptide, immunoadhesin, immunoglobulin, engineered protein, protein fragment or enzyme. In some embodiments, the transgene comprises a regulatory element operatively connected to a polynucleotide encoding the gene product.

In some embodiments, the gene product is anti-VEGF Fab, anti-kallikrein antibody, anti-TNF antibody, microdystrophin, minidystrophin, iduronidase (IDUA), iduronate 2-sulfatase (IDS), low-density lipoprotein receptor (LDLR), tripeptidyl peptidase 1 (TPP1), or non-membrane associated splice variant of VEGF receptor 1 (sFlt-1). In some embodiments, the gene product is an gamma-sarcoglycan. Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterasc 6B (PDE6B), ornithine carbanioyltransferase 90TC), survival motor neuron (SMN1), survival motor neuron (SMN2), neurturin (NRTN). Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fe fusion. In some embodiments, the gene product is a dystrophin or a microdystrophin. In some embodiments, the gene product is a microRNA.

In some embodiments, a method described herein increases production of rAAV particles while maintaining or improving the quality attributes of the rAAV particles and compositions comprising thereof. In some embodiments, the quality of rAAV particles and compositions comprising thereof is assessed by determining the concentration of rAAV particles (e.g., GC/ml), the percentage of particles comprising a copy of the rAAV genome; the ratio of particles without a genome, infectivity of the rAAV particles, stability of rAAV particles, concentration of residual host cell proteins, or concentration of residual host cell nucleic acids (e.g., host cell genomic DNA, plasmid encoding rep and cap genes, plasmid encoding helper functions, plasmid encoding rAAV genome). In some embodiments, the quality of rAAV particles produced by a method described herein or compositions comprising thereof is the same as that of rAAV particles or compositions produced by a reference method using a host cell that does not comprise at least one modification that reduces or eliminates the activity of at least one endogenous gene or gene product in the apoptosis signaling pathway. In some embodiments, the quality of rAAV particles produced by a method described herein or compositions comprising thereof is better than the quality of rAAV particles or compositions produced by a reference method using a host cell that does not comprise at least one modification that reduces or eliminates the activity of at least one endogenous gene or gene product in the apoptosis signaling pathway.

Numerous cell culture-based systems are known in the art for production of rAAV particles, any of which can be used to practice a method described herein, rAAV production cultures for the production of rAAV virus particles require; (1) suitable host cells, including, for example, human-derived cell lines such as HeLa, A549, or HEK293 cells and their derivatives (HEK293T cells, HEK293F cells), or mammalian cell lines such as Vero, CHO cells or CHO-derived cells; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g., the helper may induce changes to the cellular environment that facilitate such AAV gene expression and replication. In some embodiments of a method described herein, AAV rep and cap genes, helper genes, and rAAV genomes are introduced into cells by transfection of one or more plasmid vectors encoding the AAV rep and cap genes, helper genes, and rAAV genome.

Molecular biology techniques to develop plasmid or viral vectors encoding the AAV rep and cap genes, helper genes, and/or rAAV genome are commonly known in the art. In some embodiments, AAV rep and cap genes are encoded by one plasmid vector. In some embodiments, AAV helper genes (e.g., adenovirus E1a gene, E1b gene, E4 gene, E2a gene, and VA gene) are encoded by one plasmid vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one plasmid vector. In some embodiments, one or more helper genes are stably expressed by the host cell, and one or more helper genes are introduced into the cell by transfection by one plasmid vector. In some embodiments, the helper genes are stably expressed by the host cell. In some embodiments, AAV rep and cap genes are encoded by one viral vector. In some embodiments, AAV helper genes (e.g., adenovirus E1a gene, E1b gene, E4 gene, E2a gene, and VA gene) are encoded by one viral vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one viral vector. In some embodiments, one or more helper genes are stably expressed by the host cell, and one or more helper genes are introduced into the cell by transfection by one viral vector. In some embodiments, the AAV rep and cap genes, the adenovirus helper functions necessary for packaging, and the rAAV genome to be packaged are introduced to the cells by transfection with one or more polynucleotides, e.g., vectors. In some embodiments, a method described herein comprises transfecting the cells with a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the AAV cap gene is an AAV8 or AAV9 cap gene. In some embodiments, the AAV cap gene is an AAV.rh8, AAV.rh10, AAV.rh20, AAV.rh39, AAV.Rh74, AAV.RHM4-1, AAV.hu32, AAV.hu37, AAV.PHB, or AAV.7m8 cap gene. In some embodiments, the AAV cap gene encodes a capsid protein with high sequence homology to AAV8 or AAV9 such as, AAV.rh10, AAV.rh20, AAV.rh39, AAV.Rh74, AAV.RHM4-1, AAV.hu32, and AAV.hu37. In some embodiments, the vector encoding the rAAV genome to be packaged comprises a gene of interest flanked by AAV ITRs. In some embodiments, the AAV ITRs are from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotype.

Any combination of vectors can be used to introduce AAV rep and cap genes, AAV helper genes, and rAAV genome to a cell in which rAAV particles are to be produced or packaged. In some embodiments of a method described herein, a first plasmid vector encoding an rAAV genome comprising a gene of interest flanked by AAV inverted terminal repeats (ITRs), a second vector encoding AAV rep and cap genes, and a third vector encoding helper genes can be used. In some embodiments, a mixture of the three vectors is co-transfected into a cell.

In some embodiments, a combination of transfection and infection is used by using both plasmid vectors as well as viral vectors.

In some embodiments, one or more of rep and cap genes, and AAV helper genes are constitutively expressed by the cells and does not need to be transfected or transduced into the cells. In some embodiments, the cell constitutively expresses rep and/or cap genes. In some embodiments, the cell constitutively expresses one or more AAV helper genes. In some embodiments, the cell constitutively expresses E1a. In some embodiments, the cell comprises a stable transgene encoding the rAAV genome.

In some embodiments, AAV rep, cap, and helper genes (e.g., E1a gene, E1b gene, E4 gene, E2a gene, or VA gene) can be of any AAV serotype. Similarly, AAV ITRs can also be of any AAV serotype. For example, in some embodiments, AAV ITRs are from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV cap gene is from AAV9 or AAV8 cap gene. In some embodiments, an AAV cap gene is from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV rep and cap genes for the production of a rAAV particle are from different serotypes. For example, the rep gene is from AAV2 whereas the cap gene is from AAV9.

Any suitable media known in the art can be used for the production of recombinant virus particles (e.g., rAAV particles) according to a method described herein. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety. In some embodiments, the medium comprises Dynamis™ Medium, FreeStyle™ 293 Expression Medium, or Expi293™ Expression Medium from Invitrogen/ThermoFisher. In some embodiments, the medium comprises Dynamis™ Medium. In some embodiments, a method described herein uses a cell culture comprising a serum-free medium, an animal-component free medium, or a chemically defined medium. In some embodiments, the medium is an animal-component free medium. In some embodiments, the medium comprises serum. In some embodiments, the medium comprises fetal bovine serum. In some embodiments, the medium is a glutamine-free medium. In some embodiments, the medium comprises glutamine. In some embodiments, the medium is supplemented with one or more of nutrients, salts, buffering agents, and additives (e.g., antifoam agent). In some embodiments, the medium is supplemented with glutamine. In some embodiments, the medium is supplemented with serum. In some embodiments, the medium is supplemented with fetal bovine serum. In some embodiments, the medium is supplemented with poloxamer, e.g., Kolliphor® P 188 Bio. In some embodiments, a medium is a base medium. In some embodiments, the medium is a feed medium.

Recombinant virus (e.g., rAAV) production cultures can routinely be grown under a variety of conditions (over a wide temperature range, for varying lengths of time, and the like) suitable to the particular host cell being utilized. As is known in the art, virus production cultures include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells. LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells and SF-9 cells which can be cultured in a variety of ways including, for example, spinner flasks, stirred tank bioreactors, and disposable systems such as the Wave bag system. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

Any cell or cell line that is known in the art to produce recombinant virus particles (e.g., rAAV particles) can be used in any one of the methods described herein. In some embodiments, a method of producing recombinant virus particles (e.g., rAAV particles) or increasing the production of recombinant virus particles (e.g., a rAAV particles) described herein uses HeLa cells, HEK293 cells, HEK293 derived cells (e.g., HEK293T cells, HEK293F cells), Vero cells, CHO cells, CHO-K1 cells, CHO derived cells, EB66 cells, LLC-MK cells, MDCK cells, RAF cells, RK cells, TCMK-1 cells, PK15 cells, BHK cells, BHK-21 cells, NS-1 cells, BHK cells, 293 cells, RK cells, Per.C6 cells, chicken embryo cells or SF-9 cells. In some embodiments, a method described herein uses mammalian cells. In some embodiments, a method described herein uses insect cells, e.g., SF-9 cells. In some embodiments, a method described herein uses cells adapted for growth in suspension culture. In some embodiments, a method described herein uses HEK293 cells adapted for growth in suspension culture.

In some embodiments, a cell culture described herein is a suspension culture. In some embodiments, a large-scale suspension cell culture described herein comprises HEK293 cells adapted for growth in suspension culture. In some embodiments, a cell culture described herein comprises a serum-free medium, an animal-component free medium, or a chemically defined medium. In some embodiments, a cell culture described herein comprises a serum-free medium. In some embodiments, suspension-adapted cells are cultured in a shaker flask, a spinner flask, a cell bag, or a bioreactor.

In some embodiments, a cell culture described herein comprises a serum-free medium, an animal-component free medium, or a chemically defined medium. In some embodiments, a cell culture described herein comprises a serum-free medium.

In some embodiments, a large-scale suspension culture cell culture described herein comprises a high-density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells. HEK293F cells), Vero cells, or SF-9 cells. In further embodiments, the cells are HEK293 cells.

Methods described herein can be used in the production of rAAV particles comprising a capsid protein from any AAV capsid serotype. In some embodiments, the rAAV particles comprise a capsid protein from an AAV capsid serotype selected from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B. AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a capsid protein that is a derivative, modification, or pseudotype of AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 capsid protein.

In some embodiments, the rAAV particles comprise a capsid protein from an AAV capsid serotype selected from AAV8 and AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein from an AAV capsid serotype selected from the group consisting of AAV.rh8, AAV.rh10, AAV.rh20, AAV.rh39, AAV.Rh74, AAV.RHM4-1, AAV.hu32, AAV.hu37, AAV.PHB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, AAV.hu32, and AAV.hu37.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%. 85%. 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1. VP2 and/or VP3 sequence of AAV.rh8, AAV.rh10, AAV.rh20, AAV.rh39, AAV.Rh74, AAV.RHM4-1, AAV.hu32, AAV.hu37, AAV.PHB, or AAV.7m8 capsid protein. In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, AAV.hu32, and AAV.hu37.

In additional embodiments, the rAAV particles comprise a mosaic capsid. In additional embodiments, the rAAV particles comprise a pseudotyped rAAV particle. In additional embodiments, the rAAV particles comprise a capsid containing a capsid protein chimera of two or more AAV capsid serotypes.

### RAAV Particles

The provided methods are suitable for use in the production of any isolated recombinant AAV particles. As such, the rAAV can be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7,AAV8, AAV9, AAV10, AAV1, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC1, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, rAAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10. AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%. 85%. 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAVPHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018. Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; U.S. Pat. Nos. 9,284,357; 9,409,953; 9,169,299; 9,193,956; 9458517; and 9,587,282; US patent application publication nos. 2015/0374803; 2015/0126588; 2017/0067908; 2013/0224836; 2016/0215024; 2017/0051257; and International Patent Application Nos. PCT/US2015/034799: PCT/EP2015/053335. In some embodiments, rAAV particles have a capsid protein at least 80% or more identical, e.g., 85%. 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; U.S. Pat. Nos. 9,284,357; 9,409,953; 9,169,299; 9,193.956; 9458517; and 9,587,282: US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2), WO 2005/033321 (see, e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6), WO 2006/110689, (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see. e.g., SEQ ID NOs: 80-294), and U.S. Appl. Publ. No. 20150023924 (see, e.g., SEQ ID NOs: 1, 5-10), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%. 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2). WO 2005/033321 (see. e.g., SEQ ID NOs: 123 and 88), WO 03/042397 (see, e.g., SEQ ID NOs: 2, 81, 85, and 97), WO 2006/068888 (see, e.g., SEQ ID NOs: 1 and 3-6). WO 2006/110689 (see, e.g., SEQ ID NOs: 5-38) WO2009/104964 (see, e.g., SEQ ID NOs: 1-5, 7, 9, 20, 22, 24 and 31), WO 2010/127097 (see, e.g., SEQ ID NOs: 5-38), and WO 2015/191508 (see, e.g., SEQ ID NOs: 80-294), and U.S. Appl. Publ. No. 20150023924 (see, e.g., SEQ ID NOs: 1, 5-10).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; U.S. Pat. Nos. 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257: International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for use in the production of recombinant AAV encoding a transgene. In certain embodiments, the transgene is from Tables 2A-2C. In some embodiments, the rAAV genome comprises a vector comprising the following components: (1) AAV inverted terminal repeats that flank an expression cassette; (2) regulatory control elements, such as a) promoter/enhancers, b) a polyA signal, and c) optionally an intron; and (3) nucleic acid sequences coding for a transgene. In other embodiments for expressing an intact or substantially intact monoclonal antibody (mAb), the rAAV genome comprises a vector comprising the following components: (1) AAV inverted terminal repeats that flank an expression cassette; (2) regulatory control elements, such as a) promoter/enhancers, b) a polyA signal, and c) optionally an intron; and (3) nucleic acid sequences coding for the light chain Fab and heavy chain Fab of the antibody, or at least the heavy chain or light chain Fab, and optionally a heavy chain Fe region. In still other embodiments for expressing an intact or substantially intact mAb, the rAAV genome comprises a vector comprising the following components: (1) AAV inverted terminal repeats that flank an expression cassette; (2) regulatory control elements, such as a) promoter/enhancers, b) a polyA signal, and c) optionally an intron; and (3) nucleic acid sequences coding for the heavy chain such as a Fab or full-length of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab, crovalimab, and eculizumab), anti-CD105 (e.g., carotuximab), anti-CC1Q (e.g., ANX-007), anti-TNFα (e.g., adalimumab, infliximab, and golimumab), anti-RGMa (e.g., clezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab); optionally an Fc polypeptide of the same isotvpe as the native form of the therapeutic antibody, such as an IgG isotype amino acid sequence IgG1, IgG2 or IgG4 or modified Fe thereof; and the light chain of an anti-VEGF (e.g., sevacizumab, ranibizumab, bevacizumab, and brolucizumab), anti-EpoR (e.g., LKA-651), anti-ALK1 (e.g., ascrinvacumab), anti-C5 (e.g., tesidolumab and eculizumab), anti-CD105 or anti-ENG (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab and sarilumab), anti-IL4R (e.g., dupilumab), anti-IL17A (e.g., ixekizumab and secukinumab), anti-IL-5 (e.g., mepolizumab), anti-IL12/IL23 (e.g., ustekinumnab), anti-CD19 (e.g., inebilizumab), anti-ITGF7 mAb (e.g., etrolizumab), anti-SOST mAb (e.g., romosozumab), anti-pKal mAb (e.g., lanadelumab), anti-ITGA4 (e.g., natalizumab), anti-ITGA4B7 (e.g., vedolizunab), anti-BLyS (e.g., belimumab), anti-PD-1 (e.g., nivolumab and pembrolizumab), anti-RANKL (e.g., densomab), anti-PCSK9 (e.g., alirocumab and evolocumab), anti-ANGPTL3 (e.g., evinacuNab), anti-OxPL (e.g., E06), anti-fD (e.g., lampalizumab), or anti-MMP9 (e.g., andecaliximab); wherein the heavy chain (such as a Fab and optionally Fe region) and the light chain are separated by a self-cleaving furin (F)/F2A or flexible linker, ensuring expression ofequal amounts of the heavy and the light chain polypeptides.

TABLE 2A

| Disease | Transgene |
|---|---|
| MPS I | alpha-L-iduronidase (IDUA) |
| MPS II (Hunter Syndrome) | iduronate-2-sulfatase (IDS) |
| ceroid lipofuscinosis (Batten disease) | (CLN1, CLN2, CLN10, CLN13), a soluble |
| | lysosomal protein (CLN5), a protein in the |
| | secretory pathway (CLN11), two cytoplasmic |
| | proteins that also peripherally associate with |
| | membranes (CLN4, CLN14), and many |
| | transmembrane proteins with different |
| | subcellular locations (CLN3, CLN6, CLN7, |
| | CLN8, CLN12) |
| MPS IIIa (Sanfilippo type A Syndrome) | heparan sulfate sulfatase (also called N- |
| | sulfoglucosamine sulfohydrolase (SGSH)) |
| MPS IIIB (Sanfilippo type B Syndrome) | N-acetyl-alpha-D-glucosaminidase (NAGLU) |
| MPS VI (Marotcaux-Lamy Syndrome) | arylsulfatasc B |
| Gaucher disease (type 1, II and III) | Glucocerebrosidase, GBA1 |
| Parkinson's Disease | Glucocerebrosidase; GBA1 |
| Parkinson's Disease | dopamine decarboxylase |
| Pompe | acid maltase; GAA |
| Metachromatic leukodystrophy | Aryl sulfatase A |
| MPS VII (Sly syndrome) | beta-glucuronidase |
| Disease | Transgene |
| MPS VIII | glucosamine-6-sulfate sulfatase |
| MPS IX | Hyaluronidase |
| Niemann-Pick disease | Sphingomyelinase |
| Niemann-Pick disease without | a npc 1 gene encoding a |
| sphingomyelinase deficiency | cholesterol metabolizing enzyme |
| Tay-Sachs disease | Alpha subunit of beta-hexosaminidase |
| Sandhoff disease | both alpha and beta subunit of beta- |
| | hexosaminidase |
| Fabry Disease | alpha-galactosidase |
| Fucosidosis | Fucosidase (FUCA1 gene) |
| Alpha-mannosidosis | alpha-mannosidase |
| Beta-mannosidosis | Beta-mannosidase |
| Wolman disease | cholesterol ester hydrolase |
| Parkinson's disease | Neurturin |
| Parkinson's disease | glial derived growth factor (GDGF) |
| Parkinson's disease | tyrosine hydroxylase |
| Parkinson's disease | glutamic acid decarboxylase. |
| Parkinson's disease | fibroblast growth factor-2 (FGF-2) |
| Parkinson's disease | brain derived growth factor (BDGF) |
| No disease listed (Galactosialidosis | neuraminidase deficiency with betagalactosidase |
| (Goldberg syndrome)) | deficiency |
| Spinal Muscular Atrophy (SMA) | SMN |
| Friedreich's ataxia | Frataxin |
| Amyotrophic lateral sclerosis (ALS) | SOD1 |
| Glycogen Storage Discase 1a | Glucose-6-phosphatase |
| XLMTM | MTM1 |
| Crigler Najjar | UGT1A1 |
| CPVT | CASQ2 |
| Rett syndrome | MECP2 |
| Achromatopsia | CNGB3, CNGA3, GNAT2, PDE6C |
| Choroidermia | CDM |
| Danon Disease | LAMP2 |
| Cystic Fibrosis | CFTR |
| Duchenne Muscular Dystrophy | Mini-Dystrophin or Microdystrophin Gene |
| Limb Girdle Muscular Dystrophy Type | human-alpha-sarcoglycan |
| 2C\|Gamma-sarcoglycanopathy | |
| Advanced Heart Failure | SERCA2a |
| Rheumatoid Arthritis | TNFR:Fc Fusion Gene |
| Leber Congenital Amaurosis | GAA |
| Limb Girdle Muscular Dystrophy Type | gamma-sarcoglycan |
| 2C\|Gamma-sarcoglycanopathy | |
| Retinitis Pigmentosa | hMERTK |
| Age-Related Macular Degeneration | SFLT01 |
| Becker Muscular Dystrophy and Sporadic | huFollistatin344 |
| Inclusion Body Myositis | |
| Parkinson's Disease | GDNF |
| Metachromatic Leukodystrophy (MLD) | cuARSA |
| Hepatitis C | anti-HCV shRNA |
| Limb Girdle Muscular Dystrophy Type 2D | hSGCA |
| Human Immunodeficiency Virus | PG9DP |
| Infections; HIV Infections (HIV-1) | |
| Acute Intermittant Porphyria | PBGD |
| Leber's Hereditary Optical Neuropathy | P1ND4v2 |
| Alpha-1 Antitrypsin Deficiency | alpha1AT |
| Pompe Disease | hGAA |
| X-linked Retinoschisis | RS1 |
| Choroideremia | hCHM |
| Giant Axonal Neuropathy | JeT-GAN |
| X-linked Retinoschisis | hRS1 |
| Squamous Cell Head and Neck Cancer; | hAQP1 |
| Radiation Induced Xerostomia | |
| Hemophilia B | Factor IX |
| Homozygous FH | hLDLR |
| Dysferlinopathies | dysferlin transgene (e.g., |
| | rAAVrh74.MHCK7.DYSF.DV) |
| Hemophilia B | AAV6 ZFP nuclease |
| MPS I | AAV6 ZFP nuclease |
| Rheumatoid Arthritis | NF-kB.IFN-β |
| Batten/CLN6 | CLN6 |
| Sanfilippo Disease Type A | hSGSH |
| Osteoarthritis | 5IL-1Ra |
| Achromatopsia | CNGA3 |
| Achromatopsia | CNGB3 |
| Ornithine Transcarbamylase (OTC) | OTC |
| Deficiency | |
| Hemophilia A | Factor VIII |
| Mucopolysaccharidosis II | ZFP nuclease |
| Hemophilia A | ZFP nuclease |
| Wet AMD | anti-VEGF |
| X-Linked Retinitis Pigmentosa | RPGR |
| Mucopolysaccharidosis Type VI | hARSB |
| Leber Hereditary Optic Neuropathy | ND4 |
| X-Linked Myotubular Myopathy | MTM1 |
| Crigler-Najjar Syndrome | UGT1A1 |
| Achromatopsia | CNGB3 |
| Retinitis Pigmentosa | hPDE6B |
| X-Linked Retinitis Pigmentosa | RPGR |
| Mucopolysaccharidosis Type 3 B | hNAGLU |
| Duchenne Muscular Dystrophy | GALGT2 |
| Arthritis, Rheumatoid; Arthritis, | TNFR:Fc Fusion Gene |
| Psoriatic; Ankylosing Spondylitis | |
| Idiopathic Parkinson's Disease | Neurturin |
| Alzheimer's Disease | NGF |
| Human Immunodeficiency Virus | tgAAC09 |
| Infections; HIV Infections (HIV-1) | |
| Familial Lipoprotein Lipase Deficiency | LPL |
| Idiopathic Parkinson's Disease | Neurturin |
| Alpha-1 Antitrypsin Deficiency | hAAT |
| Leber Congenital Amaurosis (LCA) 2 | hRPE65v2 |
| Batten Disease; Late Infantile Neuronal | CLN2 |
| Lipofuscinosis | |
| Parkinson's Disease | GAD |
| Sanfilippo Disease Type | N-sulfoglucosamine sulfohydrolase (SGSH) gene |
| A/Mucopolysaccharidosis Type IIIA | |
| Congestive Heart Failure | SERC2a |
| Becker Muscular Dystrophy and Sporadic | Follistatin (e.g., rAAV.CMV.huFollistatin344) |
| Inclusion Body Myositis | |
| Parkinson's Disease | hAADC-2 |
| Choroideremia | REP1 |
| CEA Specific AAV-DC-CTL Treatment in | CEA |
| Stage IV Gastric Cancer | |
| Gastric Cancer | MUC1-peptide-DC-CTL |
| Leber's Hereditary Optical Neuropathy | scAAV2-P1ND4v2 |
| Aromatic Amino Acid Decarboxylase | hAADC |
| Deficiency | |
| Hemophilia B | Factor IX |
| Parkinson's Disease | AADC |
| Leber Hereditary Optic Neuropathy | Genetic: GS010\|Drug: Placebo |
| SMA - Spinal Muscular Atrophy\|Gene | SMN |
| Therapy | |
| Hemophilia A | B-Domain Deleted Factor VIII |
| MPS I | IDUA |
| MPS II | IDS |
| CLN3-Related Neuronal Ceroid- | CLN3 |
| Lipofuscinosis (Batten) | |
| Limb-Girdle Muscular Dystrophy, Type | hSGCB |
| 2E | |
| Alzheimer Disease | APOE2 |
| Retinitis Pigmentosa | hMERKTK |
| Retinitis Pigmentosa | RLBP1 |
| Wet AMD or diabetic retinopathy | Anti-VEGF antibody or Anti-VEGF trap (e.g., |
| | one or more extracellular domains of VEGFR-1 |
| | and/or VEGFR-2; e.g., aflibercept) |

TABLE 2B

| | | ANTIBODIES | |
|---|---|---|---|
| ANTIGENS | | (TRANSGENE) | INDICATIONS |
| Nervous System | Amyloid beta | Solanezumab | Alzheimer's Disease |
| Targets | (Aβ or Abeta) | GSK933776 | |
| | peptides derived | | |
| | from APP | | |
| | Sortilin | AL-001 | Frontotemporal dementia |
| | | | (FTD) |
| | Tau protein | ABBV-8E12 | Alzheimer's, Progressive |
| | | UCB-0107 | supranuclear palsy, |
| | | NI-105 (BIIB076) | frontotemporal demential, |
| | | | chronic traumatic |
| | | | encephalopathy, Pick's |
| | | | complex, primary age- |
| | | | related taupathy |
| | Semaphorin-4D | VX15/2503 | Huntington's disease, |
| | (SEMA4D) | | juvenile Huntington's |
| | | | disease |
| | alpha-synuclein | Prasinezumab | Parkinson's disease, |
| | | NI-202 (BIIB054) | synucleinopathies |
| | | MED-1341 | |
| | superoxide | NI-204 | ALS, Alzheimer's Disease |
| | dismutase-1 | | |
| | (SOD-1) | | |
| | CGRP Receptor | eptinezumab, | Migraines, Cluster |
| | | fremanezumab | headaches |
| | | galcanezumab | |
| Ocular Anti- | VEGF | Sevacizumab | diabetic retinopathy (DR), |
| Angiogenic | | | myopic choroidal |
| Targets | | | neovascularization |
| | | | (mCNV), age-related |
| | | | macular degeneration |
| | | | (AMD), macular edema |
| | VEGF | ranibizumab | Wet AMD |
| | | (LUCENTIS ®) | |
| | | bevacizumab | |
| | | (AVASTIN ®) | |
| | | brolucizumab | |
| | erythropoietin | LKA-651 | retinal vein occlusion |
| | receptor | | (RVO), wet AMD, macular |
| | | | edema |
| | Amyloid beta | Solanezumab | Dry AMD |
| | (Aβ or Abeta) | GSK933776 | |
| | peptides derived | | |
| | from APP | | |
| | activin receptor | ascrinvacumab | neovascular age-related |
| | like kinase 1 | | macular degeneration |
| | (ALK1) | | |
| | complement | tesidolumab | dry AMD, uveitis |
| | component 5 | | |
| | (C5) | | |
| | endoglin (END | carotuximab | wet AMD and other retinal |
| | or CD105) | | disorders caused by |
| | | | increased vascularization |
| | complement | ANX-007 | glaucoma |
| | component 1Q | | |
| | (C1Q) | | |
| TNF-alpha | | adalimumab | uveitis |
| | | (HUMIRA ®) | |
| | | infliximab | |
| | | (REMICADE ®) | |
| | | golimumab | |
| Repulsive guidance molecule-A | | elezanumab | multiple sclerosis |
| Transthyretin (TTR) | | NI-301 | amyloidosis |
| | | PRX-004 | |
| Connective tissue growth factor (CTGF) | | pamrevlumab | fibrotic diseases, e.g., |
| | | | diabetic nephropathy, liver |
| | | | fibrosis, idiopathic |
| | | | pulmonary fibrosis |
| Neuromyelitis | interleukin | Satralizumab | NMO, DR, DME, uveitis |
| optica | receptor 6 | sarilumab | |
| (NMO)/Uveitis | (IL6R) | | |
| targets | CD19 | inebilizumab | NMO |

| Integrin beta 7 | etrolizumab | ulcerative colitis, Crohn's |
|---|---|---|
| | | disease |
| Sclerostin | romosozumab | Osteoporosis, abnormal |
| | (EVENITY ®) | bone loss or weakness |

TABLE 2C

| | | ANTIBODIES | |
|---|---|---|---|
| ANTIGENS | | (TRANSGENE) | INDICATIONS |
| Nervous System | Amyloid beta (Aβ | Aducanumab | Alzheimer's Disease |
| Targets | or Abeta) peptides | crenezumab | |
| | | gantenerumab | |
| | Tau protein | anti-TAU | Alzheimer's, Progressive |
| | | | supranuclear palsy, |
| | | | frontotemporal demential, |
| | | | chronic traumatic |
| | | | encephalopathy, Pick's |
| | | | complex, primary age- |
| | | | related taupathy |
| | CGRP Receptor | erenumab | Migraine |
| | | (AIMOVIG ™) | |
| Interleukins or | IL-17A | ixekizumab | Plaque psoriasis, psoriatic |
| interleukin | | (TALTZ ®) | arthritis, ankylosing |
| receptors | | secukinumab | sponylitis |
| | | (COSENTYX ®) | |
| | IL-5 | mepolizumab | Asthma |
| | | (NUCALA ®) | |
| | IL-12/IL-23 | ustekinumab | Psoriasis & Crohn's disease |
| | | (STELARA ®) | |
| | IL-4R | dupilumab | Atopic dermatitis |
| Integrin | | vedolizumab | Ulcerative colitis & |
| | | (ENTYVIO ®) | Crohn's disease |
| | | Natalizumab (anti- | Multiple sclerosis & |
| | | integrin alpha 4) | Crohn's disease |
| Cardiovascular | PCSK9 | alirocumab | HeFH & HoFH |
| Targets | | (PRALUENT ®) | |
| | | evolucomab | |
| | | (REPATHA ®) | |
| | ANGPTL3 | evinacumab | HoFH & severe forms of |
| | | | dyslipidema |
| | Proinflammatory/ | E06-scFv | Cardiovascular diseases |
| | proatherogenic | | such as atherosclerosis |
| | phospholipids | | |
| RANKL | | denosumab | Osteoporosis, increasing |
| | | (XGEVA ® and | bone mass in breast and |
| | | PROLIA ®') | prostate cancer patients, & |
| | | | preventing skeletal-related |
| | | | events due to bone |
| | | | metastasis |
| PD-1, or PD-L1 or PD-L2 | | nivolumab | Metastatic melanoma, |
| | | (OPDIVO ®) | lymphoma, non-small cell |
| | | pembrolizumab | lung carcinoma |
| | | (KEYTRUDA ®) | |
| BLyS (B-lymphocyte stimulator, also | | belimumab | Systemic lupus |
| known as B-cell activating factor (BAFF)) | | (BENLYSTA ®) | erythromatosis |
| Ocular Targets | Factor D | lampalizumab | Dry AMD |
| | MMP9 | andecaliximab | Dry AMD |
| TNF-alpha | | adalimumab | Rheumatoid arthritis, |
| | | (HUMIRA ®) and | psoriatic arthritis, |
| | | infliximab | askylosing spondylitis, |
| | | (REMICADE ®) | Crohn's disease, plaque |
| | | | psoriasis, ulcerative colitis |
| Plasma Protein | C5, C5a | eculizumab | Paroxysmal nocturnal |
| targets | | (SOLIRIS ® | hemoglobinuria, atypical |
| | | | hemolytic uremic |
| | | | syndrome, complement- |
| | | | mediated thrombotic |
| | | | microangiopathy |
| | Plasma kallikrein | lanadelumab | Hereditary angioedema |
| | | | (HAE) |

In some embodiments, the rAAV particles are rAAV viral vectors encoding an anti-VEGF antibody, such as a Fab. In specific embodiments, the rAAV particles are rAAV8-based viral vectors encoding an anti-VEGF antibody, such as a Fab. In more specific embodiments, the rAAV particles are rAAV8-based viral vectors encoding ranibizumab. In some embodiments, the rAAV particles are rAAV viral vectors encoding iduronidase (IDUA). In specific embodiments, the rAAV particles are rAAV9-based viral vectors encoding IDUA. In some embodiments, the rAAV particles are rAAV viral vectors encoding iduronate 2-sulfatase (IDS). In specific embodiments, the rAAV particles are rAAV9-based viral vectors encoding IDS. In some embodiments, the rAAV particles are rAAV viral vectors encoding a low-density lipoprotein receptor (LDLR). In specific embodiments, the rAAV particles are rAAV8-based viral vectors encoding LDLR. In some embodiments, the rAAV particles are rAAV viral vectors encoding tripeptidyl peptidase 1 (TPP1) protein. In specific embodiments, the rAAV particles are rAAV9-based viral vectors encoding TPP1. In some embodiments, the rAAV particles are rAAV viral vectors encoding non-membrane associated splice variant of VEGF receptor 1 (sFlt-1). In some embodiments, the rAAV particles are rAAV viral vectors encoding gamma-sarcoglycan, Rab Escort Protein 1 (REP1/CHM), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, microdystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), orithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3. AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al. 2001, Gene Therapy. Vol. 8, Number 16, Pages 1248-1254; and U.S. Pat. Nos. 6,596,535; 7,125,717; and 7,456,683, each of which is incorporated herein by reference in its entirety).

In some embodiments, the rAAV particles comprise a capsid protein from an AAV capsid serotype selected from AAV8 or AAV9. In some embodiments, the rAAV particles have an AAV capsid serotype of AAV8. In some embodiments, the rAAV particles have an AAV capsid serotype of AAV9.

In some embodiments, the rAAV particles comprise a capsid protein that is a derivative, modification, or pseudotype of AAV8 or AAV9 capsid protein. In some embodiments, the rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein.

In some embodiments, the rAAV particles comprise a capsid protein that is a derivative, modification, or pseudotype of AAV9 capsid protein. In some embodiments, the rAAV particles comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In additional embodiments, the rAAV particles comprise a mosaic capsid. Mosaic AAV particles are composed of a mixture of viral capsid proteins from different serotypes of AAV. In some embodiments, the rAAV particles comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise a mosaic capsid containing capsid proteins of a serotype selected from AAV1, AAV2, AAV5, AAV6, AAV7, AAV8, AAV9, AAV10, AAVrh.8, AAVrh.10, AAVhu.37, AAVrh.20, and AAVrh.74.

In additional embodiments, the rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16). In additional embodiments, the rAAV particles comprise a pseudotyped rAAV particle comprised of a capsid protein of an AAV serotype selected from AAV1, AAV2, AAV5, AAV6, AAV7, AAV8, AAV9, AAV10, AAVrh.8, and AAVrh.10, AAVhu.37, AAVrh.20, and AAVrh.74. In additional embodiments, the rAAV particles comprise a pseudotyped rAAV particle containing AAV8 capsid protein. In additional embodiments, the rAAV particles comprise a pseudotyped rAAV particle comprised of AAV9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, the rAAV particles comprise a capsid containing a capsid protein chimeric of two or more AAV capsid serotypes. In some embodiments, the rAAV particles comprise an AAV capsid protein chimeric of AAV8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV8 capsid protein and one or more AAV capsid proteins from an AAV serotype selected from AAV1, AAV2, AAV5, AAV6, AAV7, AAV9, AAV10, rAAVrh10, AAVrh.8, AAVrh.10, AAVhu.37, AAVrh.20, and AAVrh.74. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AA6, AAV7, AAV8, AAV9, AAVrh.8, AAVrh.10, AAVhu.37, AAVrh.20, and AAVrh.74.

Methods for Isolating rAAV Particles

In some embodiments, the disclosure provides methods for producing recombinant adeno-associated virus (rAAV) particles, comprising isolating rAAV particles from a feed comprising an impurity (for example, rAAV production culture). In some embodiments, a method for producing recombinant adeno-associated virus (rAAV) particles described herein comprises (a) isolating rAAV particles from a feed comprising an impurity (for example, rAAV production culture), and (b) formulating the isolated rAAV particles to produce the formulation.

In some embodiments, the disclosure further provides methods for producing a pharmaceutical unit dosage of a formulation comprising isolated recombinant adeno-associated virus (rAAV) particles, comprising isolating rAAV particles from a feed comprising an impurity (for example, rAAV production culture), and formulating the isolated rAAV particles.

Isolated rAAV particles can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, sterile filtration, or any combination(s) thereof. In some embodiments, downstream processing includes at least 2, at least 3, at least 4, at least 5 or at least 6 of: harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography. In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation. In some embodiments, the rAAV particles comprise a capsid protein of the AAV8 serotype. In some embodiments, the rAAV particles comprise a capsid protein of the AAV9 serotype.

In some embodiments, a method of isolating rAAV particles produced according to a method described herein comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles described herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles produced according to a method described herein comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles described herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, a method of isolating rAAV particles produced according to a method described herein comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles described herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In some embodiments, clarification of the harvested cell culture comprises sterile filtration. In some embodiments, the rAAV particles comprise a capsid protein of the AAV8 serotype. In some embodiments, the rAAV particles comprise a capsid protein of the AAV9 serotype.

Numerous methods are known in the art for production of rAAV particles, including transfection, stable cell line production, and infectious hybrid virus production systems which include adenovirus-AAV hybrids, herpesvirus-AAV hybrids and baculovirus-AAV hybrids, rAAV production cultures for the production of rAAV virus particles all require; (1) suitable host cells, including, for example, human-derived cell lines such as HeLa, A549, or HEK293 cells and their derivatives (HEK293T cells, HEK293F cells), mammalian cell lines such as Vero, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production. In some embodiments, the suitable helper virus function is provided by a recombinant polynucleotide described herein or a plasmid described herein. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors, rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells. BHK cells, 3T3 cells. 293 cells, RK cells, Per.C6 cells, chicken embryo cells or SF-9 cells which can be cultured in a variety of ways including, for example, spinner flasks, stirred tank bioreactors, and disposable systems such as the Wave bag system. In some embodiments, the cells are HEK293 cells. In some embodiments, the cells are HEK293 cells adapted for growth in suspension culture. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

In some embodiments, the rAAV production culture comprises a high-density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

In additional embodiments of the provided method the rAAV production culture comprises a suspension culture comprising rAAV particles. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety. In some embodiments, the suspension culture comprises a culture of mammalian cells or insect cells. In some embodiments, the suspension culture comprises a culture of HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells. LLC-MK cells, CV-1 cells, COS cells, MDBK cells, MDCK cells, CRFK cells. RAF cells, RK cells, TCMK-1 cells, LLCPK cells, PK15 cells. LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, the suspension culture comprises a culture of HEK293 cells.

In some embodiments, methods for the production of rAAV particles encompasses providing a cell culture comprising a cell capable of producing rAAV; adding to the cell culture a histone deacetylase (HDAC) inhibitor to a final concentration between about 0.1 mM and about 20 mM; and maintaining the cell culture under conditions that allows production of the rAAV particles. In some embodiments, the HDAC inhibitor comprises a short-chain fatty acid or salt thereof. In some embodiments, the HDAC inhibitor comprises butyrate (e.g., sodium butyrate), valproate (e.g., sodium valproate), propionate (e.g., sodium propionate), or a combination thereof.

In some embodiments, rAAV particles are produced as disclosed in WO 2020/033842, which is incorporated herein by reference in its entirety.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins, rAAV production cultures can further contain product-related impurities, for example, inactive vector forms, empty viral capsids, aggregated viral particles or capsids, mis-folded viral capsids, degraded viral particle.

In some embodiments, the rAAV production culture harvest is clarified to remove host cell debris. In some embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In some embodiments, clarification of the production culture harvest does not include centrifuigation.

In some embodiments, harvested cell culture is clarified using filtration. In some embodiments, clarification of the harvested cell culture comprises depth filtration. In some embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In some embodiments, the filter train comprises 2 depth filtration media and a sterile filtration media. In some embodiments, the depth filter media is a porous depth filter. In some embodiments, the filter train comprises Clarisolve® 20MS, Millistak+® COHC, and a sterilizing grade filter media. In some embodiments, the filter train comprises Clarisolve® 20MS, Millistak+® C0HC, and Sartopore®) 2 XLG 0.2 μm. In some embodiments, the harvested cell culture is pretreated before contacting it with the depth filter. In some embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the production culture harvest is clarified by filtration are disclosed in WO 2019/212921, which is incorporated herein by reference in its entirety.

In some embodiments, the rAAV production culture harvest is treated with a nuclease (e.g., Benzonase®) or endonuclease (e.g., endonuclease from Serratia marcescens) to digest high molecular weight DNA present in the production culture. The nuclease or endonuclease digestion can routinely be performed under standard conditions known in the art. For example, nuclease digestion is performed at a final concentration of 1-2.5 units/ml of Benzonase® at a temperature ranging from ambient to 37° C. for a period of 30 minutes to several hours.

Sterile filtration encompasses filtration using a sterilizing grade filter media. In some embodiments, the sterilizing grade filter media is a 0.2 or 0.22 μm pore filter. In some embodiments, the sterilizing grade filter media comprises polyethersulfone (PES). In some embodiments, the sterilizing grade filter media comprises polyvinylidene fluoride (PVDF). In some embodiments, the sterilizing grade filter media has a hydrophilic heterogeneous double layer design. In some embodiments, the sterilizing grade filter media has a hydrophilic heterogeneous double layer design of a 0.8 m pre-filter and 0.2 μm final filter membrane. In some embodiments, the sterilizing grade filter media has a hydrophilic heterogeneous double layer design of a 1.2 μm pre-filter and 0.2 μm final filter membrane. In some embodiments, the sterilizing grade filter media is a 0.2 or 0.22 μm pore filter. In further embodiments, the sterilizing grade filter media is a 0.2 μm pore filter. In some embodiments, the sterilizing grade filter media is a Sartopore® 2 XLG 0.2 m, Durapore™ PVDF Membranes 0.45 μm, or Sartoguard® PES 1.2 μm+0.2 μm nominal pore size combination. In some embodiments, the sterilizing grade filter media is a Sartopore® RG 2 XLG 0.2 μm.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul et al., Human Gene Therapy 4:609-615 (1993). TFF concentration of the clarified feed enables a technically manageable volume of clarified feed to be subjected to chromatography and allows for more reasonable sizing of columns without the need for lengthy recirculation times. In some embodiments, the clarified feed is concentrated between at least two-fold and at least ten-fold. In some embodiments, the clarified feed is concentrated between at least ten-fold and at least twenty-fold. In some embodiments, the clarified feed is concentrated between at least twenty-fold and at least fifty-fold. In some embodiments, the clarified feed is concentrated about twenty-fold. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In some embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed described herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™ POROS™ CaptureSelect™ AAVX affinity resin, POROS™ CaptureSelect™ AAV9 affinity resin, and POROS™ CaptureSelect™ AAV8 affinity resin. In some embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, UNOspherc™ Q (Biorad, Hercules. Calif.), and N-charged amino or imino resins such as e.g., POROS™ 50 PI, or any DEAE, TMAE, tertiary or quaternary amine, or PEI-based resins known in the art (U.S. Pat. No. 6,989,264; Brument et al., Mol. Therapy 6(5):678-686 (2002); Gao et al., Hum. Gene Therapy 11:2079-2091 (2000)). In some embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers. In some embodiments, the monolith anion exchange chromatography media is selected from the group consisting of CIMmultus™ QA-1 Advanced Composite Column (Quaternary amine), CIMmultus™ DEAE-1 Advanced Composite Column (Diethylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In some embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In some embodiments, anion exchange chromatography is performed according to a method disclosed in WO 2019/241535, which is incorporated herein by reference in its entirety.

In some embodiments, a method of isolating rAAV particles comprises determining the vector genome titer, capsid titer, and/or the ratio of full to empty capsids in a composition comprising the isolated rAAV particles. In some embodiments, the vector genome titer is determined by quantitative PCR (qPCR) or digital PCR (dPCR) or droplet digital PCR (ddPCR). In some embodiments, the capsid titer is determined by serotype-specific ELISA. In some embodiments, the ratio of fill to empty capsids is determined by Analytical Ultracentrifugation (AUC) or Transmission Electron Microscopy (TEM).

In some embodiments, the vector genome titer, capsid titer, and/or the ratio of full to empty capsids is determined by spectrophotometry, for example, by measuring the absorbance of the composition at 260 nm; and measuring the absorbance of the composition at 280 nm. In some embodiments, the rAAV particles are not denatured prior to measuring the absorbance of the composition. In some embodiments, the rAAV particles are denatured prior to measuring the absorbance of the composition. In some embodiments, the absorbance of the composition at 260 nm and 280 nm is determined using a spectrophotometer. In some embodiments, the absorbance of the composition at 260 nm and 280 nm is determined using an HPLC. In some embodiments, the absorbance is peak absorbance. Several methods for measuring the absorbance of a composition at 260 nm and 280 nm are known in the art. Methods of determining vector genome titer and capsid titer of a composition comprising the isolated recombinant rAAV particles are disclosed in WO 2019/212922, which is incorporated herein by reference in its entirety.

In additional embodiments the disclosure provides compositions comprising isolated rAAV particles produced according to a method described herein. In some embodiment, the composition is a pharmaceutical composition comprising a pharmaceutically acceptable carrier.

As used herein the term “pharmaceutically acceptable” means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. Pharmaceutical compositions can be formulated to be compatible with a particular route of administration or delivery, as set forth herein or known to one of skill in the art. Thus, pharmaceutical compositions include carriers, diluents, or excipients suitable for administration by various routes. Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993). Technonic Publishing Co., Inc., Lancaster, Pa.; Ansel and Stoklosa, Pharmaceutical Calculations (2001) 11th ed., Lippincott Williams & Wilkins, Baltimore, Md.; and Poznanskv et al., Drug Delivery Systems (1980). R. L. Juliano, ed., Oxford, N.Y., pp. 253-315).

In some embodiments, the composition is a pharmaceutical unit dose. A “unit dose” refers to a physically discrete unit suited as a unitary dosage for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dose forms may be within, for example, ampules and vials, which may include a liquid composition, or a composition in a freeze-dried or lyophilized state: a sterile liquid carrier, for example, can be added prior to administration or delivery in vivo. Individual unit dose forms can be included in multi-dose kits or containers. Recombinant vector (e.g., AAV) sequences, plasmids, vector genomes, and recombinant virus particles, and pharmaceutical compositions thereof can be packaged in single or multiple unit dose form for ease of administration and uniformity of dosage.

In some embodiments, the composition comprises rAAV particles comprising an AAV capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the AAV capsid serotype is AAV8. In some embodiments, the AAV capsid serotype is AAV9.

### Methods of Producing a Recombinant Polypeptide

In one aspect, the disclosure provides a method of expressing a recombinant polypeptide in a eukaryotic host cell described herein. In some embodiments, the method further comprises recovering the polypeptide.

In some embodiments, the disclosure provides a method of producing a recombinant polypeptide, comprising (a) providing a cell culture comprising a plurality of cells described herein comprising one or more polynucleotides encoding the recombinant polypeptide; and (b) maintaining the cell culture under conditions that allow production of the recombinant polypeptide. In some embodiment, the recombinant polypeptide is an antibody or antibody fragment. In some embodiments, the antibody is a chimeric, human or humanized antibody. In some embodiments, the antibody is an IgG antibody. In some embodiments, the recombinant polypeptide is a fusion protein. In some embodiments, the fusion protein comprises an antibody or fragment thereof. In some embodiments, the fusion protein comprises an Fc region. In some embodiments, the fusion protein comprises an antigen-binding antibody fragment. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell. In some embodiments, the cell culture is a suspension culture or an adherent culture. In some embodiments, the cell culture is a suspension culture. In some embodiments, the cell culture has a volume between about 50 liters and about 20,000 liters.

In some embodiments, the disclosure provides a method for producing rAAV particles, comprising culturing a cell described herein, wherein the cell comprises one or more polynucleotides encoding the recombinant polypeptide. In some embodiment, the recombinant polypeptide is an antibody or antibody fragment. In some embodiments, the antibody is a chimeric, human or humanized antibody. In some embodiments, the antibody is an IgG antibody. In some embodiments, the recombinant polypeptide is a fusion protein. In some embodiments, the fusion protein comprises an antibody or fragment thereof. In some embodiments, the fusion protein comprises an Fc region. In some embodiments, the fusion protein comprises an antigen-binding antibody fragment. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell.

In some embodiments, the disclosure provides a method of producing rAAV particles, comprising (a) providing a cell culture comprising a plurality of cells; (b) introducing into the cells one or more polynucleotides encoding the recombinant polypeptide; and (c) maintaining the cell culture under conditions that allow production of the encoding the recombinant polypeptide. In some embodiment, the recombinant polypeptide is an antibody or antibody fragment. In some embodiments, the antibody is a chimeric, human or humanized antibody. In some embodiments, the antibody is an IgG antibody. In some embodiments, the recombinant polypeptide is a fusion protein. In some embodiments, the fusion protein comprises an antibody or fragment thereof. In some embodiments, the fusion protein comprises an Fc region. In some embodiments, the fusion protein comprises an antigen-binding antibody fragment. In some embodiments, the cell is a HEK293 cell. In some embodiments, the cell is a HEK293 derived cell. In some embodiments, the cell is a suspension adapted HEK293 cell. In some embodiments, the cell is a suspension adapted HEK293 derived cell. In some embodiments, the cell culture is a suspension culture or an adherent culture. In some embodiments, the cell culture is a suspension culture. In some embodiments, the cell culture has a volume between about 50 liters and about 20,000 liters.

### EXAMPLES

### Example 1. Identification of Target Genes for Modification

As disclosed in Intl. Appl. Publ. No. WO 2023/24392, which is incorporated herein by reference in its entirety, the addition of a caspase inhibitor to a recombinant AAV producing cell culture increases the rAAV yield. To identify candidate genes for modification in a host cell to improve AAV productivity, a screen using an arrayed RNAi library targeting apoptosis signaling pathway genes was implemented.

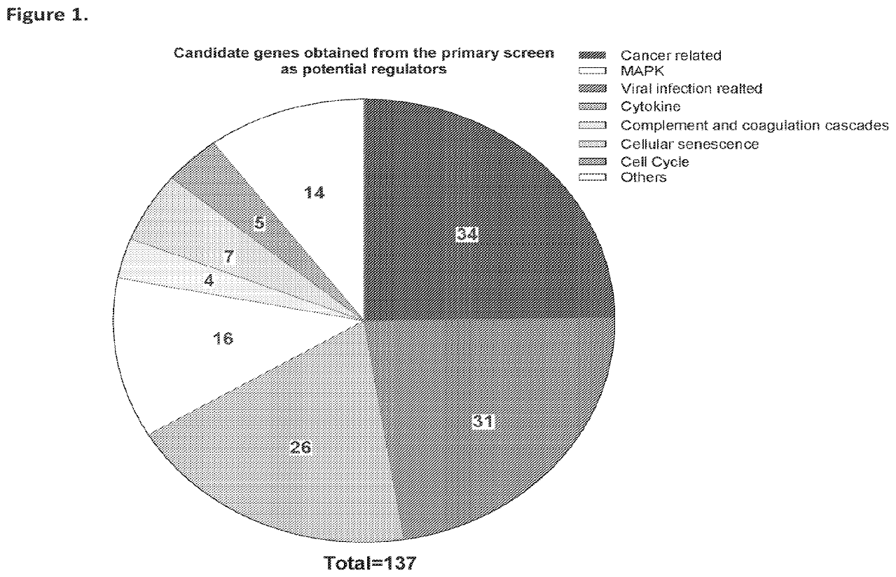

To systematically understand the genetic profiles of an HEK293 cell line and cellular factors that modulate AAV production, a high-throughput RNAi screen using a library targeting the apoptosis signaling pathway was performed. The initial screen assessed the contribution of 923 human genes using 2769, i.e., 3 per gene, unique siRNAs form the Invitrogen Silencer™ Library. Briefly, on day 1, 96-well plates HEK293 cells were seeded in collagen-I pre-coated 24-well plates at ~40~50% confluency and reverse transfected with a single siRNA capable of downregulating the mRNA expressed by a specific target gene. Approximately 24 hrs later, the cells were transfected with a combination of three plasmids (i.e., helper, cis and trans plasmids) to produce rAAV particles. On day 4, cell suspension from each well were lysed into AAV-MAX™ lysis buffer and the titer of rAAV particles produced was determined using ddPCR. The screen used the Helper #5 helper plasmid (see, WO 2023/060113) and the cis and trans plasmids encoding the AAV8 TG-A recombinant AAV particle. The initial screen produced evaluable results with biological repeats for 845 target genes, based on which 267 genes were selected for further validation. Similar to the initial screen, the validation screen used transfection mediated RNAi to downregulate mRNA produced by the target genes in adherent HEK293 cells, followed by transfection with the combination of three plasmids (i.e., Helper #5 helper, cis and trans plasmids) to produce rAAV particles. The titer of the AAV8 TG-A, AAV9 TG-D and AAV8 TG-B recombinant AAV particles was independently measured in the validation screen. This unbiased screening approach identified 137 genes whose downregulation increased recombinant AAV production as reflected by an increase in titer. Gene-ontology analysis of these candidate genes affecting titers reveals functional categorization related to several signaling pathways. FIG. 1. More than half of the genes identified have been linked to pathways involved in the immune reaction to viruses.

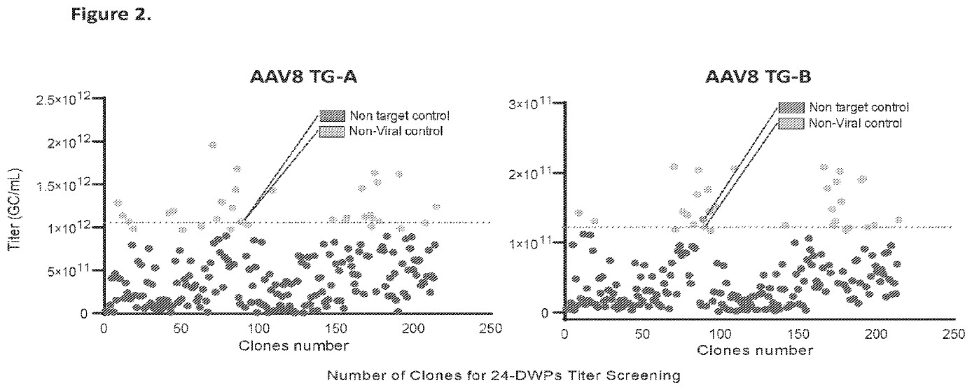

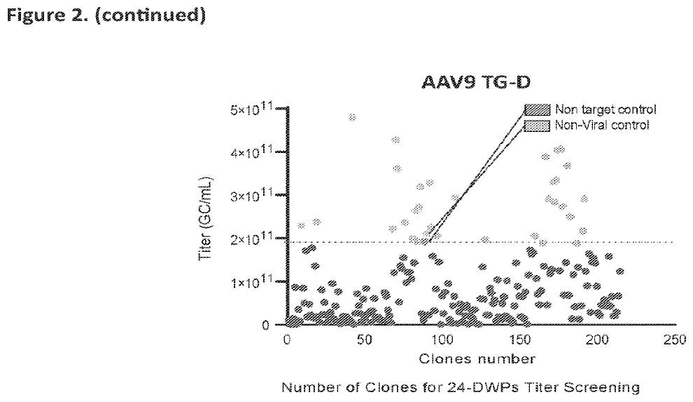

To further confirm the results from the primary screening and to identify siRNAs targeting genes with a specific effect on titer improvement without significantly affecting cell viability during long-term cell culture, a lentivirus-based shRNA assay was established. Lentiviral delivery of RNAi vectors provided the advantages of permanent integration of the RNAi vector into the host cell genome, high transduction efficiency and relatively low cost, which made this approach suitable for further testing in suspension cultures. Two individual lentiviral shRNA vectors were produced for each of the 137 genes identified by the primary screening. The secondary testing was performed in 24-well deep well plates using HEK293 suspension cultures, rAAV particles were produced by triple transfection using the Helper #5 helper plasmid (see, WO 2023/060113). Titers were independently measured for the AAV8 TG-A. AAV8 TG-B and AAV9 TG-D recombinant AAV particles. FIG. 2, rAAV titers were determined with ddPCR as GC/ml of suspension culture. In all instances, there was no significant difference in rAAV titers between cells infected with the scrambled shRNA control vector and control cells without lentivirus infection, indicating that host cells fully recovered after lentiviral infection and scaling up. The 19 genes whose downregulation produced the highest rAAV titers are listed in the Table below. Most of the genes (15 out of 19) after shRNA mediated knock-down showed similar % titer improvement for all three different rAAV particles tested. However, VEGFB knock-down produced significantly more improvement in AAV8 TG-B and AAV9 TG-D titers; FIS1 knock-down increased AAV8 TG-B and AAV9 TG-D titers but lowered AAV8 TG-A titer; BNIP1 knock-down significantly improved AAV8 TG-A titer only, and CUL1 knock-down significantly improved AAV8 TG-B and AAV9 TG-D titers but lowered AAV8 TG-A titer. The data is consistent with an understanding that most genes listed in the Table had universal effect on titer improvement, while some genes affected the titers indirectly through other signaling pathways which may be transgene-specific.

TABLE 3
Absolute and relative rAAV titers following lentivirus-based shRNA silencing
of target genes in 24-well deep well suspension cultures. Absolute titer is
GC/ml of suspension culture. Relative titer of non-target control is 1.

| | AAV8 TG-A titer | | AAV8 TG-B titer | | AAV9 TG-D titer | |
|---|---|---|---|---|---|---|
| Target gene | absolute | relative | absolute | relative | absolute | relative |
| Non-target control | 1.0 × 10¹² | 1.0 | 1.2 × 10¹¹ | 1.0 | 1.9 × 10¹¹ | 1.0 |
| ANGPT1 | 1.3 × 10¹² | 1.3 | 1.2 × 10¹¹ | 1.0 | 2.5 × 10¹¹ | 1.3 |
| ARHGEF7 | 1.2 × 10¹² | 1.2 | 1.8 × 10¹¹ | 1.5 | 2.9 × 10¹¹ | 1.5 |
| ARHGEF17 | 1.4 × 10¹² | 1.4 | 2.1 × 10¹¹ | 1.8 | 2.9 × 10¹¹ | 1.5 |
| BID | 1.2 × 10¹² | 1.2 | 1.3 × 10¹¹ | 1.1 | 2.0 × 10¹¹ | 1.1 |
| BNIP1 | 1.9 × 10¹² | 1.9 | 1.3 × 10¹¹ | 1.1 | 2.2 × 10¹¹ | 1.2 |
| C19orf2 | 1.6 × 10¹² | 1.6 | 1.9 × 10¹¹ | 1.6 | 4.0 × 10¹¹ | 2.1 |
| CHST11 | 1.6 × 10¹² | 1.6 | 1.9 × 10¹¹ | 1.6 | 2.9 × 10¹¹ | 1.5 |
| CUL1 | 0.7 × 10¹² | 0.7 | 1.2 × 10¹¹ | 1.0 | 3.7 × 10¹¹ | 1.9 |
| DAP3 | 1.5 × 10¹² | 1.5 | 2.1 × 10¹¹ | 1.8 | 3.9 × 10¹¹ | 2.1 |
| DYNLL1 | 1.5 × 10¹² | 1.5 | 2.0 × 10¹¹ | 1.7 | 4.1 × 10¹¹ | 2.2 |
| FIS1 | | | 1.8 × 10¹¹ | 1.5 | 3.3 × 10¹¹ | 1.7 |
| HMGB2 | 1.3 × 10¹² | 1.3 | 1.4 × 10¹¹ | 1.2 | 2.4 × 10¹¹ | 1.3 |
| IGFBP3 | 2.0 × 10¹² | 2.0 | 2.1 × 10¹¹ | 1.8 | 4.3 × 10¹¹ | 2.3 |
| NRG1 | 1.2 × 10¹² | 1.2 | 1.3 × 10¹¹ | 1.1 | 1.6 × 10¹¹ | 0.8 |
| TNFRSF10C | 1.3 × 10¹² | 1.3 | 1.5 × 10¹¹ | 1.3 | 3.3 × 10¹¹ | 1.7 |
| TNFSF12 | 1.2 × 10¹² | 1.2 | 1.3 × 10¹¹ | 1.1 | 3.4 × 10¹¹ | 1.8 |
| MAL | 1.2 × 10¹² | 1.2 | 1.3 × 10¹¹ | 1.1 | 2.6 × 10¹¹ | 1.4 |
| MAX | 1.7 × 10¹² | 1.7 | 2.0 × 10¹¹ | 1.7 | 3.2 × 10¹¹ | 1.7 |
| VEGFB | 1.2 × 10¹² | 1.2 | 3.9 × 10¹¹ | 3.3 | 4.8 × 10¹¹ | 2.5 |

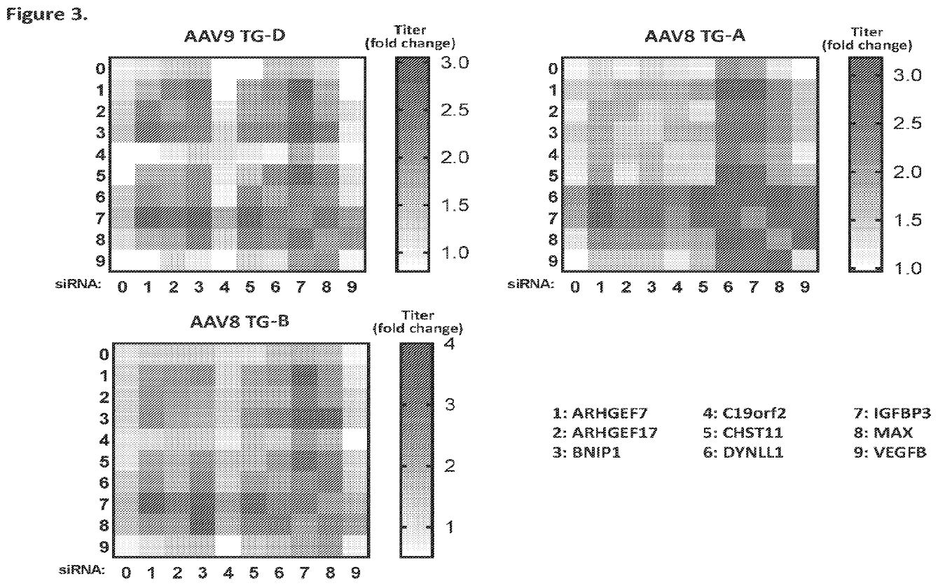

The effect of simultaneously lowering the activity of 2 target genes on rAAV titer was determined. Briefly, on day 1. HEK293 cells were seeded in collagen-I pre-coated 24-well plates at ~40-50% confluency and reverse co-transfected with specific siRNAs targeting 2 of the top 9 candidate genes using the same amount of siRNA for each gene. Approximately 24 hrs later, the cells were transfected with a combination of three plasmids (i.e., helper, cis and trans plasmids) to produce rAAV particles. On day 4, cell suspension from each well were lysed into AAV-MAX™ lysis buffer and the titer of rAAV particles produced was determined using ddPCR. The screen used the Helper #5 helper plasmid (see, WO 2023/060113) and the cis and trans plasmids encoding the AAV8 TG-A, AAV8 TG-B or AAV9 TG-D recombinant AAV particles. Results are shown in FIG. 3.

### Example 2. Host Cell Engineering Through Knocking-Out IGFBP3

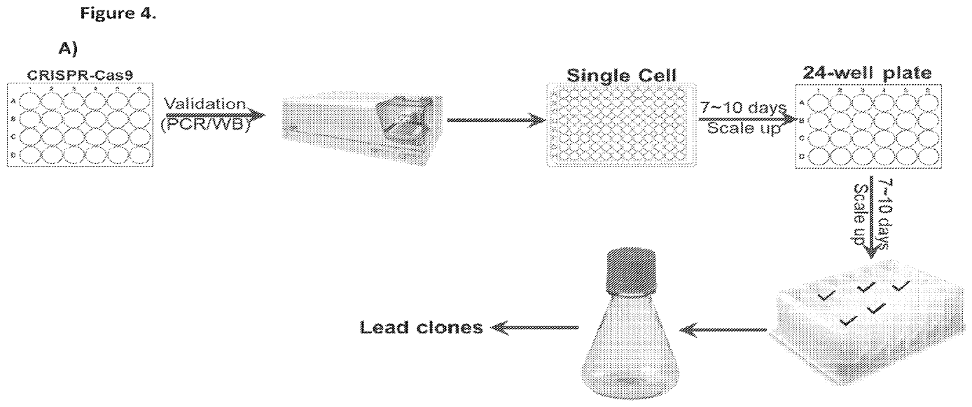

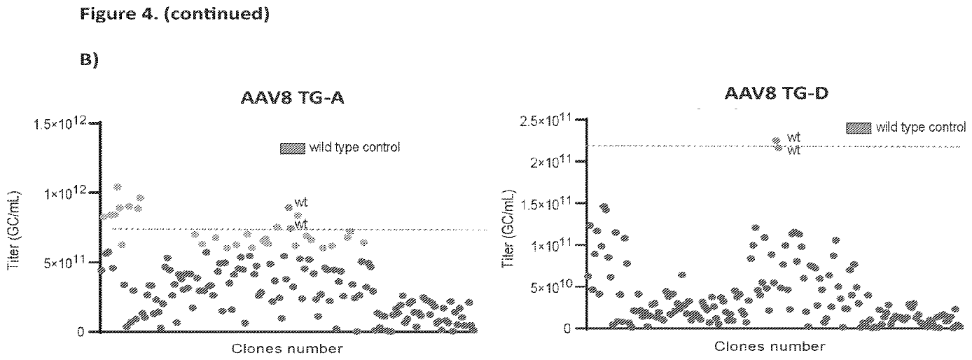

Genes with the most pronounced effect on rAAV titers, such as IGFBP3, were selected for CRISPR/Cas9 mediated knockout editing. FIG. 4A. Briefly, on day 1, HEK293 cells were be seeded in collagen-I pre-coated 24-well plates at ~40~50% confluency and reverse co-transfected with Cas9 protein and specific sgRNA targeting the IGFBP3 gene. Approximately 48 hrs later, two wells of cells were harvested for evaluating editing efficiency by PCR and Western blot. On day 4, the rest of cells were trypsinized and resuspended for single cell cloning into 96-well plates. On about day 14 to 18, the emerging clones were transferred into 24-well plates. 7-10 days later the surviving clones were transferred to 24-deep well plates to generate sufficient cells for determining IGFPBP3 expression by Western blot and for measuring AAV production. AAV production was measured by transfecting the cells with a combination of three plasmids, i.e., helper, transgene coding cis and rep/cap coding trans plasmids, to produce AAV8 TG-A, AAV8 TG-B or AAV9 TG-D rAAV particles. 72 hours post triple transfection, cell suspension of each clone were lysed into AAV-MAX™ lysis buffer and titer of AAV particles produced were determined using ddPCR.

Following transfection with Cas9 protein and IGFBP3 specific sgRNA, 40.8% (170 out of 416) clones in 96-well were successfully expanded to 24-well. This is only about half of the cloning efficiency after treatment with random control sgRNA (>80%). 94.1% (160 out of 170) clones in 24-well survived after being transferred into 24-well deep-well plates. FIG. 4B shows the AAV titers produced by the candidate clones following transfection with the helper #5/TG-A/AAV8 or helper #5/TG-D/AAV9 helper/cis/trans plasmid combinations. Most of the candidate clones produced the same or lower titer than the control cells.

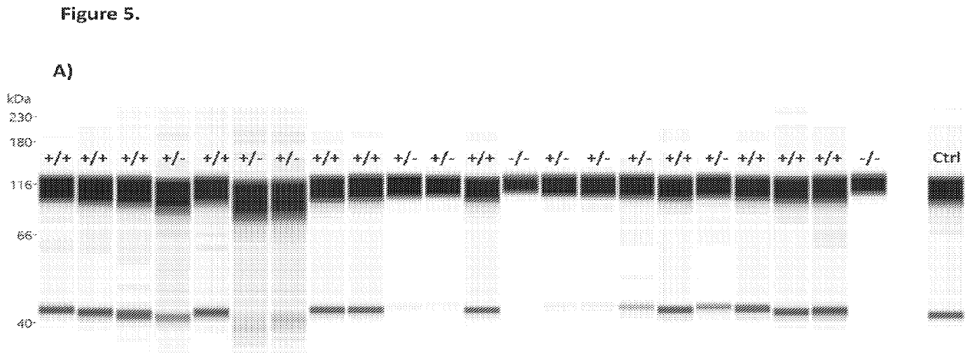

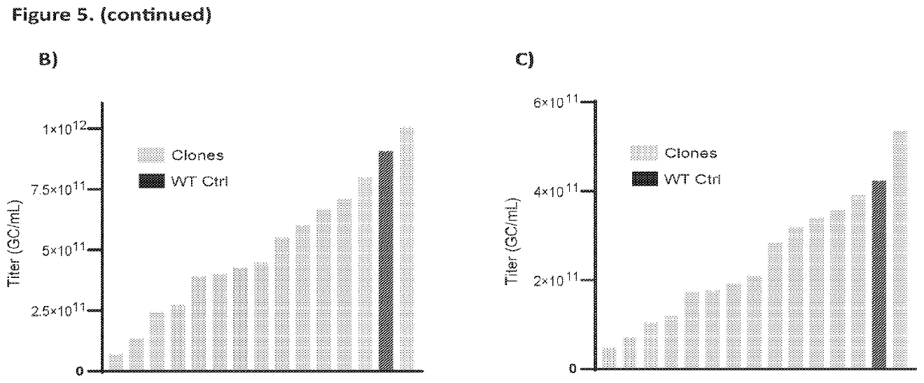

As shown in FIG. 5A, the most candidate clones were assessed to have a +/+ wild-type or +/− heterozygous mutant IGFBP3 genotype based on IGFBP3 expression detected by Western blotting. Further testing failed to identify a clone capable of producing significantly higher AAV yield than the control cells. FIGS. 5B and C.

### Example 3. Host Cell Engineering Through Knocking-Out DYNLL1

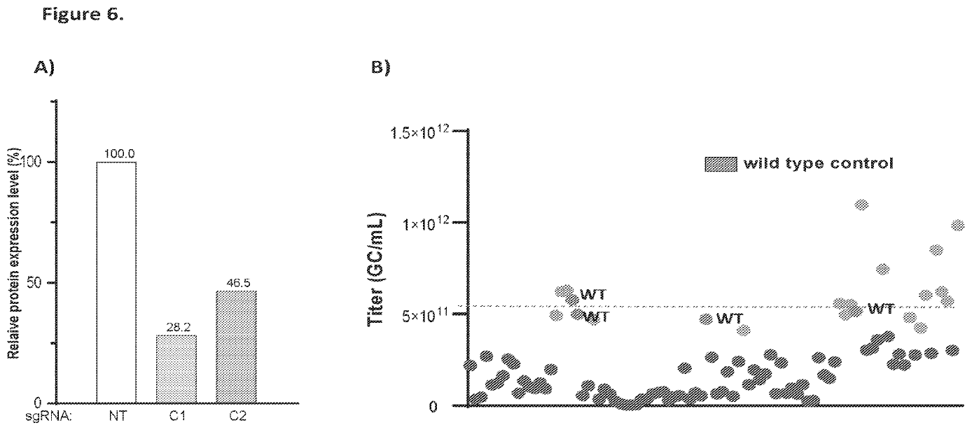

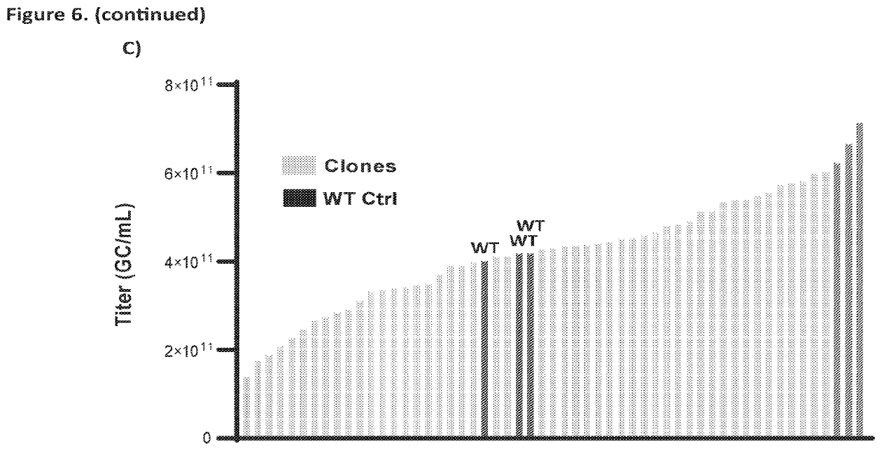

Candidate DYNLL1-KO clones were generated substantially as described above. The efficiency of two DYNLL1 specific sgRNAs (C1 and C2) were tested by a Western blotting on cells co-transfected with Cas9 protein and a DYNLL1 specific sgRNA. FIG. 6A. 49% (357 out of 744) clones in 96-well were successfully expanded to 24-well, which is still about half of the cloning efficiency following treatment with random control sgRNA (>80%). AAV production of candidate clones was measured following transfection with the helper #5/TG-A/AAV8 (FIG. 6B) and helper #5/TG-D/AAV9 (FIG. 6C) helper/cis/trans plasmid combinations. A significant portion of the candidate clones produced a higher AAV titer than the parental control clone. Clones with similar or better titers than the parental control clone were selected as potential candidate clones for further evaluation. DYNLL1 genotype of candidate clones was assessed by sequencing and ICE analysis. DYNLL1+/− heterozygote clones produced higher AAV titers than DYNLL1+/+WT or DYNLL1 −/− KO clones. DYNLL1 −/− KO clones had significantly slower growth rate compared to wild type and heterozygotes clones. Without being bound by any specific theory, the slower growth rate of −/− KO clones may have been the reason for these clones not displaying a significant improvement in AAV production. DYNLL1 −/− KO clones exhibit a significantly slower growth rate compared to the DYNLL1+/− heterozygote (28-30 hours vs 22-24 hours for doubling time). Despite the slower doubling time, AAV production by the DYNLL1 −/− KO clones remained comparable to that of the DYNLL1+/− heterozygote, with the DYNLL1 −/− KO clone achieving about 80-90% of the titers achieved by the DYNLL1+/− heterozygote. Previous experiments (data not shown) compared the effect of viable cell density (VCD) on AAV production and found that a culture with VCD of 6.5E6 during transfection produced a 30~40% higher AAV titer than a culture with 5E6 VCD during transfection. A culture of DYNLL1 −/− KO clone cells with ~29 hours doubling time may not have reached a VCD of 6.5e6 during transfection if it was seeded at the same density as a culture comprising corresponding DYNLL1 +/− or DYNLL1+/+ cells. Therefore, the slower doubling time may explain why the DYNLL1 −/− clone produced lower AAV titers than a DYNLL1+/− clone.

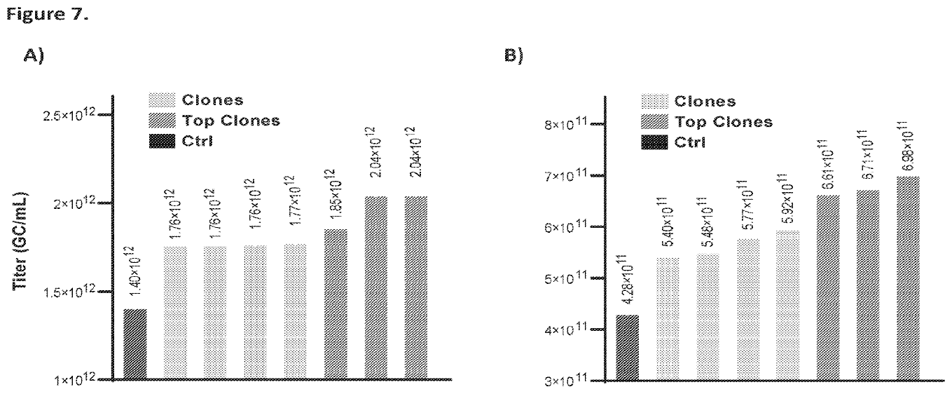

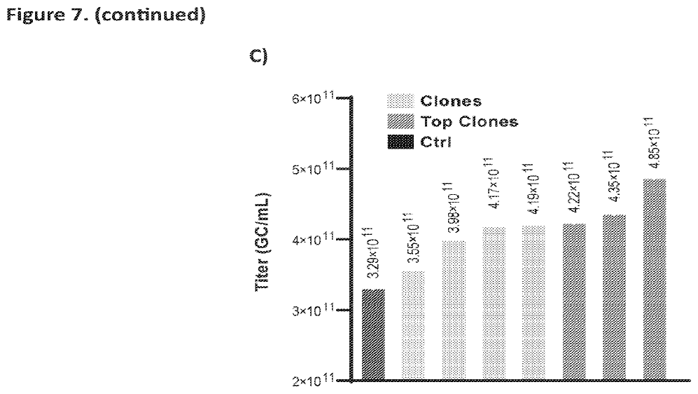

FIG. 7 shows the AAV titer produced by the 1C1, 4D9, 2C6, 4A8, 3B7, 4D5, and 4A2 DYNLL1+/− clones following transfection with (i) pHRC #7 (helper+AAV8-trans) plasmid and TG-A cis plasmid (ii) the helper #5, TG-D cis and AAV9 trans plasmids, and (iii) the pHRC #7 (helper+AAV8-trans) plasmid and TG-B cis plasmid. The pHRC #7 helper/rep/cap plasmid is disclosed in PCT/US2024/023368, filed Apr. 5, 2024. The titer increased similarly for all three transgenes tested in the 1C1, 4D9, 2C6, 4A8, 3B7, 4D5, and 4A2 clones.

35% of the clones selected following transfection with Cas-9/sgRNA-C1 were identified as DYNLL1 −/−, whereas only 1% of the clones identified after transfection with Cas-9/sgRNA-C1 clones were identified as DYNLL1 −/−.

### Example 4. Host Cell Engineering Through Knocking-Out DYNLL1 and MAX

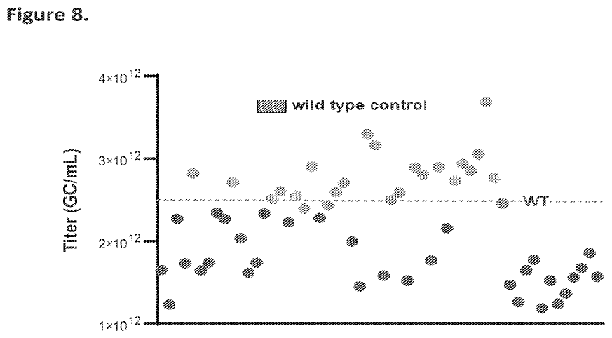

Candidate DYNLL1/MAX double KO clones were generated substantially as described above by using the 4A2 and 4D5 DYNLL1+/− cells as the starting point. 4A2 or 4D5 cells were transfected with Cas-9 protein and a MAX specific sgRNA. The MAX sgRNA KO efficiency was assessed by sequencing on sample collected 1 day after transfection. 65.8% (252 out of 383) 4A2 derived clones in 96-well were successfully expanded to 24-well, while only 4.2% (16 out of 384) of 4D5 derived clones in 96-well were successfully expanded to 24-well. AAV production of candidate clones was measured following transfection with the pHRC #7 (helper+AAV8-trans) plasmid and TG-A cis plasmid. FIG. 8. A significant portion of the candidate clones produced a higher AAV titer than the parental control clone. Clones with similar or better titers than the parental control clone were selected as potential candidate clones for further evaluation.

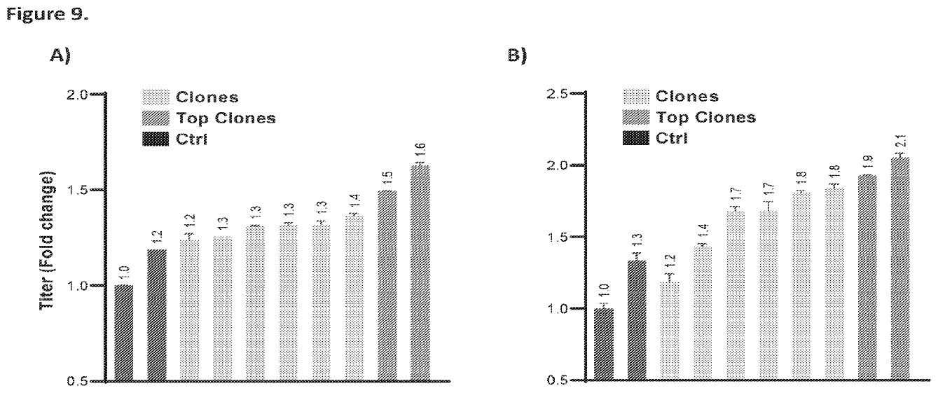

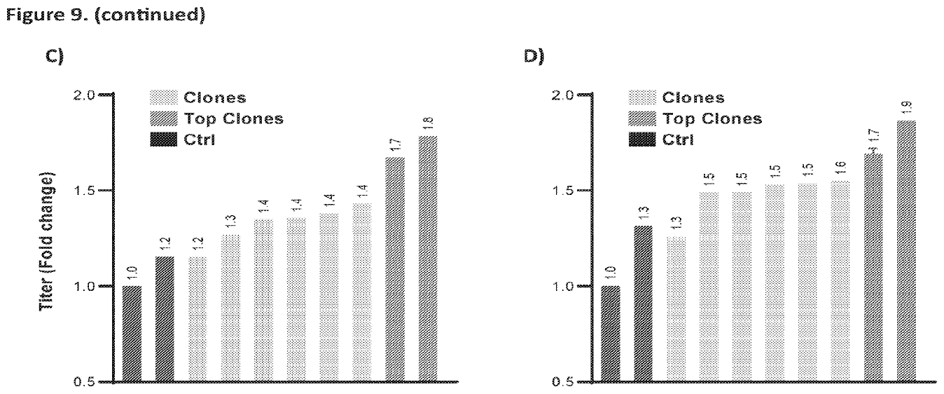

FIG. 9 shows the AAV titer produced by the 4A2-3D3, 4A2-3B11, 4A2-3E6, 4A2-1H7, 4A2-4F12, 4A2-2C12, 4A2-3C3, and 4A2-2H7 DYNLL1+/−/MAX KO clones following transfection with (i) the pHRC #7 (helper+AAV8-trans) plasmid and TG-A cis plasmid. (ii) pHRC #8 (helper+AAV8-trans) plasmid and TG-C cis plasmid, (iii) the helper #5, TG-D cis and AAV9 trans plasmids, and (iv) pHRC #8 (helper+AAV8-trans) plasmid and TG-B cis plasmid. The titer increased similarly for all four transgenes tested in the 4A2-3D3, 4A2-3B11, 4A2-3E6, 4A2-1H7, 4A2-4F12, 4A2-2C12, 4A2-3C3, and 4A2-2H7 clones. The pHRC #7 and pHRD #8 helper/rep/cap plasmids are disclosed in PCT/US2024/023368, filed Apr. 5, 2024.

### Example 5. Host Cell Engineering Through Knocking-Out ANGPT1, ARHGEF7, ARHGEF17, BID, BNIP1, C190RF2, CHST11, CUL1, DAP3, DYNLL1, FIS1, HMGB2, IGFBP3, NRG1, TNFRSF10C, TNFSF12, MAL, MAX, AND/OR VEGFB

Genes with the most pronounced effect on rAAV titers were selected for CRISPR/Cas9 mediated knockout editing. Briefly, on day 1, HEK293 cells will be seeded in collagen-I pre-coated 24-well plates at ~40~50% confluency and reverse co-transfected with Cas9 protein and specific sgRNA targeting the candidate gene. Approximately 48 hrs later, two wells of cells will be harvested for evaluating editing efficiency by PCR and Western blot. On day 4, the rest of cells will be trypsinized and resuspended for single cell cloning into 9-well plates. On about day 14 to 18, the clones will be transferred into 24-well plates. After Sanger sequencing and protein level detection, multiple positive clones specifically knocking-out candidate genes will be picked and transferred into 24-deep well plates. After scale-up into shake flask, the cells will be transfected with a combination of three plasmids (i.e., helper, cis and trans plasmids) to produce AAV8 TG-A, AAV8 TG-B or AAV9 TG-D rAAV particles. 72 hours post triple transfection, cell suspension of each clone will be lysed into AAV-MAX™ lysis buffer and titer of AAV particles produced will be determined using ddPCR.

While the disclosed methods have been described in connection with what is presently considered to be the most practical and preferred embodiments, it is to be understood that the methods encompassed by the disclosure are not to be limited to the disclosed embodiments, but on the contrary, is intended to cover various modifications and equivalent arrangements included within the spirit and scope of the appended claims.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

What is claimed is:

1. A recombinant cell capable of producing rAAV, wherein the cell comprises at least one modification that reduces or eliminates the activity of at least one endogenous gene or gene product in the apoptosis signaling pathway.

2. The cell of claim 1, wherein the cell comprises at least two modifications that reduce or eliminate the activity of at least two genes or gene products in the apoptosis signaling pathway.

3. A cell bank comprising a plurality of cells, wherein the cells comprise at least one modification that reduces or eliminates the activity of at least one endogenous gene or gene product in the apoptosis signaling pathway.

4. The cell bank of claim 3, wherein the cell comprises at least two modifications that reduce or eliminate the activity of at least two genes or gene products in the apoptosis signaling pathway.

5. A cell culture comprising a plurality of cells capable of producing rAAV, wherein the cells comprise at least one modification that reduces or eliminates the activity of at least one endogenous gene or gene product in the apoptosis signaling pathway.

6. The cell culture of claim 5, wherein the cells comprise at least two modifications that reduce or eliminate the activity of at least two genes or gene products in the apoptosis signaling pathway.

7. A method of producing rAAV particles, comprising
a) providing a cell culture comprising a plurality of cells capable of producing rAAV; and
b) maintaining the cell culture under conditions that allow production of the rAAV particles,
wherein the cells comprise at least one modification that reduces or eliminates the activity of at least one endogenous gene or gene product in the apoptosis signaling pathway.

8. A method for producing rAAV particles, comprising culturing a cell capable of producing rAAV particles under conditions that allow the production of the rAAV particles, wherein the cell comprises at least one modification that reduces or eliminates the activity of at least one endogenous gene or gene product in the apoptosis signaling pathway.

9. A method of increasing the production of rAAV particles, comprising
a) providing a cell culture comprising a plurality of cells capable of producing rAAV; and
b) maintaining the cell culture under conditions that allow production of the rAAV particles,
wherein the cell comprises at least one modification that reduces or eliminates the activity of at least one endogenous gene or gene product in the apoptosis signaling pathway.

10. A method of increasing the production of rAAV particles, comprising culturing a cell capable of producing rAAV particles under conditions that allow the production of the rAAV particles, wherein the cell comprises at least one modification that reduces or eliminates the activity of at least one endogenous gene or gene product in the apoptosis signaling pathway.

11. The method of any one of claims 7 to 10, wherein the cell or cells comprise at least two modifications that reduce or eliminate the activity of at least two genes or gene products in the apoptosis signaling pathway.

12. The method of any one of claims 1 to 11, wherein the cell or cells capable of producing rAAV have been transfected with one or more polynucleotides encoding at least one of
a) an rAAV genome to be packaged,
b) adenovirus helper functions necessary for packaging,
c) an AAV rep protein sufficient for packaging, and
d) an AAV cap protein sufficient for packaging.

13. The method of any one of claims 1 to 12, wherein the cell or cells capable of producing rAAV have been transfected with one or more polynucleotides encoding
a) an rAAV genome to be packaged,
b) adenovirus helper functions necessary for packaging,
c) an AAV rep protein sufficient for packaging, and
d) an AAV cap protein sufficient for packaging.

14. A method of producing rAAV particles, comprising
a) providing a cell culture comprising a plurality of cells;
b) introducing into the cells one or more polynucleotides encoding at least one of
i. an rAAV genome to be packaged,
ii. adenovirus helper functions necessary for packaging,
iii. an AAV rep protein sufficient for packaging, and
iv. an AAV cap protein sufficient for packaging; and
c) maintaining the cell culture under conditions that allow production of the rAAV particles,
wherein the cells comprise at least one modification that reduces or eliminates the activity of at least one endogenous gene or gene product in the apoptosis signaling pathway.

15. A method of increasing the production of rAAV particles, comprising
a) providing a cell culture comprising a plurality of cells;
b) introducing into the cells one or more polynucleotides encoding at least one of
i. an rAAV genome to be packaged,
ii. adenovirus helper functions necessary for packaging,
iii. an AAV rep protein sufficient for packaging, and
iv. an AAV cap protein sufficient for packaging; and
c) maintaining the cell culture under conditions that allow production of the rAAV particles
wherein the cells comprise at least one modification that reduces or eliminates the activity of at least one endogenous gene or gene product in the apoptosis signaling pathway.

16. The method of claim 14 or claim 15, wherein the cells comprise at least two modifications that reduce or eliminate the activity of at least two genes or gene products in the apoptosis signaling pathway.

17. The method of any one of claims 14 to 16, comprising introducing into the cells one or more polynucleotides encoding
i. an rAAV genome to be packaged,
ii. adenovirus helper functions necessary for packaging,
iii. an AAV rep protein sufficient for packaging, and
iv. an AAV cap protein sufficient for packaging.

18. The method of any one of claims 14 to 17, wherein the introducing one or more polynucleotides into the cells is by transfection.

19. The cell, cell bank, cell culture or method of any one of claims 1 to 18, wherein the modification comprises a mutation in the gene.

20. The cell, cell bank, cell culture or method of claim 19, wherein the genetic modification comprises a missense mutation, nonsense mutation, or frameshift mutation.

21. The cell, cell bank, cell culture or method of claim 19, wherein the genetic modification comprises a deletion.

22. The cell, cell bank, cell culture or method of any one of claims 19 to 21, wherein the mutation is a heterozygous mutation.

23. The cell, cell bank, cell culture or method of claim 22, wherein the heterozygous mutation affects one copy of the gene.

24. The cell, cell bank, cell culture or method of claim 22, wherein the heterozygous mutation affects more than on copy of the gene.

25. The cell, cell bank, cell culture or method of claim 22, wherein the heterozygous mutation affects 1, 2 or 3 copies of the gene.

26. The cell, cell bank, cell culture or method of any one of claims 19 to 21, wherein the mutation is a homozygous mutation.

27. The cell, cell bank, cell culture or method of any one of claims 1 to 22, wherein the modification comprises an inhibitory nucleic acid molecule capable of reducing or eliminating the activity of the gene or gene product.

28. The cell, cell bank, cell culture or method of claim 27, wherein the modification comprises an anti-sense RNA.

29. The cell, cell bank, cell culture or method of claim 27, wherein the modification comprises a small interfering RNA (siRNA), microRNA (miRNA), or short hairpin RNA (shRNA).

30. The cell, cell bank, cell culture or method of any one of claims 1 to 29, wherein the at least one endogenous gene or gene product in the apoptosis signaling pathway is selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product.

31. The cell, cell bank, cell culture or method of claim 30, wherein the at least one endogenous gene or gene product comprises any two genes or gene products selected from the group consisting of the ANGPT1, ARHGEF7, ARHGEF17, BID, BNIP1, C19orf2, CHST11, CUL1, DAP3, DYNLL1, FIS1, HMGB2, IGFBP3, NRG1, TNFRSF10C, TNFSF12, MAL, MAX, and VEGFB gene or gene product.

32. The cell, cell bank, cell culture or method of any one of claims 1 to 29, wherein the at least one endogenous gene or gene product is selected from the group consisting of the ARHGEF7, ARHGEF17, BNIP1, C19orf2, CHST11, DYNLL1, IGFBP3, MAX, and VEGFB gene or gene product.

33. The cell, cell bank, cell culture or method of claim 32, wherein the at least one endogenous gene or gene product comprises any two genes or gene products selected from the group consisting of the ARHGEF7, ARHGEF17, BNIP1, C19orf2, CHST11, DYNLL1, IGFBP3, MAX, and VEGFB gene or gene product.

34. The cell, cell bank, cell culture or method of any one of claims 1 to 29, wherein the at least one endogenous gene or gene product comprises DYNLL1, IGFBP3, and/or MAX gene or gene product.

35. The cell, cell bank, cell culture or method of claim 34, wherein the at least one endogenous gene or gene product comprises DYNLL1 gene or gene product.

36. The cell, cell bank, cell culture or method of claim 35, wherein the modification comprises a heterozygous DYNLL1 mutation.

37. The cell, cell bank, cell culture or method of claim 34, wherein the at least one endogenous gene or gene product comprises DYNLL1 and MAX gene or gene product.

38. The cell, cell bank, cell culture or method of claim 37, wherein the modification comprises a heterozygous DYNLL1 mutation and a MAX mutation.

39. The cell, cell bank, cell culture or method of any one of claims 1 to 29, wherein the at least one endogenous gene or gene product comprises DYNLL1 and a second gene or gene product selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB.

40. The cell, cell bank, cell culture or method of any one of claims 1 to 29, wherein the at least one endogenous gene or gene product comprises DYNLL1 and a second gene or gene product selected from the group consisting of ARHGEF7, ARHGEF17, BNIP1, C19orf2, CHST11, IGFBP3, MAX, and VEGFB.

41. The cell, cell bank, cell culture or method of any one of claims 1 to 29, wherein the at least one endogenous gene or gene product comprises DYNLL1 and ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB.

42. The cell, cell bank, cell culture or method of any one of claims 1 to 29, wherein the at least one endogenous gene or gene product comprises DYNLL1 and one or more genes or gene products selected from the group consisting of ANGPT1, ARHGEF7, ARHGEF17, BID, BNIP1, C19orf2, CHST11, CUL1, DAP3, FIS1, HMGB2, IGFBP3, NRG1, TNFRSF10C, TNFSF12, MAL, MAX, and VEGFB.

43. The cell, cell bank, cell culture or method of any one of claims 1 to 29, wherein the at least one endogenous gene or gene product comprises DYNLL1 and one or more genes or gene products selected from the group consisting of ARHGEF7, ARHGEF17, BNIP1, C19orf2, CHST11, IGFBP3, MAX, and VEGFB.

44. The cell, cell bank, cell culture or method of any one of claims 1 to 29, wherein the at least one endogenous gene or gene product comprises DYNLL1 and ARHGEF7.

45. The cell, cell bank, cell culture or method of any one of claims 1 to 29, wherein the at least one endogenous gene or gene product comprises DYNLL1 and ARHGEF17.

46. The cell, cell bank, cell culture or method of any one of claims 1 to 29, wherein the at least one endogenous gene or gene product comprises DYNLL1 and BNIP1.

47. The cell, cell bank, cell culture or method of any one of claims 1 to 29, wherein the at least one endogenous gene or gene product comprises DYNLL1 and C19orf2.

48. The cell, cell bank, cell culture or method of any one of claims 1 to 29, wherein the at least one endogenous gene or gene product comprises DYNLL1 and CHST11.

49. The cell, cell bank, cell culture or method of any one of claims 1 to 29, wherein the at least one endogenous gene or gene product comprises DYNLL1 and IGFBP3.

50. The cell, cell bank, cell culture or method of any one of claims 1 to 29, wherein the at least one endogenous gene or gene product comprises DYNLL1 and MAX.

51. The cell, cell bank, cell culture or method of any one of claims 1 to 29, wherein the at least one endogenous gene or gene product comprises DYNLL1 and VEGFB.

52. The cell, cell bank, cell culture or method of any one of claims 1 to 51, wherein the cell is a mammalian cell.

53. The cell, cell bank, cell culture or method of any one of claims 1 to 51, wherein the cell is an insect cell.

54. The cell, cell bank, cell culture or method of any one of claims 1 to 51, wherein the cell is a HEK293 cell, HEK293 derived cell, CHO cell, CHO derived cell, HeLa cell, SF-9 cell, BHK cell, Vero cell, CAP cell or PerC6 cell.

55. The cell, cell bank, cell culture or method of any one of claims 1 to 51, wherein the cell is a HEK293 cell.

56. The cell, cell bank, cell culture or method of any one of claims 1 to 55, wherein the cell culture is a suspension culture.

57. The cell, cell bank, cell culture or method of any one of claims 12 to 51, wherein the adenovirus helper functions comprise at least one of an adenovirus E4 gene, E2a gene, and VA gene.

58. The cell, cell bank, cell culture or method of any one of claims 12 to 51, wherein the adenovirus helper functions comprise an adenovirus E4 gene, E2a gene, and VA gene.

59. The cell, cell bank, cell culture or method of claim 58, wherein the polynucleotide encoding the adenovirus helper functions comprises pAD Delta F6 or Helper #5.

60. The cell, cell bank, cell culture or method of claim 58, wherein the polynucleotide encoding the adenovirus helper functions comprises pHRC #7 or pHRC #8.

61. The method of any one of claims 7 to 60, wherein the maintaining the cell culture or culturing the cell under conditions that allow production of the rAAV particles is for between about 2 days and about 10 days, between about 2 days and about 15 days, or between about 5 days and 14 days.

62. The method of any one of claims 7 to 60, wherein the maintaining the cell culture or culturing the cell under conditions that allow production of the rAAV particles is for about 2 days, about 3 days, about 4 days, about 5 days, about 6 days, or about 7 days.

63. The method of any one of claims 7 to 60, wherein the maintaining the cell culture or culturing the cell under conditions that allow production of the rAAV particles is for about 2 days, about 3 days, about 4 days, about 5 days, about 6 days, or about 7 days.

64. The method of claim 63, wherein the maintaining the cell culture or culturing the cell under conditions that allow production of the rAAV particles is for about 5 days.

65. The method of any one of claims 7 to 64, further comprising recovering the rAAV particles.

66. The method of any one of claims 7 to 65, wherein the method produces more rAAV particles measured as GC/ml than a reference method using a cell that does not comprise the modification.

67. The method of any one of claims 7 to 65, wherein the cell culture produces at least 10%, at least 20%, at least 30%, at least 50%, at least 75%, or at least 100% more rAAV particles measured as GC/ml than a reference method using a cell that does not comprise the modification.

68. The cell culture or method of any one of claims 5 to 67, wherein the cell culture has a volume between about 50 liters and about 20,000 liters.

69. The cell culture or method of any one of claims 5 to 67, wherein the cell culture has a volume between about 200 liters and about 2,000 liters.

70. The cell, cell bank, cell culture or method of any one of claims 1 to 69, wherein the rAAV comprises a capsid protein of the AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu32, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 serotype.

71. The cell, cell bank, cell culture or method of any one of claims 1 to 69, wherein the rAAV comprises a capsid protein of the AAV8, AAV9, AAV.rh10, AAV.rh20, AAV.rh39, AAV.Rh74, AAV.RHM4-1, AAV.hu32, and AAV.hu37 serotype.

72. The cell, cell bank, cell culture or method of any one of claims 1 to 69, wherein the rAAV s comprises a capsid protein of the AAV8 or AAV9 serotype.

73. The cell, cell bank, cell culture or method of any one of claims 1 to 72, wherein the rAAV comprises a genome encoding a polypeptide or a double stranded RNA molecule.

74. The cell, cell bank, cell culture or method of claim 73, wherein the genome encodes a polypeptide.

75. The cell, cell bank, cell culture or method of claim 73, wherein the genome encodes an anti-VEGF Fab, anti-kallikrein antibody, anti-TNF antibody, microdystrophin, minidystrophin, iduronidase (IDUA), iduronate 2-sulfatase (IDS), low-density lipoprotein receptor (LDLR), tripeptidyl peptidase 1 (TPP1), or non-membrane associated splice variant of VEGF receptor 1 (sFlt-1).

76. The cell, cell bank, cell culture or method of claim 73, wherein the genome encodes an gamma-sarcoglycan, Rab Escort Protein 1 (REP1/CH-M), retinoid isomerohydrolase (RPE65), cyclic nucleotide gated channel alpha 3 (CNGA3), cyclic nucleotide gated channel beta 3 (CNGB3), aromatic L-amino acid decarboxylase (AADC), lysosome-associated membrane protein 2 isoform B (LAMP2B), Factor VIII, Factor IX, retinitis pigmentosa GTPase regulator (RPGR), retinoschisin (RS1), sarcoplasmic reticulum calcium ATPase (SERCA2a), aflibercept, battenin (CLN3), transmembrane ER protein (CLN6), glutamic acid decarboxylase (GAD), Glial cell line-derived neurotrophic factor (GDNF), aquaporin 1 (AQP1), dystrophin, myotubularin 1 (MTM1), follistatin (FST), glucose-6-phosphatase (G6Pase), apolipoprotein A2 (APOA2), uridine diphosphate glucuronosyl transferase 1A1 (UGT1A1), arylsulfatase B (ARSB), N-acetyl-alpha-glucosaminidase (NAGLU), alpha-glucosidase (GAA), alpha-galactosidase (GLA), beta-galactosidase (GLB1), lipoprotein lipase (LPL), alpha 1-antitrypsin (AAT), phosphodiesterase 6B (PDE6B), omithine carbamoyltransferase 90TC), survival motor neuron (SMN1), survival motor neuron (SMN2), neurturin (NRTN), Neurotrophin-3 (NT-3/NTF3), porphobilinogen deaminase (PBGD), nerve growth factor (NGF), mitochondrially encoded NADH:ubiquinone oxidoreductase core subunit 4 (MT-ND4), protective protein cathepsin A (PPCA), dysferlin, MER proto-oncogene, tyrosine kinase (MERTK), cystic fibrosis transmembrane conductance regulator (CFTR), or tumor necrosis factor receptor (TNFR)-immunoglobulin (IgG1) Fc fusion.

77. The cell, cell bank, cell culture or method of claim 73, wherein the genome encodes a dystrophin or a microdystrophin.

78. The cell, cell bank, cell culture or method of claim 73, wherein the genome to be packaged encodes a microRNA.
